(19) United States
(12) Patent Application Publication
(10) Pub. No.: US 2025/0000798 A1
(43) Pub. Date: Jan. 2, 2025

(54) METHODS RELATED TO MEGAKARYOCYTE-DERIVED EXTRACELLULAR VESICLES

(71) Applicant: STRM.bio Incorporated, Cambridge, MA (US)

(72) Inventors: Jonathan THON, Cambridge, MA (US); Daniel BODE, York (GB); Iain THOMPSON, Cambridge, MA (US); Laura GOLDBERG, Cambridge, MA (US)

(21) Appl. No.: 18/708,866

(22) PCT Filed: Nov. 11, 2022
(86) PCT No.: PCT/US22/49729

§ 371 Date: May 9, 2024

Related U.S. Application Data
(60) Provisional application No. 63/380,577, filed on Oct. 23, 2022.
(60) Provisional application No. 63/278,364, filed on Nov. 11, 2021.

(51) Int. Cl. A61K 9/127; A61K 9/51; A61K 31/7105; A61K 45/06; B82Y 5/00; C12N 5/078; C12N 9/22; C12N 15/11

(52) U.S. Cl. CPC A61K 9/1272; A61K 9/5176; A61K 9/5192; A61K 31/7105; A61K 45/06; C12N 5/0644; C12N 9/22; C12N 15/111; B82Y 5/00; C12N 2310/20; C12N 2501/599; C12N 2506/02; C12N 2506/1369; C12N 2506/45

(57) ABSTRACT

Disclosed herein are methods related to megakaryocyte-derived extracellular vesicles derived from human pluripotent stem cells. In some aspects the disclosed methods relate to the generation, the purification and cardo loading of the megakaryocyte-derived extracellular vesicles.

### CROSS-REFERENCE TO RELATED APPLICATIONS

This application claims the benefit of priority to U.S. Provisional Patent Application Nos. 63/278,364, filed Nov. 11, 2021, and 63/380,577, filed Oct. 23, 2022, all of which are incorporated by reference herein in their entireties.

### FIELD

The present disclosure relates to methods of generating megakaryocyte-derived extracellular vesicles derived from human pluripotent stem cells.

### BACKGROUND

In order to avoid drug cytotoxicity and side effects and/or rapid clearance, a variety of nano-vesicles have been developed to administer therapeutic agents. These present several advantages, including reducing renal clearance, improving site-specific delivery, simultaneous delivery of multiple therapeutic agents, protection from enzymatic degradation, immune-evasion, sequential multistage release, stimuli-responsive activation, and theranostic capabilities, among others

However, clinical use of these nano-vesicles has been limited partially due to complex and costly manufacturing required to achieve multi-functionality. The largest category of clinically approved nano-vesicles is liposomes, which consist of a simple lipid bilayer surrounding an aqueous compartment. Liposomes are versatile drug delivery vehicles, as both the lipid membrane and interior space can be utilized for loading of hydrophobic and hydrophilic drugs, respectively. However, liposomes can also trigger adverse effects in a patient, including immune reactions and cytotoxicity, in addition to target non-specificity and inefficient unloading of therapeutic agents, because liposomes are foreign, synthetic entities, with limited cell or tissue targeting machinery. Adenovirus, retrovirus, AAV, and lentivirus vectors are currently the most popular viral vectors for gene therapy. Nevertheless, conventional methods of viral vector production using adherent cell lines and transient transfections in the presence of serum are not scalable.

Additionally, to achieve improved therapy, it important to safely and effectively package a nucleic acid and/or a protein cargo with the nanovesicles and release it in a controlled and timely manner.

Accordingly, there is a need for methods that enhance the effectiveness of cargo packaging and delivery to cells. There is also an urgent need for delivery vehicles that can be generated cost-effectively at scale and that eliminate or reduce adverse effects when administered to a patient.

### SUMMARY

Disclosed herein are methods of generating megakaryocyte-derived extracellular vesicles derived from human pluripotent stem cells. Also disclosed herein are methods of purifying and loading megakaryocyte-derived extracellular vesicles derived from human pluripotent stem cells.

In one aspect, the present invention relates to methods of generating a plurality of megakaryocyte-derived extracellular vesicles by (a) differentiating human pluripotent stem cells to megakaryocytes; and (b) isolating megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation.

In another aspect, the present invention relates to methods of generating a plurality of megakaryocyte-derived extracellular vesicles. The methods comprise (a) obtaining human pluripotent stem cells, the human pluripotent stem cells being primary CD34+ hematopoietic stem cells; (b) differentiating the human pluripotent stem cells to megakaryocytes; and (c) isolating megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation. In embodiments, the primary CD34+ hematopoietic stem cells are sourced from peripheral blood or cord blood. In embodiments, the human pluripotent stem cells are selected from induced pluripotent stem cells (iPSCs), hematopoietic stem cells (HSCs), and embryonic stem cells (ESCs).

In another aspect, the present invention relates to further methods of generating a plurality of megakaryocyte-derived extracellular vesicles. The methods comprise (a) differentiating human pluripotent stem cells to megakaryocytes in culture; (b) enriching the culture for megakaryocytes; (c) isolating megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation.

In yet another aspect, the present invention relates to methods for purifying a plurality of megakaryocyte-derived extracellular vesicles. The methods comprise (a) obtaining a material comprising a population of megakaryocytes in culture or the cell culture media thereof; (b) filtering the material of step (a) thereby yielding predominantly CD41+ megakaryocyte-derived extracellular vesicles in the filtrate.

In another aspect, the present invention relates to methods of loading cargo in a plurality of megakaryocyte-derived extracellular vesicles. The methods comprise (a) obtaining a plurality of megakaryocyte-derived extracellular vesicles; (b) contacting the plurality of megakaryocyte-derived extracellular vesicles with a cargo of interest; and (c) applying an electrical pulse for a period of time, thereby permitting the cargo to pass into the lumen of the megakaryocyte-derived extracellular vesicles, wherein the electrical pulse is applied for about 5 to about 25 milliseconds and the electrical pulse is applied about 5 to about 25 times

In a further aspect, the present invention relates to methods of loading cargo in a plurality of megakaryocyte-derived extracellular vesicles. The methods comprise (a) obtaining a plurality of megakaryocyte-derived extracellular vesicles; (b) contacting the plurality of megakaryocyte-derived extracellular vesicles with a cargo of interest; and (c) applying an electrical pulse for a period of time, thereby permitting the cargo to pass into the lumen of the megakaryocyte-derived extracellular vesicles, wherein the electrical pulse is applied for at least about 15 milliseconds and the electrical pulse is applied for at least about 10 times.

### BRIEF DESCRIPTION OF THE DRAWINGS

The summary, as well as the following detailed description, are further understood when read in conjunction with the appended drawings. For the purpose of illustrating the disclosed methods, there are shown in the drawings' exemplary embodiments of the methods however, these should not be limited to the specific embodiments disclosed.

In the drawings:

FIGS. 1A-1C are series of graphs showing that there was inter-batch consistency in yield and surface marker expression at Day 17-Day 18 harvest. The concentration of Megakaryocyte-Derived Extracellular Vesicles (MkEVs) MkEVs/mL ranged between 2×10⁸ to 4×10⁸ MkEVs/mL and the total MkEV yield was approximately 5×10¹⁰ to 8×10¹⁰ MkEVs (FIG. 1A) Bars represent average MkEVs/mL (left) and total MkEVs (right)±SD from 5 independent manufacturing runs. In addition, the percent of CD41+ MkEVs expressing individual surface markers were consistent across 5 independent MkEV batches. Lines represent mean±SD from 5 independent manufacturing runs (FIG. 1B). Finally, the MkEV product was DRAQ5 negative by flow cytometry, indicating lack of cellular DNA contamination (FIG. 1C).

FIGS. 2A-2B are series of graphs showing that the yield of CD41+ MkEVs continued to rise with increasing days of megakaryocyte differentiation culture. The MkEV yield continued to rise between 11 and 18 days in differentiation culture (FIG. 2A) without a decrease in the purity of CD41+ events (FIG. 2B). The average yield±SD from two independent manufacturing runs is shown.

FIGS. 3A-3B are series of graphs demonstrating that the viability of cells in culture began to decline after differentiation day 12 (FIG. 3A) and the viability of the CD41+ cell population in culture specifically began to decline between differentiation day 8-13 (FIG. 3B), indicating that the MkEV yield was not linearly correlated with cell viability. Data from 2-5 independent manufacturing runs are shown.

FIGS. 4A-4B are series of histograms showing that a low shear pump for tangential flow filtration improved MkEV purity without sacrificing CD41+ quantity. MkEV samples were processed using a peristaltic pump or magnetic pump and the number of total events/mL (FIG. 4A) and the number of CD41+ events/mL (FIG. 4B) were quantified by flow cytometry (Amnis® ImageStream Imaging Flow Cytometer).

FIGS. 5A-5E are series of a diagram and histograms depicting the tuneable loading of plasmid DNA (pDNA) into MkEVs by electroporation. As shown in FIG. 5A, pDNA (5.5 kb) was electroporated into MkEVs. Following electroporation, samples were treated with DNase to remove any free pDNA or MkEV surface-associated pDNA. Following DNAse treatment, DNA was extracted and pDNA amount was quantified by qPCR. The number of pDNA copies were calculated based on a standard curve run in parallel and divided by the total number of MkEVs in the sample to give pDNA copy number/MkEV. MkEVs plus pDNA without electroporation served as a control. As shown increasing the pulse number (FIGS. 5B and 5C) and increasing the pDNA cargo:MkEV ratio (FIG. D) both led to increased pDNA loading. Pulse length of 5 ms provided the highest loading of pDNA into MkEVs while pulse length of 15 ms led to a lower number of pDNA copies loaded into MkEVs (FIG. 5E). Average pDNA copy number per MkEV±standard deviation is shown.

FIGS. 6A-6B are series of histograms depicting the loading of a 9.8 kb (FIG. 6A) and an 8.3 kb (FIG. 6B) pDNA. FIG. 6A shows a 9.8 kb pDNA electroporated (EP) into MkEVs using 200V and either 0 pulses (no electroporation control), 4 pulses, or 10 pulses. All samples were treated with DNase to remove any noninternalized pDNA cargo prior to DNA extraction. DNA was then extracted and qPCR was performed. Loaded pDNA was quantified (ng) based on a standard curve run in parallel, and number of pDNA copies/MkEV was calculated. Tuneable loading was shown, with increasing pDNA copies/MkEV loaded with increasing pulse number. FIG. 6B shows an 8.3 kB pDNA electroporated (EP) into MkEVs using either 200V or 400V. Controls included MkEVs+ pDNA without electroporation, MkEVs alone, pDNA alone. All samples were treated with DNase to remove any noninternalized pDNA cargo prior to DNA extraction. DNA was then extracted, and qPCR was performed. Loaded pDNA was quantified (ng) based on a standard curve run in parallel, and number of pDNA copies/MkEV was calculated. Protected pDNA was recovered from the MkEV+ pDNA+ electroporation sample indicating successful internal loading with these parameters.

FIGS. 7A-7F are series of western blot images demonstrating successful loading of Cas9 into MkEVs. MkEVs were loaded with Cas9 by electroporation at 750V (FIGS. 7A-7C) or 1000 V (FIGS. 7D-7F), and subjected to proteinase K treatment at 1 or 5 ug/mL to digest any unbound or externally associated Cas9. Control samples included MkEVs alone±Proteinase K, Cas9 alone±Proteinase K, and MkEVs+Cas9 without electroporation±Proteinase K. The MkEVs were then analyzed for internalized, protease-protected Cas9 by western blotting (FIGS. 7A and 7D). Actin was quantified as a non-loaded control (FIGS. 7B and 7E). The absence of Cas9 in un-electroporated but its presence in electroporated samples following protease digestion indicates successful loading of Cas9 inside the MkEV by electroporation. Ponceau staining showed similar loading across all wells (FIGS. 7C and 7F).

FIGS. 8A-8C show that electroporation does not alter EV targeting to HSPCs following in vivo intravenous administration. A non-limiting schematic of the methods are shown in FIG. 8A. MkEVs were labeled with DiD (1,1-Dioctadecyl-3,3,3,3-tetramethylindodicarbocyanine) and either unelectroporated or electroporated (EP). They were injected into wild type mice via tail vein injection and tissues were collected 16 hrs post injection and analyzed for EV uptake (DiD+) by flow cytometry. Injection of DiD processed in parallel without MkEVs served as a negative control. Biodistribution in bone marrow (FIG. 8B) and biodistribution in multiple tissues (FIG. 8C) are shown. Bars represent percent of cells analyzed per tissue that were DiD+ cells, as quantified by flow cytometry in mice injected with either dye alone processed without MkEVs (white bars), DiD-labeled, unelectroporated EVs (gray bars) or DiD-labeled electroporated EVs (black bars). n=1 mouse/group.

### DETAILED DESCRIPTION

The present invention is based, in part on the discovery of methods for generating, and/or purifying megakaryocyte-derived extracellular vesicles that are isolated by specific means and/or at specific time after commencement of the differentiation and that are characterized by particular sets of physical characteristics, such as percentage of viability, some biomarker composition (e.g. the presence, absence, or amount of a biomarker) and size. In some aspects, the present invention provides methods of effectively loading cargo in megakaryocyte-derived extracellular vesicles under specific settings such as an electrical pulse, thereby favoring optimal therapeutic applications.

### Methods of Generating Megakaryocyte-Derived Extracellular Vesicles

As described in details elsewhere herein, in embodiments megakaryocytes are derived from pluripotent hematopoietic stem cell (HSC) precursors. In embodiments, various molecular signals, such as cytokines or Thrombopoietin (TPO), induce HSCs to differentiate into megakaryocytes.

In embodiments, the disclosed megakaryocyte-derived extracellular vesicles are generated from mature megakaryocytes. In embodiments, the megakaryocyte-derived extracellular vesicles are generated from immature megakaryocytes.

In embodiments, the megakaryocyte-derived extracellular vesicles are generated by: (a) obtaining a human pluripotent stem cell being a primary CD34+ HSC sourced from peripheral blood or cord blood; (b) differentiating the human pluripotent stem cell to a megakaryocyte in the absence of added EPO and in the presence of added TPO; and (c) isolating the megakaryocyte-derived extracellular vesicles from the megakaryocytes.

In embodiments, the method is an in vivo method. In embodiments, the method is an ex vivo method.

In embodiments, the CD34+ HSC sourced from peripheral blood are multipotent stem cells derived from volunteers whose stem cells are mobilized into the bloodstream by administration of a mobilization agent such as granulocyte colony stimulating factor (G-CSF) and granulocyte-macrophage colony stimulating factor (GM-CSF).

In embodiments, the cord blood comprises multipotent stem cells derived from blood that remains in the placenta and the attached umbilical cord after childbirth.

In embodiments, the megakaryocyte-derived extracellular vesicles are autologous with the patient. In embodiments, human pluripotent stem cells are extracted from the patient and used to generate megakaryocytes, from which megakaryocyte-derived extracellular vesicles comprising a cargo of choice are generated and then administered to the patient. In embodiments, differentiated cells are extracted from the patient and used to generate iPSCs, which in turn are used to generate megakaryocytes, from which megakaryocyte-derived extracellular vesicles comprising a cargo of choice are generated and then administered to the patient.

In embodiments, the megakaryocyte-derived extracellular vesicles are allogeneic with the patient. In embodiments, human pluripotent stem cells are extracted from a human subject who is not the patient and used to generate megakaryocytes, from which megakaryocyte-derived extracellular vesicles comprising a cargo of choice are generated and then administered to the patient. In embodiments, differentiated cells are extracted from a human subject who is not the patient and used to generate iPSCs, which in turn are used to generate megakaryocytes, from which megakaryocyte-derived extracellular vesicles comprising a cargo of choice are generated and then administered to the patient.

In embodiments, the megakaryocyte-derived extracellular vesicles are heterologous with the patient. In embodiments, pluripotent stem cells are extracted from a non-human subject and used to generate megakaryocytes, from which megakaryocyte-derived extracellular vesicles comprising a cargo of choice are generated and then administered to the patient. In embodiments, differentiated cells are extracted from a non-human subject and used to generate iPSCs, which in turn are used to generate megakaryocytes, from which megakaryocyte-derived extracellular vesicles comprising a cargo of choice are generated and then administered to the patient.

In embodiments, the incubating comprises one or more of sonication, saponin permeabilization, mechanical vibration, hypotonic dialysis, extrusion through porous membranes, cholesterol conjugation, application of electric current and combinations thereof. In embodiments, the incubating comprises one or more of electroporating, transforming, transfecting, and microinjecting.

In embodiments, the method further comprises (d) contacting the megakaryocyte-derived extracellular vesicles with radiation. In embodiments, the radiation is gamma radiation. In embodiments, the gamma radiation is at an amount greater than 12 kGy, 25 kGy, or 50 kGy. In embodiments, the gamma radiation is at an amount between about 12 kGy and 15 kGy. In embodiments, the gamma radiation is at an amount between about 15 kGy and 20 kGy. In embodiments, the gamma radiation is at an amount between about 20 kGy and 25 kGy. In embodiments, the gamma radiation is at an amount between about 25 kGy and 30 kGy. In embodiments, the gamma radiation is at an amount between about 30 kGy and 35 kGy. In embodiments, the gamma radiation is at an amount between about 35 kGy and 40 kGy. In embodiments, the gamma radiation is at an amount between about 40 kGy and 45 kGy. In embodiments, the gamma radiation is at an amount between about 45 kGy and 50 kGy. In embodiments, the gamma radiation is at an amount between about 50 kGy and 55 kGy. In embodiments, the gamma radiation is at an amount between about 55 kGy and 60 kGy.

In embodiments, the disclosed methods to generate megakaryocyte-derived extracellular vesicles are standardized to enable large-scale production.

In one aspect, the present invention relates to methods of generating a plurality of megakaryocyte-derived extracellular vesicles. In embodiments, the methods comprise (a) megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation.

In another aspect, methods of generating a plurality of megakaryocyte-derived extracellular vesicles are provided. The methods comprise (a) obtaining human pluripotent stem cells, the human pluripotent stem cells being primary CD34+ hematopoietic stem cells; (b) differentiating the human pluripotent stem cells to megakaryocytes; and (c) isolating megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation. In embodiments, the primary CD34+ hematopoietic stem cells are sourced from peripheral blood or cord blood.

In yet another aspect, methods of generating a plurality of megakaryocyte-derived extracellular vesicles are provided. The methods comprise (a) differentiating human pluripotent stem cells to megakaryocytes in culture; (b) enriching the culture for megakaryocytes; (c) isolating megakaryocyte-derived extracellular vesicles from the megakaryocytes, wherein the isolation is from megakaryocytes in a culture having greater than about 20% viability or more, and/or the isolation occurs at less than 12 days after commencement of the differentiation or at least about 13 days to about 24 days after commencement of the differentiation.

In embodiments, the megakaryocyte-derived extracellular vesicles are isolated from the source cell, such as a megakaryocyte, using a method which is substantially free of the external application of biomechanical stress (e.g. to the source cell). Non-limiting examples of methods of isolation that are substantially free of the external application of biomechanical stress include tangential flow filtration, differential centrifugation, immunomagnetic cell sorting method or bead-based selection. In embodiments, the enrichment of the cell culture uses a bead-based selection of megakaryocytes. In embodiments the beads are coated with an anti-CD61 agent or an anti-CD41 agent.

In embodiments, the megakaryocyte-derived extracellular vesicles are isolated from the megakaryocytes at a specific time after commencement of the differentiation and at a specific stage of megakaryocytes cell culture viability.

### Viability

In embodiments, the isolation of megakaryocyte-derived extracellular vesicles from the megakaryocytes is performed in a culture having greater than about 15% viability or more. In embodiments, the isolation of megakaryocyte-derived extracellular vesicles from the megakaryocytes is performed in a culture having greater than about 20% viability or more. In embodiments, the isolation of megakaryocyte-derived extracellular vesicles from the megakaryocytes is performed in a culture having greater than about 25% viability or more. In embodiments, the isolation of megakaryocyte-derived extracellular vesicles from the megakaryocytes is performed in a culture having greater than about 30% viability or more. In embodiments, the isolation of megakaryocyte-derived extracellular vesicles from the megakaryocytes is performed in a culture having greater than about 35% viability or more. In embodiments, the isolation of megakaryocyte-derived extracellular vesicles from the megakaryocytes is performed in a culture having greater than about 40% viability or more. In embodiments, the isolation of megakaryocyte-derived extracellular vesicles from the megakaryocytes is performed in a culture having greater than about 45% viability or more. In embodiments, the isolation of megakaryocyte-derived extracellular vesicles from the megakaryocytes is performed in a culture having greater than about 50% viability or more. In embodiments, the isolation of megakaryocyte-derived extracellular vesicles from the megakaryocytes is performed in a culture having greater than about 55% viability or more. In embodiments, the isolation of megakaryocyte-derived extracellular vesicles from the megakaryocytes is performed in a culture having greater than about 60% viability or more. In embodiments, the isolation of megakaryocyte-derived extracellular vesicles from the megakaryocytes is performed in a culture having greater than about 65% viability or more. In embodiments, the isolation of megakaryocyte-derived extracellular vesicles from the megakaryocytes is performed in a culture having greater than about 70% viability or more. In embodiments, the isolation of megakaryocyte-derived extracellular vesicles from the megakaryocytes is performed in a culture having greater than about 75% viability or more. In embodiments, the isolation of megakaryocyte-derived extracellular vesicles from the megakaryocytes is performed in a culture having greater than about 80% viability or more. In embodiments, the isolation of megakaryocyte-derived extracellular vesicles from the megakaryocytes is performed in a culture having greater than about 85% viability or more. In embodiments, the isolation of megakaryocyte-derived extracellular vesicles from the megakaryocytes is performed in a culture having greater than about 90% viability or more. In embodiments, the isolation of megakaryocyte-derived extracellular vesicles from the megakaryocytes is performed in a culture having greater than about 95% viability or more.

In other embodiments, the isolation of megakaryocyte-derived extracellular vesicles from the megakaryocytes is performed in a culture having a viability about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% or more.

In embodiments, the isolation of megakaryocyte-derived extracellular vesicles from the megakaryocytes is performed in a culture having a viability greater than about 15%, greater than about 16%, greater than about 17%, greater than about 18%, greater than about 19%, greater than about 20%, greater than about 21%, greater than about 22%, greater than about 23%, greater than about 24%, greater than about 25%, greater than about 26%, greater than about 27%, greater than about 28%, greater than about 29%, greater than about 30%, greater than about 31%, greater than about 32%, greater than about 33%, greater than about 34%, greater than about 35%, greater than about 36%, greater than about 37%, greater than about 38%, greater than about 39%, greater than about 40%, greater than about 41%, greater than about 42%, greater than about 43%, greater than about 44%, greater than about 45%, greater than about 46%, greater than about 47%, greater than about 48%, greater than about 49%, greater than about 50%, greater than about 51%, greater than about 52%, greater than about 53%, greater than about 54%, greater than about 55%, greater than about 56%, greater than about 57%, greater than about 58%, greater than about 59%, greater than about 60%, greater than about 61%, greater than about 62%, greater than about 63%, greater than about 64%, greater than about 65%, greater than about 66%, greater than about 67%, greater than about 68%, greater than about 69%, greater than about 70%, greater than about 71%, greater than about 72%, greater than about 73%, greater than about 74%, greater than about 75%, greater than about 76%, greater than about 77%, greater than about 78%, greater than about 79%, greater than about 80%, greater than about 81%, greater than about 82%, greater than about 83%, greater than about 84%, greater than about 85%, greater than about 86%, greater than about 87%, greater than about 88%, greater than about 89%, greater than about 90%, greater than about 91%, greater than about 92%, greater than about 93%, greater than about 94%, greater than about 95%, greater than about 96%, greater than about 97%, greater than about 98%, or greater than about 99% or more.

In embodiments, the isolation of megakaryocyte-derived extracellular vesicles from the megakaryocytes is performed in a culture having a viability of about 50% or less, e.g. about 50%, or about 45%, or about 40%, or about 30%. In embodiments, the isolation of megakaryocyte-derived extracellular vesicles from the megakaryocytes is performed in a culture having a viability of about 49% or less, about 48% or less, about 47% or less, about 46% or less, about 45% or less, about 44% or less, about 43% or less, about 42% or less, about 41% or less, about 40% or less, about 39% or less, about 38% or less, about 37% or less, about 36% or less, about 35% or less, about 34% or less, about 33% or less, about 32% or less, about 31% or less, about 30% or less, about 29% or less, about 28% or less, about 27% or less, about 26% or less, about 25% or less, about 24% or less, about 23% or less, about 22% or less, about 21% or less, about 20% or less, about 19% or less, about 18% or less, about 17% or less, about 16% or less, about 15% or less, about 14% or less, about 13% or less, about 12% or less, about 11% or less, or about 10% or less.

In embodiments, the isolation of megakaryocyte-derived extracellular vesicles from the megakaryocytes is performed in a culture having a viability of about 50% or less. In embodiments, the isolation of megakaryocyte-derived extracellular vesicles from the megakaryocytes is performed in a culture having a viability of about 50%. In embodiments, the isolation of megakaryocyte-derived extracellular vesicles from the megakaryocytes is performed in a culture having a viability of about 45%. In embodiments, the isolation of megakaryocyte-derived extracellular vesicles from the megakaryocytes is performed in a culture having a viability of about 40%. In embodiments, the isolation of megakaryocyte-derived extracellular vesicles from the megakaryocytes is performed in a culture having a viability of about 30%.

In embodiments, the isolation of megakaryocyte-derived extracellular vesicles from the megakaryocytes is performed in a culture having a viability greater than about 25%. In embodiments, the isolation of megakaryocyte-derived extracellular vesicles from the megakaryocytes is performed in a culture having a viability greater than about 30%. In embodiments, the isolation of megakaryocyte-derived extracellular vesicles from the megakaryocytes is performed in a culture having a viability greater than about 35%. In embodiments, the isolation of megakaryocyte-derived extracellular vesicles from the megakaryocytes is performed in a culture having a viability greater than about 40%. In embodiments, the isolation of megakaryocyte-derived extracellular vesicles from the megakaryocytes is performed in a culture having a viability greater than about 45%. In embodiments, the isolation of megakaryocyte-derived extracellular vesicles from the megakaryocytes is performed in a culture having a viability greater than about 50%. In embodiments, the isolation of megakaryocyte-derived extracellular vesicles from the megakaryocytes is performed in a culture having a viability greater than about 55%. In embodiments, the isolation of megakaryocyte-derived extracellular vesicles from the megakaryocytes is performed in a culture having a viability greater than about 60%. In embodiments, the isolation of megakaryocyte-derived extracellular vesicles from the megakaryocytes is performed in a culture having a viability greater than about 65%. In embodiments, the isolation of megakaryocyte-derived extracellular vesicles from the megakaryocytes is performed in a culture having a viability greater than about 70%. In embodiments, the isolation of megakaryocyte-derived extracellular vesicles from the megakaryocytes is performed in a culture having a viability greater than about 75%. In embodiments, the isolation of megakaryocyte-derived extracellular vesicles from the megakaryocytes is performed in a culture having a viability greater than about 80%. In embodiments, the isolation of megakaryocyte-derived extracellular vesicles from the megakaryocytes is performed in a culture having a viability greater than about 85%. In embodiments, the isolation of megakaryocyte-derived extracellular vesicles from the megakaryocytes is performed in a culture having a viability greater than about 90%. In embodiments, the isolation of megakaryocyte-derived extracellular vesicles from the megakaryocytes is performed in a culture having a viability greater than about 95%.

In embodiments, the presently disclosed methods yield more megakaryocyte-derived extracellular vesicles than a comparable method with megakaryocytes in a culture having a viability greater than about 15%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% or more.

In embodiments, the presently disclosed methods yield more megakaryocyte-derived extracellular vesicles than a comparable method with megakaryocytes in a culture having greater than 30% viability, greater than 40% viability, greater than 50% viability, greater than 60% viability, greater than 70% viability, greater than 80% viability, greater than 90% viability, or greater than 95% viability or more.
wherein the viability is about 50% or less, e.g. about 50%, or about 45%, or about 40%, or about 30%.

### Isolation Post-Differentiation

In embodiments, the isolation of megakaryocyte-derived extracellular vesicles from the megakaryocytes occurs at least or at about 5 days, about 6 days, about 7 days, about 8 days, about 9 days, about 10, days, about 11 days, about 12 days, about 13 days, about 14 days, about 15 days, about 16 days, about 17 days, about 18, days, about 19 days, about 20 days, about 21 days, about 22 days, about 23 days, about 24 days, about 25 days, about 26 days, about 27 days, about 28, days, about 29 days, about 30 days or more after commencement of the differentiation of human pluripotent stem cells to megakaryocytes in culture.

In embodiments, the isolation of megakaryocyte-derived extracellular vesicles from the megakaryocytes occurs at about 7 days or at least at about 7 days after commencement of the differentiation of the differentiation of human pluripotent stem cells to megakaryocytes in culture. In embodiments, the isolation of megakaryocyte-derived extracellular vesicles from the megakaryocytes occurs at about 8 days or at least at about 8 days after commencement of the differentiation of the differentiation of human pluripotent stem cells to megakaryocytes in culture. In embodiments, the isolation of megakaryocyte-derived extracellular vesicles from the megakaryocytes occurs at about 9 days or at least at about 9 days after commencement of the differentiation of the differentiation of human pluripotent stem cells to megakaryocytes in culture. In embodiments, the isolation of megakaryocyte-derived extracellular vesicles from the megakaryocytes occurs at about 10 days or at least at about 10 days after commencement of the differentiation of the differentiation of human pluripotent stem cells to megakaryocytes in culture. In embodiments, the isolation of megakaryocyte-derived extracellular vesicles from the megakaryocytes occurs at about 11 days or at least at about 11 days after commencement of the differentiation of the differentiation of human pluripotent stem cells to megakaryocytes in culture. In embodiments, the isolation of megakaryocyte-derived extracellular vesicles from the megakaryocytes occurs at about 12 days or at least at about 12 days after commencement of the differentiation of the differentiation of human pluripotent stem cells to megakaryocytes in culture. In embodiments, the isolation of megakaryocyte-derived extracellular vesicles from the megakaryocytes occurs at about 13 days or at least at about 13 days after commencement of the differentiation of the differentiation of human pluripotent stem cells to megakaryocytes in culture. In embodiments, the isolation of megakaryocyte-derived extracellular vesicles from the megakaryocytes occurs at about 14 days or at least at about 14 days after commencement of the differentiation of the differentiation of human pluripotent stem cells to megakaryocytes in culture. In embodiments, the isolation of megakaryocyte-derived extracellular vesicles from the megakaryocytes occurs at about 15 days or at least at about 15 days after commencement of the differentiation of the differentiation of human pluripotent stem cells to megakaryocytes in culture. In embodiments, the isolation of megakaryocyte-derived extracellular vesicles from the megakaryocytes occurs at about 16 days or at least at about 16 days after commencement of the differentiation of the differentiation of human pluripotent stem cells to megakaryocytes in culture. In embodiments, the isolation of megakaryocyte-derived extracellular vesicles from the megakaryocytes occurs at about 17 days or at least at about 17 days after commencement of the differentiation of the differentiation of human pluripotent stem cells to megakaryocytes in culture. In embodiments, the isolation of megakaryocyte-derived extracellular vesicles from the megakaryocytes occurs at about 18 days or at least at about 18 days after commencement of the differentiation of the differentiation of human pluripotent stem cells to megakaryocytes in culture. In embodiments, the isolation of megakaryocyte-derived extracellular vesicles from the megakaryocytes occurs at about 19 days or at least at about 19 days after commencement of the differentiation of the differentiation of human pluripotent stem cells to megakaryocytes in culture. In embodiments, the isolation of megakaryocyte-derived extracellular vesicles from the megakaryocytes occurs at about 20 days or at least at about 20 days after commencement of the differentiation of the differentiation of human pluripotent stem cells to megakaryocytes in culture. In embodiments, the isolation of megakaryocyte-derived extracellular vesicles from the megakaryocytes occurs at about 21 days or at least at about 21 days after commencement of the differentiation of the differentiation of human pluripotent stem cells to megakaryocytes in culture. In embodiments, the isolation of megakaryocyte-derived extracellular vesicles from the megakaryocytes occurs at about 22 days or at least at about 22 days after commencement of the differentiation of the differentiation of human pluripotent stem cells to megakaryocytes in culture. In embodiments, the isolation of megakaryocyte-derived extracellular vesicles from the megakaryocytes occurs at about 23 days or at least at about 23 days after commencement of the differentiation of the differentiation of human pluripotent stem cells to megakaryocytes in culture. In embodiments, the isolation of megakaryocyte-derived extracellular vesicles from the megakaryocytes occurs at about 24 days or at least at about 24 days after commencement of the differentiation of the differentiation of human pluripotent stem cells to megakaryocytes in culture. In embodiments, the isolation of megakaryocyte-derived extracellular vesicles from the megakaryocytes occurs at about 25 days or at least at about 25 days after commencement of the differentiation of the differentiation of human pluripotent stem cells to megakaryocytes in culture.

In embodiments, the presently disclosed methods yield more megakaryocyte-derived extracellular vesicles than a comparable method in which isolation occurs at least less than 30 days, less than 29 days, less than 28 days, less than 27 days, less than 26 days, less than 25 days, less than 24 days, less than 23 days, less than 22 days, less than 21 days, less than 20 days, less than 19 days, less than 18 days, less than 17 days, less than 16 days, less than 15 days, less than 14 days, less than 13 days, less than 12 days, less than 11 days, less than 10 days, less than 9 days, less than 8 days, less than 7 days, less than 6 days, less than 5 days, less than 4 days, less than 3 days, less than 2 days, less than 1 days after commencement of the differentiation, or the same day of the commencement of the differentiation.

In embodiments, the presently disclosed methods yield more megakaryocyte-derived extracellular vesicles than a comparable method in which isolation occurs at least less than 20 days after commencement of the differentiation. In embodiments, the presently disclosed methods yield more megakaryocyte-derived extracellular vesicles than a comparable method in which isolation occurs at least less than 19 days after commencement of the differentiation. In embodiments, the presently disclosed methods yield more megakaryocyte-derived extracellular vesicles than a comparable method in which isolation occurs at least less than 18 days after commencement of the differentiation. In embodiments, the presently disclosed methods yield more megakaryocyte-derived extracellular vesicles than a comparable method in which isolation occurs at least less than 17 days after commencement of the differentiation. In embodiments, the presently disclosed methods yield more megakaryocyte-derived extracellular vesicles than a comparable method in which isolation occurs at least less than 16 days after commencement of the differentiation. In embodiments, the presently disclosed methods yield more megakaryocyte-derived extracellular vesicles than a comparable method in which isolation occurs at least less than 15 days after commencement of the differentiation. In embodiments, the presently disclosed methods yield more megakaryocyte-derived extracellular vesicles than a comparable method in which isolation occurs at 14 days after commencement of the differentiation. In embodiments, the presently disclosed methods yield more megakaryocyte-derived extracellular vesicles than a comparable method in which isolation occurs at 13 days after commencement of the differentiation. In embodiments, the presently disclosed methods yield more megakaryocyte-derived extracellular vesicles than a comparable method in which isolation occurs at 12 days after commencement of the differentiation. In embodiments, the presently disclosed methods yield more megakaryocyte-derived extracellular vesicles than a comparable method in which isolation occurs at 11 days after commencement of the differentiation.

### Purity

In embodiments, the disclosed methods are substantially serum free. In embodiments, the methods are greater than 60% serum free. In embodiments, the methods are greater than 70% serum free. In embodiments, the methods are greater than 80% serum free. In embodiments, the methods are greater than 90% serum free.

In various embodiments, the disclosed methods comprise substantially purified megakaryocyte-derived extracellular vesicles. In embodiments, substantially purified is synonymous with biologically pure. In embodiments, the substantially purified megakaryocyte-derived extracellular vesicles are largely free to varying degrees from components which normally accompany it as found in its native state. “Isolate” denotes a degree of separation from original source or surroundings. In embodiments, the substantially purified megakaryocyte-derived extracellular vesicles are sufficiently free of other materials such that any impurities do not materially affect the biological properties of the megakaryocyte-derived extracellular vesicles or cause other adverse consequences. In embodiments, the substantially purified megakaryocyte-derived extracellular vesicles are sufficiently free of cellular material, viral material, or culture medium that may be needed for production. Purity and homogeneity are typically determined using biochemical techniques known in the art.

In one aspect, disclosed herein are methods for purifying a plurality of megakaryocyte-derived extracellular vesicles. The methods comprise (a) obtaining a material comprising a population of megakaryocytes in culture or the cell culture media thereof, and (b) filtering the material of step (a) thereby yielding predominantly CD41+ megakaryocyte-derived extracellular vesicles in the filtrate.

In embodiments, the megakaryocyte-derived extracellular vesicles are purified using by using tangential flow filtration. Tangential flow filtration (TFF) is a rapid and efficient method for separation and purification of biomolecules well known in the art. In embodiments, the tangential flow filtration is peristaltic. In embodiments, the tangential flow filtration is non-peristaltic.

In embodiments, the filtering is achieved by using a low-pass acoustic filter, using a counterflow centrifugation elutriation, using a centrifugal pump that is based on magnetic levitation principles.

In embodiments, the disclosed methods yield a plurality of megakaryocyte-derived extracellular vesicles that are substantially intact. In embodiments, the disclosed methods yield a plurality of megakaryocyte-derived extracellular vesicles that are suitable for cargo loading.

In embodiments, the megakaryocyte-derived extracellular vesicles are purified using size exclusion filtration. In embodiments, the filter has a pore size of about 650 nm. In embodiments, the megakaryocyte-derived extracellular vesicles are purified using size exclusion filtration. In embodiments, the filter has a pore size ranging from about 50 nm to about 600 nm. In embodiments, the filter has a pore size of at least 50 nm. In embodiments, the filter has a pore size of about 600 nm.

In embodiments, the disclosed methods comprise megakaryocyte-derived extracellular vesicles that are substantially of a diameter in the range between about 100 nm to about 600 nm. In embodiments, the megakaryocyte-derived extracellular vesicles are substantially of a diameter in the range between about 30 nm to about 100 nm. In embodiments, the megakaryocyte-derived extracellular vesicles are substantially of a diameter in the range between about 100 nm to about 300 nm.

In further embodiments, about 90% or more, or about 95% or more, or about 97% or more, or about 99% or more of the megakaryocyte-derived extracellular vesicles are of a diameter of between about 100 nm and about 600 nm. In embodiments, about 90% or more, or about 95% or more, or about 97% or more, or about 99% or more of the megakaryocyte-derived extracellular vesicles are of a diameter of between about 100 nm and about 300 nm.

In embodiments, the disclosed methods to generate megakaryocyte-derived extracellular vesicles are standardized to enable large-scale production.

In embodiments, a cell culture process used for the presently disclosed methods is adapted to produce allogeneic megakaryocyte-derived extracellular vesicles from primary human peripheral blood CD34+ human surface cell markers (HSCs). In embodiments, the megakaryocyte-derived extracellular vesicles are produced by a method comprising obtaining primary human peripheral blood CD34+ HSCs sourced from a commercial supplier and transitioning from a stem cell maintenance medium to an HSC expansion medium. In embodiments, the megakaryocyte-derived extracellular vesicles are produced by a method comprising obtaining primary human cord blood CD34+ HSCs. In embodiments, the megakaryocyte-derived extracellular vesicles are produced by a method comprising obtaining primary human bone marrow CD34+ HSCs. In embodiments, the method further involves placing HSC cultures in a megakaryocyte differentiation medium and collecting megakaryocyte-derived extracellular vesicles from culture supernatant. Accordingly, in embodiments, the present megakaryocyte-derived extracellular vesicles are produced from starting CD34+ HSCs.

In embodiments, the megakaryocyte differentiation is confirmed by biomarker expression and/or presence of one or more of CD41, CD61, CD42b, megakaryocyte-specific cytoskeletal proteins β1-tubulin, alpha granule components (e.g. platelet factor 4 and von Willebrand Factor), secretory granules, and ultrastructural characteristics (e.g. invaginated membrane system, dense tubular system, multivesicular bodies).

In embodiments, the megakaryocytes yield between about 500 to about 1500 megakaryocyte-derived extracellular vesicles/cell, which are between about 100 and about 600 nm in diameter (average about 200 nm), DNA-, and CD41+. In embodiments, the megakaryocyte-derived extracellular vesicles are further isolated/concentrated by tangential flow filtration and packaged at targeted concentrations of about 1.5×10⁸ megakaryocyte-derived extracellular vesicles/mL. In embodiments, the megakaryocyte-derived extracellular vesicles exhibit robust expression and/or presence of megakaryocyte and platelet-specific biomarkers, RNA, and cytosolic proteins.

In embodiments, nanoparticle analysis, electron microscopy, flow cytometry, and/or western blots are used to confirm biomarker expression and/or presence and composition of megakaryocyte-derived extracellular vesicles.

In embodiments, the megakaryocyte-derived extracellular vesicles are isolated from megakaryocytes, which are generated in the absence of added erythropoietin. In embodiments, the megakaryocyte-derived extracellular vesicles are isolated from megakaryocytes which are generated in the presence of added thrombopoietin.

In embodiments, the megakaryocyte-derived extracellular vesicles are substantially free of nucleic acids. In embodiments, the megakaryocyte-derived extracellular vesicles are substantially free of autologous nucleic acids. In embodiments, the megakaryocyte-derived extracellular vesicles are substantially free of RNA. In embodiments, the megakaryocyte-derived extracellular vesicles comprise nucleic acids. In embodiments, the megakaryocyte-derived extracellular vesicles comprise autologous nucleic acids. In embodiments, the megakaryocyte-derived extracellular vesicles comprise autologous RNA. Non-limiting examples of RNA include rRNA, siRNA, microRNA, regulating RNA, and/or non-coding and coding RNA. In embodiments, the megakaryocyte-derived extracellular vesicles are substantially free of RNA from the cell from which the vesicles are derived. In non-limiting examples, the megakaryocyte-derived extracellular vesicles do not contain RNA due to the method of preparing the vesicles and/or due to the use of RNase to remove native RNAs.

In embodiments, the megakaryocyte-derived extracellular vesicles are substantially free of autologous DNA. In embodiments, the megakaryocyte-derived extracellular vesicles are substantially free of DNA from the cell from which the vesicles are derived. In non-limiting examples, the megakaryocyte-derived extracellular vesicles do not contain DNA due to the method of preparing the vesicles and/or due to the use of DNase to remove native DNAs. In embodiments, the megakaryocyte-derived extracellular vesicles are substantially free of one or more of: (a) megakaryocytes, (b) megakaryocyte-derived platelets, and (c) extracellular vesicles derived from platelets.

In embodiments, frozen granulocyte colony-stimulating factor (G-CSF) mobilized human peripheral blood CD34+ cells are obtained and cultured to megakaryocytes before subsequently enriching CD41+ cells (megakaryocytes) prior to culturing, and then measuring the CD41 expression and/or presence and concentration of megakaryocyte-derived extracellular vesicles in the cell culture by flow cytometer or nanoparticle analysis. In embodiments, the megakaryocyte-derived extracellular vesicles are generated by a series of centrifugations, e.g. at escalating speeds/force. In embodiments, the megakaryocyte-derived extracellular vesicles are generated by: (a) removing cells from culture medium at, e.g., about 150×g centrifugation for, e.g., about 10 min; (b) removing platelet-like particles (PLPs) and cell debris by centrifugation at, e.g., about 1000×g for, e.g., about 10 min; and (c) enriching the megakaryocyte-derived extracellular vesicles from the supernatant by ultracentrifugation at, e.g., about 25,000 rpm (38000×g) for, e.g., about 1 hour at, e.g., about 4° C.

In embodiments, a multi-phase culture process with differing pH and pO₂ or pCO₂ and different cytokine cocktails is used to greatly increase megakaryocyte production.

In embodiments, the megakaryocytes are generated by: (a) culturing CD34+ HSCs with a molecular signal/factor/cytokine cocktail that promotes megakaryocyte progenitor production; and (b) shifting cells to different conditions to expand mature megakaryocytes from progenitors. In embodiments, commercial media is used. In embodiments, serum-free media is used. In embodiments, pH is shifted to increase megakaryocyte production. In embodiments, percent CO₂ is shifted to increase megakaryocyte production. In embodiments, the identity of the molecular signals/factors/cytokines is altered to increase megakaryocyte production. In embodiments, the molecular signal/factor/cytokine cocktail contains one or more of TPO, GM-CSF, IL-3, IL-6, IL-11, SCF, Flt3L, IL-9, and the like.

In embodiments, the present production methods further involve the step of characterizing the resultant megakaryocyte-derived extracellular vesicles for one or more of CD54, CD18, CD43, CD11b, CD62P, CD41, CD61, CD21, CD51, CLEC-2, LAMP-1 (CD107a), CD63, CD42b, CD9, CD31, CD47, CD147, CD32a, and GPVI. e.g., without limitation by nanoparticle analysis, electron microscopy, flow cytometry, and/or western blot analysis. In embodiments, the present production methods further involve the step of characterizing the resultant megakaryocyte-derived extracellular vesicles for phosphatidylserine, e.g., without limitation by testing for Annexin V, e.g., without limitation by nanoparticle analysis, electron microscopy, flow cytometry, and/or western blot analysis.

In embodiments, the present methods to generate megakaryocyte-derived extracellular vesicles inter-batch/donor variability is of less than about 20%, or less than about 15%, or less than about 10%, or less than about 5%. In embodiments, methods to generate megakaryocyte-derived extracellular vesicles are developed such that inter-batch/donor variability is less than 12.5%. In embodiments, methods to generate megakaryocyte-derived extracellular vesicles are developed such that inter-batch/donor variability is less than 10%. In embodiments, methods to generate megakaryocyte-derived extracellular vesicles are developed such that inter-batch/donor variability is less than 7.5%. In embodiments, methods to generate megakaryocyte-derived extracellular vesicles are developed such that inter-batch/donor variability is less than 5%. In embodiments, methods to generate megakaryocyte-derived extracellular vesicles are developed such that inter-batch/donor variability is less than 2.5%.

In embodiments, the population comprises about 1×10⁷ or more, about 1.5×10⁷ or more, about 5×10⁷ or more, 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

In embodiments, the megakaryocyte-derived extracellular vesicles are isolated as a population. In embodiments, the population of megakaryocyte-derived extracellular vesicles is substantially homogenous.

In embodiments, substantially all of the megakaryocyte-derived extracellular vesicles in the population comprise CD41. In embodiments, about 15% to about 90%, about 30% to about 80%, or about 50% to about 70% of the megakaryocyte-derived extracellular vesicles in the population comprise CD41. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD41.

In embodiments, the megakaryocyte-derived extracellular vesicles comprise CD41. In embodiments, the megakaryocyte-derived extracellular vesicles comprise greater than about 40% CD41.

In embodiments, the disclosed methods yield a preparation of megakaryocyte-derived extracellular vesicles that are devoid of CD41 negative (CD41−) vesicles.

In embodiments, substantially all of the megakaryocyte-derived extracellular vesicles in the population comprise CD54. In embodiments, about 0% to about 5%, about 0% to about 10%, about 15% to about 90%, about 30% to about 80%, or about 50% to about 70% of the megakaryocyte-derived extracellular vesicles in the population comprise CD54. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD54. In embodiments, less than about 30%, less than about 25%, less than about 20%, less than about 15%, less than about 10%, less than about 5%, or less than about 1% of the megakaryocyte-derived extracellular vesicles in the population comprise CD54. In embodiments, all of the megakaryocyte-derived extracellular vesicles in the population are free of, or substantially free of CD54.

In embodiments, substantially all of the megakaryocyte-derived extracellular vesicles in the population comprise CD18. In embodiments, about 0% to about 5%, about 0% to about 10%, about 15% to about 90%, about 30% to about 80%, or about 50% to about 70% of the megakaryocyte-derived extracellular vesicles in the population comprise CD18. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD18. In embodiments, less than about 30%, less than about 25%, less than about 20%, less than about 15%, less than about 10%, less than about 5%, or less than about 1% of the megakaryocyte-derived extracellular vesicles in the population comprise CD18. In embodiments, all of the megakaryocyte-derived extracellular vesicles in the population are free of, or substantially free of CD18.

In embodiments, substantially all of the megakaryocyte-derived extracellular vesicles in the population comprise CD43. In embodiments, about 1% to about 30%, about 1% to about 25%, about 1% to about 20%, or about 1% to about 15%, about 0% to about 5% or about 0% to about 10% of the megakaryocyte-derived extracellular vesicles in the population comprise CD43. In embodiments, less than about 30%, less than about 25%, less than about 20%, less than about 15%, less than about 10%, less than about 5%, or less than about 1% of the megakaryocyte-derived extracellular vesicles in the population comprise CD43. In embodiments, all of the megakaryocyte-derived extracellular vesicles in the population are free of, or substantially free of CD43.

In embodiments, substantially all of the megakaryocyte-derived extracellular vesicles in the population comprise CD11 b. In embodiments, about 0% to about 5%, about 0% to about 10%, about 1% to about 50%, about 5% to about 40%, or about 10% to about 35% of the megakaryocyte-derived extracellular vesicles in the population comprise CD11 b. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD11 b. In embodiments, all of the megakaryocyte-derived extracellular vesicles in the population are free of, or substantially free of CD11 b.

In embodiments, substantially all of the megakaryocyte-derived extracellular vesicles in the population comprise CD62P. In embodiments, about 0% to about 40%, about 0% to about 30%, about 0% to about 20%, about 0% to about 10%, or about 0% to about 5%, of the megakaryocyte-derived extracellular vesicles in the population comprise CD62P. In embodiments, less than about 40%, less than about 30%, less than about 20%, less than about 10% of the megakaryocyte-derived extracellular vesicles in the population comprise CD62P. In embodiments, all of the megakaryocyte-derived extracellular vesicles in the population are free of, or substantially free of CD62P.

In embodiments, substantially all of the megakaryocyte-derived extracellular vesicles in the population comprise CD61. In embodiments, about 40% to about 100%, about 60% to about 100%, or about 85% to about 10% of the megakaryocyte-derived extracellular vesicles in the population comprise CD61. In embodiments, greater than about 40%, greater than about 50%, greater than about 60%, greater than about 70%, greater than about 80%, greater than about 85%, greater than about 90%, or greater than about 95%, or greater than about 99% of the megakaryocyte-derived extracellular vesicles in the population comprise CD61.

In embodiments, substantially all of the megakaryocyte-derived extracellular vesicles in the population comprise CD21. In embodiments, about 0% to about 10%, about 0% to about 5%, about 15% to about 90%, about 30% to about 80%, or about 50% to about 70% of the megakaryocyte-derived extracellular vesicles in the population comprise CD21. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD21. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD21. In embodiments, all of the megakaryocyte-derived extracellular vesicles in the population are free of, or substantially free of CD21.

In embodiments, substantially all of the megakaryocyte-derived extracellular vesicles in the population comprise CD51. In embodiments, about 0% to about 10%, about 0% to about 5%, about 15% to about 90%, about 30% to about 80%, or about 50% to about 70% of the megakaryocyte-derived extracellular vesicles in the population comprise CD51. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD51. In embodiments, less than about 50%, less than about 45%, less than about 40%, less than about 35%, less than about 30%, less than about 25%, less than about 20%, less than about 15%, less than about 10%, or less than about 5% of the megakaryocyte-derived extracellular vesicles in the population comprise CD51. In embodiments, all of the megakaryocyte-derived extracellular vesicles in the population are free of, or substantially free of CD51.

In embodiments, substantially all of the megakaryocyte-derived extracellular vesicles in the population comprise CLEC-2. In embodiments, about 0% to about 10%, about 0% to about 5%, or about 0% to about 12% of the megakaryocyte-derived extracellular vesicles in the population comprise CLEC-2. In embodiments, less than about 10%, less than about 5%, or less than about 2% of the megakaryocyte-derived extracellular vesicles in the population comprise CLEC-2. In embodiments, all of the megakaryocyte-derived extracellular vesicles in the population are free of, or substantially free of CLEC-2.

In embodiments, substantially all of the megakaryocyte-derived extracellular vesicles in the population comprise LAMP-1 (CD107a). In embodiments, about 0% to about 20%, about 1% to about 15%, about 2% to about 10%, about 0% to about 5%, or about 0% to about 5% of the megakaryocyte-derived extracellular vesicles in the population comprise LAMP-1 (CD107a). In embodiments, less than about 20%, less than about 15%, less than about 10%, or less than about 5% of the megakaryocyte-derived extracellular vesicles in the population comprise LAMP-1 (CD107a). In embodiments, all of the megakaryocyte-derived extracellular vesicles in the population are free of, or substantially free of LAMP-1 (CD107a).

In embodiments, less than about 20%, less than about 15%, less than about 10%, or less than about 5% of a population of CD41+ megakaryocyte-derived extracellular vesicles comprise LAMP-1 (CD107a).

In embodiments, the megakaryocyte-derived extracellular vesicles in the population are substantially free of DRAQ5. In embodiments, about 0% to about 20%, about 0% to about 15%, about 0% to about 10%, or about 0% to about 5% of the megakaryocyte-derived extracellular vesicles in the population comprise DRAQ5. In embodiments, less than about 20%, less than about 15%, less than about 10%, or less than about 5% of the megakaryocyte-derived extracellular vesicles in the population comprise DRAQ5.

In embodiments, substantially all of the megakaryocyte-derived extracellular vesicles in the population comprise CD63. In embodiments, about 1% to about 20%, about 1% to about 15%, or about 1% to about 10% of the megakaryocyte-derived extracellular vesicles in the population comprise CD63. In embodiments, less than about 20%, less than about 15%, less than about 10%, less than about 5%, or less than about 1% of the megakaryocyte-derived extracellular vesicles in the population comprise CD63. In embodiments, all of the megakaryocyte-derived extracellular vesicles in the population are free of, or substantially free of CD63.

In embodiments, substantially all of the megakaryocyte-derived extracellular vesicles in the population comprise CD42b. In embodiments, about 0% to about 20%, about 0% to about 15%, about 0% to about 10%, or about 0% to about 5% of the megakaryocyte-derived extracellular vesicles in the population comprise CD42b. In embodiments, less than about 20%, less than about 15%, less than about 10%, less than about 5%, or less than about 1% of the megakaryocyte-derived extracellular vesicles in the population comprise CD42b. In embodiments, all of the megakaryocyte-derived extracellular vesicles in the population are free of, or substantially free of CD42b

In embodiments, substantially all of the megakaryocyte-derived extracellular vesicles in the population comprise CD9. In embodiments, about 40% to about 100%, about 50% to about 80%, or about 60% to about 70% of the megakaryocyte-derived extracellular vesicles in the population comprise CD9. In embodiments, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise CD9.

In embodiments, substantially all of the megakaryocyte-derived extracellular vesicles in the population comprise CD31. In embodiments, about 1% to about 30%, about 1% to about 25%, about 1% to about 20%, or about 1% to about 15% of the megakaryocyte-derived extracellular vesicles in the population comprise CD31. In embodiments, less than about 30%, less than about 25%, less than about 20%, less than about 15%, less than about 10%, less than about 5%, or less than about 1% of the megakaryocyte-derived extracellular vesicles in the population comprise CD31. In embodiments, all of the megakaryocyte-derived extracellular vesicles in the population are free of, or substantially free of CD31.

In embodiments, substantially all of the megakaryocyte-derived extracellular vesicles in the population comprise CD47. In embodiments, about 1% to about 40%, about 1% to about 35%, about 1% to about 20%, about 20% to about 30%, about 30% to about 40%, or about 1% to about 15% of the megakaryocyte-derived extracellular vesicles in the population comprise CD47. In embodiments, less than about 40%, less than about 30%, less than about 25%, less than about 20%, less than about 15%, less than about 10%, less than about 5%, or less than about 1% of the megakaryocyte-derived extracellular vesicles in the population comprise CD47. In embodiments, all of the megakaryocyte-derived extracellular vesicles in the population are free of, or substantially free of CD47.

In embodiments, substantially all of the megakaryocyte-derived extracellular vesicles in the population comprise CD147. In embodiments, about 1% to about 30%, about 1% to about 25%, about 1% to about 20%, about 20% to about 30%, or about 1% to about 15% of the megakaryocyte-derived extracellular vesicles in the population comprise CD147. In embodiments, less than about 30%, less than about 25%, less than about 20%, less than about 15%, less than about 10%, less than about 5%, or less than about 1% of the megakaryocyte-derived extracellular vesicles in the population comprise CD147. In embodiments, all of the megakaryocyte-derived extracellular vesicles in the population are free of, or substantially free of CD147.

In embodiments, substantially all of the megakaryocyte-derived extracellular vesicles in the population comprise CD32a. In embodiments, about 0% to about 20%, about 1% to about 15%, or about 1% to about 10% of the megakaryocyte-derived extracellular vesicles in the population comprise CD32a. In embodiments, less than about 20%, less than about 15%, less than about 10%, less than about 5%, or less than about 1% of the megakaryocyte-derived extracellular vesicles in the population comprise CD32a. In embodiments, all of the megakaryocyte-derived extracellular vesicles in the population are free of, or substantially free of CD32a.

In embodiments, substantially all of the megakaryocyte-derived extracellular vesicles in the population comprise GVPI. In embodiments, about 0% to about 5%, about 0% to about 10%, about 0% to about 30%, about 0% to about 15%, or about 0% to about 10% of the megakaryocyte-derived extracellular vesicles in the population comprise GVPI. In embodiments, less than about 30%, less than about 25%, less than about 20%, less than about 15%, less than about 10%, less than about 5%, or less than about 1% of the megakaryocyte-derived extracellular vesicles in the population comprise GVPI. In embodiments, all of the megakaryocyte-derived extracellular vesicles in the population are free of, or substantially free of GVPI.

In embodiments, substantially all of the megakaryocyte-derived extracellular vesicles in the population comprise phosphatidylserine. In embodiments, about 15% to about 90%, about 30% to about 80%, or about 50% to about 70% of the megakaryocyte-derived extracellular vesicles in the population comprise phosphatidylserine. In embodiments, greater than about 20%, greater than about 30%, greater than about 40%, greater than about 50%, greater than about 60%, greater than about 70%, greater than about 80%, greater than about 90%, or greater than about 95%, or greater than about 99% of the megakaryocyte-derived extracellular vesicles in the population comprise phosphatidylserine. In embodiments, less than about 30%, less than about 25%, less than about 20%, less than about 15%, less than about 10%, less than about 5%, or less than about 1% of the megakaryocyte-derived extracellular vesicles in the population comprise GVPI. In embodiments, all of the megakaryocyte-derived extracellular vesicles in the population are free of, or substantially free of phosphatidylserine.

### Cargo Loading

In one aspect, the present invention relates to methods of loading cargo in a plurality of megakaryocyte-derived extracellular vesicles. In embodiments, the presently disclosed methods comprise (a) obtaining a plurality of megakaryocyte-derived extracellular vesicles; (b) contacting the plurality of megakaryocyte-derived extracellular vesicles with a cargo of interest; and (c) applying an electrical pulse for a period of time, thereby permitting the cargo to pass into the lumen of the megakaryocyte-derived extracellular vesicles, wherein the electrical pulse is applied for about 5 to about 25 milliseconds and the electrical pulse is applied about 5 to about 25 times. In embodiments, the electrical pulse is applied for about 10 to about 20 milliseconds and the electrical pulse is applied about 10 to about 20 times. In embodiments, the electrical pulse is applied for about 5 to about 25 milliseconds and the electrical pulse is applied about 5 to about 25 times. In embodiments, the electrical pulse is applied for about 10 to about 20 milliseconds and the electrical pulse is applied about 5 to about 25 times.

In another aspect, the present invention relates to methods of loading cargo in a plurality of megakaryocyte-derived extracellular vesicles. The methods comprise (a) obtaining a plurality of megakaryocyte-derived extracellular vesicles; (b) contacting the plurality of megakaryocyte-derived extracellular vesicles with a cargo of interest; and (c) applying an electrical pulse for a period of time, thereby permitting the cargo to pass into the lumen of the megakaryocyte-derived extracellular vesicles, wherein the electrical pulse is applied for at least about 15 milliseconds and the electrical pulse is applied for at least about 10 times. In embodiments, the electrical pulse is applied for about 15 milliseconds and the electrical pulse is applied about 10 times. In embodiments, the electrical pulse is applied for about 15 milliseconds. In embodiments, the electrical pulse is applied for about 10 times.

In embodiments, the electrical pulse is applied for about 1 to about 60, milliseconds, about 5 to about 55 milliseconds, about 5 to about 50, milliseconds, about 5 to about 45 milliseconds, about 5 to about 40, milliseconds, about 5 to about 35 milliseconds, about 5 to about 30, milliseconds, about 5 to about 25 milliseconds, about 5 to about 15, milliseconds, about 5 to about 10 milliseconds, about 10 to about 55 milliseconds, about 10 to about 50 milliseconds, about 10 to about 45 milliseconds, about 10 to about 40 milliseconds, about 10 to about 35 milliseconds, about 10 to about 30, milliseconds, about 10 to about 25 milliseconds, about 10 to about 15 milliseconds, or any range there in between. In embodiments, the electrical pulse is applied for about 1 milliseconds, about 2 milliseconds, about 3 milliseconds, about 4 milliseconds, about 5 milliseconds, about 6 milliseconds, about 7 milliseconds, about 8 milliseconds, about 9 milliseconds, about 10 milliseconds, about 11 milliseconds, about 12 milliseconds, about 13 milliseconds, about 14 milliseconds, about 15 milliseconds, about 16 milliseconds, about 17 milliseconds, about 18 milliseconds, about 19 milliseconds, about 20 milliseconds, about 21 milliseconds, about 22 milliseconds, about 23 milliseconds, about 24 milliseconds, about 25 milliseconds, about 26 milliseconds, about 27 milliseconds, about 28 milliseconds, about 29 milliseconds, about 30 milliseconds, about 31 milliseconds, about 32 milliseconds, about 33 milliseconds, about 34 milliseconds, about 35 milliseconds, about 36 milliseconds, about 37 milliseconds, about 38 milliseconds, about 39 milliseconds, about 40 milliseconds, about 41 milliseconds, about 42 milliseconds, about 43 milliseconds, about 44 milliseconds, about 45 milliseconds, about 46 milliseconds, about 47 milliseconds, about 48 milliseconds, about 49 milliseconds, about 50 milliseconds, about 51 milliseconds, about 52 milliseconds, about 53 milliseconds, about 54 milliseconds, about 55 milliseconds, about 56 milliseconds, about 57 milliseconds, about 58 milliseconds, about 59 milliseconds, about 60 milliseconds, or more.

In embodiments, the electrical pulse is applied for more than about 60 milliseconds, more than about 65 milliseconds, more than about 70 milliseconds, more than about 75 milliseconds, more than about 80 milliseconds, more than about 85 milliseconds, more than about 90 milliseconds, more than about 95 milliseconds, more than about 100 milliseconds, or more.

In embodiments, the electrical pulse is applied about 1 to about 40 times, about 2 to about 35 times, about 5 to about 30 times, about 5 to about 25 times, about 5 to about 20 times, about 5 to about 15 times, about 5 to about 10 times, about 8 to about 30 times, about 8 to about 25 times, about 10 to about 20 times, about 10 to about 15 times, about 10 to about 14 times, about 10 to about 13 times, about 10 to about 12 times, about 10 to about 11 times, about 8 to about 10 times, about 8 to about 15 times, about 8 to about 14 times, about 8 to about 13 times, about 8 to about 12 times, about 8 to about 11 times, about 8 to about 10 times, or any range there in between.

In embodiments, the electrical pulse is applied about 1 time or more than about 1 time, about 2 times or more than about 2 times, about 3 times or more than about 3 times, about 4 times or more than about 4 times, about 5 times or more than about 5 times, about 6 times or more than about 6 times, about 7 times or more than about 7 times, about 8 times or more than about 8 times, about 9 times or more than about 9 times, about 10 times or more than about 10 times, about 11 times or more than about 11 times, about 12 times or more than about 12 times, about 13 times or more than about 13 times, about 14 times or more than about 14 times, about 15 times or more than about 15 times, about 16 times or more than about 16 times, about 17 times or more than about 17 times, about 18 times or more than about 18 times, about 19 times or more than about 19 times, about 20 times or more than about 20 times, about 21 times or more than about 21 times, about 22 times or more than about 22 times, about 23 times or more than about 23 times, about 24 times or more than about 24 times, about 25 times or more than about 25 times, about 26 times or more than about 26 times, about 27 times or more than about 27 times, about 28 times or more than about 28 times, about 29 times or more than about 29 times, about 30 times or more than about 30 times, about 31 times or more than about 31 times, about 32 times or more than about 32 times, about 33 times or more than about 33 times, about 34 times or more than about 34 times, about 35 times or more than about 35 times, about 36 times or more than about 36 times, about 37 times or more than about 37 times, about 38 times or more than about 38 times, about 39 times or more than about 39 times, about 40 times or more than about 40 times, or more.

### Methods of Treatment Using Megakaryocyte-Derived Extracellular Vesicles

In various embodiments, the methods disclosed herein may be utilized for drug delivery and treatment of one or more genetic disorders.

### Infectious Disease

Infectious diseases are disorders that are caused by pathogenic microorganisms, such as bacteria, viruses, fungi, or parasites. Zoonotic diseases are infectious diseases of animals that can cause disease when transmitted to humans.

In another aspect, the present invention relates to a method for treating or preventing an infectious disease, comprising administering an effective amount of a composition (i.e. MkEvs) generated based on the methods disclosed herein.

In another aspect, the present invention relates to a method for treating or preventing an infectious disease, comprising administering an effective amount of a composition comprising a cell, which is contacted with a composition generated based on the methods disclosed herein in vitro.

In another aspect, the present invention relates to a method for treating or preventing an infectious disease, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In embodiments, the megakaryocyte-derived extracellular vesicles of the present compositions and methods are used to treat an infection caused by a virus (a viral infection) in a patient, wherein the viral infection is selected from one or more of: (a) the common cold, which mainly occurs due to rhinovirus, coronavirus, and adenovirus; (b) encephalitis and meningitis, resulting from enteroviruses and the herpes simplex virus (HSV), as well as West Nile Virus; (c) warts and skin infections, for which HPV and HSV are responsible; (d) gastroenteritis, caused by norovirus; (e) Zika; (f) AIDS/HIV; (g) Hepatitis; (h) polio; (i) influenza, including H1N1 swine flu; (j) Dengue fever; and (k) Ebola.

In embodiments, the megakaryocyte-derived extracellular vesicles are used to treat an infection caused by a bacterium (a bacterial infection) in a patient, wherein the bacterial infection is selected from one or more of: cholera, diphtheria, dysentery, bubonic plague, tuberculosis, typhoid, typhus, bacterial meningitis, otitis media, pneumonia, upper respiratory tract infection, gastritis, food poisoning, eye infection, sinusitis, urinary tract infection, skin infection, and sexually transmitted infection.

In embodiments, the megakaryocyte-derived extracellular vesicles are used to treat an infection caused by a fungus (a fungal infection) in a patient, wherein the fungal infection is selected from one or more of: valley fever (coccidioidomycosis), histoplasmosis, candidiasis, athlete's foot, ringworm, eye infection, and skin infection.

In embodiments, the megakaryocyte-derived extracellular vesicles are used to treat an infection caused by a parasite (a parasitic infection) in a patient, wherein the parasitic infection is selected from one or more of: malaria, sleeping sickness, amebiasis, trypanosomiasis, pediculosis, Chagas disease, cyclosporiasis, tapeworm infection, echinococcosis, foodborne disease, giardiasis, keratitis, leishmaniasis, onchocerciasis, trichinosis, waterborne disease, and zoonotic disease.

In embodiments, the megakaryocyte-derived extracellular vesicles are used to treat one or more symptoms associated with a coronavirus infection.

Coronaviruses (CoVs) are members of the family Coronaviridae, including betacoronavirus and alphacoronavirus-respiratory pathogens that have relatively recently become known to invade humans. The Coronaviridae family includes such betacoronavirus as Severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E. In embodiments, the present invention relates to the therapeutic use of the present megakaryocyte-derived extracellular vesicles for the treatment of one or more symptoms of infection with any of Severe acute respiratory syndrome coronavirus 2 (SARS-CoV-2), SARS-CoV, Middle East Respiratory Syndrome-Corona Virus (MERS-CoV), HCoV-HKU1, and HCoV-OC43. Alphacoronavirus includes, e.g., HCoV-NL63 and HCoV-229E.

Without wishing to be bound by theory, coronaviruses invade cells through utilization of their “spike” surface glycoprotein that is responsible for viral recognition of Angiotensin Converting Enzyme 2 (ACE2), a transmembrane receptor on mammalian hosts that facilitate viral entrance into host cells. (Zhou et al., A pneumonia outbreak associated with a new coronavirus of probable bat origin, Nature 2020).

Symptoms associated with coronavirus infections include, but are not limited to, fever, tiredness, dry cough, aches and pains, shortness of breath and other breathing difficulties, diarrhea, upper respiratory symptoms (e.g. sneezing, runny nose, nasal congestion, cough, sore throat), and/or pneumonia. In embodiments, the present compositions and methods are useful in treating or mitigating any of these symptoms.

In embodiments, the present invention relates to the therapeutic use of the present megakaryocyte-derived extracellular vesicles for the treatment of one or more symptoms of infection with SARS-CoV-2, including Coronavirus infection 2019 (COVID-19), caused by SARS-CoV-2 (e.g., 2019-nCoV).

In embodiments, the infectious disease is a coronavirus infection. In embodiments, the coronavirus infection is infection by a betacoronavirus or an alphacoronavirus, optionally wherein the betacoronavirus is selected from a SARS-CoV-2, SARS-CoV, MERS-CoV, HCoV-HKU1, and HCoV-OC43 or the alphacoronavirus is selected from a HCoV-NL63 and HCoV-229E. In embodiments, the coronavirus infection is infection by SARS-CoV-2. In embodiments, the infectious disease is COVID-19.

In embodiments, the infectious disease is an influenza infection, optionally selected from Type A, Type B, Type C, and Type D influenza.

In embodiments, the infectious disease is a retroviral infection, optionally selected from human immune deficiency (HIV) and simian immune deficiency (SIV).

In embodiments, the composition comprises megakaryocyte-derived extracellular vesicles, which comprise a nucleic acid molecule encoding a vaccine protein and/or an immunogenic antigen. In embodiments, the composition comprises megakaryocyte-derived extracellular vesicles, which comprise a nucleic acid molecule encoding a protein related to infectivity.

In embodiments, the vaccine protein is a betacoronavirus protein or an alphacoronavirus protein, optionally wherein the betacoronavirus protein is selected from a SARS-CoV-2, SARS-CoV, MERS-CoV, HCoV-HKU1, and HCoV-OC43 protein, or an antigenic fragment thereof or the alphacoronavirus protein is selected from a HCoV-NL63 and HCoV-229E protein, or an antigenic fragment thereof.

In embodiments, the SARS-CoV-2 protein is the spike surface glycoprotein, membrane glycoprotein M, envelope protein E, and nucleocapsid phosphoprotein, or an antigenic fragment thereof. In embodiments, the spike surface glycoprotein is the S1 or S2 subunit, or an antigenic fragment thereof.

In embodiments, the nucleic acid molecule encoding a protein related to infectivity is mRNA, and the mRNA is optionally in vitro transcribed or synthetic. In embodiments, the mRNA comprises one or more non-canonical nucleotides, optionally selected from pseudouridine and 5-methoxyuridine.

In embodiments, the mRNA encodes SARS-CoV-2 spike surface glycoprotein, membrane glycoprotein M, envelope protein E, and nucleocapsid phosphoprotein, or an antigenic fragment thereof.

In embodiments, the megakaryocyte-derived extracellular vesicles generated from the present methods comprise a nucleic acid encoding a protein having reduced C-C chemokine receptor type 5 (CCR5) and C-X-C chemokine receptor type 4 (CXCR4) activity. In embodiments, the composition comprises megakaryocyte-derived extracellular vesicles, which comprise a nucleic acid molecule encoding a mutant CCR5 or CXCR4.

In embodiments, the megakaryocyte-derived extracellular vesicles generated from the present methods comprise a nucleic acid molecule encoding a gene-editing protein that is capable of reducing C-C chemokine receptor type 5 (CCR5) and C-X-C chemokine receptor type 4 (CXCR4) activity. In embodiments, the nucleic acid molecule encoding a gene-editing protein reduces the chemokine receptor type 5 (CCR5) and C-X-C chemokine receptor type 4 (CXCR4) activity by between about 10% to about 20%. In embodiments, the nucleic acid molecule encoding a gene-editing protein reduces the chemokine receptor type 5 (CCR5) and C-X-C chemokine receptor type 4 (CXCR4) activity by between about 20% to about 30%. In embodiments, the nucleic acid molecule encoding a gene-editing protein reduces the chemokine receptor type 5 (CCR5) and C-X-C chemokine receptor type 4 (CXCR4) activity by between about 30% to about 40%. In embodiments, the nucleic acid molecule encoding a gene-editing protein reduces the chemokine receptor type 5 (CCR5) and C-X-C chemokine receptor type 4 (CXCR4) activity by between about 40% to about 50%. In embodiments, the nucleic acid molecule encoding a gene-editing protein reduces the chemokine receptor type 5 (CCR5) and C-X-C chemokine receptor type 4 (CXCR4) activity by between about 50% to about 60%. In embodiments, the nucleic acid molecule encoding a gene-editing protein reduces the chemokine receptor type 5 (CCR5) and C-X-C chemokine receptor type 4 (CXCR4) activity by between about 60% to about 70%. In embodiments, the nucleic acid molecule encoding a gene-editing protein reduces the chemokine receptor type 5 (CCR5) and C-X-C chemokine receptor type 4 (CXCR4) activity by between about 70% to about 80%.

### Thrombocytopenias/Anemias

In various embodiments, the present invention relates to a method for treating a disease or disorder characterized by abnormal numbers or functionality of a blood cell. Such disease or disorder is, in embodiments, a genetic disease or disorder.

In various embodiments, the present invention relates to a method for treating a disease or disorder of hematopoiesis.

Thrombocytopenias relates to a serum platelet count of less than 150,000/μL. Thrombocytopenias can be stratified into mild, moderate, and severe (corresponding to platelet counts of 75,000-150,000/μL, 50,000-75,000/μL, and less than 50,000/μL, respectively).

In an aspect, the present invention relates to a method for treating a thrombocytopenia, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In an aspect, the present invention relates to a method for treating a thrombocytopenia, comprising administering an effective amount of a composition disclosed herein, wherein the composition comprises megakaryocyte-derived extracellular vesicles, which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In another aspect, the present invention relates to a method for treating a thrombocytopenia, comprising administering an effective amount of a composition comprising a cell which is contacted with a composition disclosed herein in vitro, wherein the composition comprises megakaryocyte-derived extracellular vesicles which comprise a nucleic acid encoding a functional thrombocytopenia-related gene, or a protein product thereof, or a nucleic acid encoding a gene-editing protein capable of creating a functional thrombocytopenia-related gene, or a protein product thereof.

In embodiments, the megakaryocyte-derived extracellular vesicles generated by the presently disclosed methods are used to treat patients having mild thrombocytopenia.

In embodiments, the megakaryocyte-derived extracellular vesicles of the present compositions and methods are used to treat patients having moderate thrombocytopenia. In embodiments, the megakaryocyte-derived extracellular vesicles of the present compositions and methods are used to treat patients having severe thrombocytopenia.

In embodiments, a patient having thrombocytopenia is administered a treatment comprising megakaryocyte-derived extracellular vesicles in combination with a treatment selected from one or more of (a) platelet transfusion and (b) administration of a TPO receptor agonist.

In embodiments, the thrombocytopenia is selected from congenital amegaryocytic thrombocytopenia (CAMT), thrombocytopenia with absent radii, radio ulnar synostosis with congenital thrombocytopenia, X-linked macrothrombocytopenia with thalassemia, GB11b-related thrombocytopenia, X-Linked Thrombocytopenia/Wiskott-Aldrich syndrome, Von Willebrand diseases Type 2B, platelet-type Von Willebrand disease, CYCS-Related thrombocytopenia, immune thrombocytopenia (idiopathic thrombocytopenic purpura), and myeloablation/chemotherapy induced thrombocytopenia.

In embodiments, the thrombocytopenia is CAMT.

In embodiments, the method promotes megakaryopoeisis in the patient.

In embodiments, the method causes an increase in platelet counts in the patient.

In embodiments, the increase in platelet counts is greater than about 100×107 platelets/L, or greater than about 100×108 platelets/L, or greater than about 100×109 platelets/L, or greater than about 110×109 platelets/L, or greater than about 120×109 platelets/L, or greater than about 130×109 platelets/L, or greater than about 140×109 platelets/L, or greater than about 150×109 platelets/L.

In embodiments, the method reduces the likelihood of the patient developing aplastic anemia and/or leukemia.

In embodiments, the method obviates the need for hematopoietic stem cell (HSC) transplantation.

In embodiments, the patient has advanced liver disease. In embodiments, the patient with advanced liver disease has increased concentration of von Willebrand factor as compared to a human without advanced liver disease. In embodiments, the patient with advanced liver disease has decreased concentrations of anticoagulant factors, such as antithrombin and protein C, and/or elevated levels of procoagulant factor VIII.

In embodiments, the patient is an infant.

In embodiments, the method provides a functional thrombopoietin (TPO) receptor in the patient.

In embodiments, the gene is a functional c-Mpl gene or encodes a gene-editing protein that is capable of forming a functional c-Mpl gene.

In embodiments, the disease or disorder is characterized by abnormal (e.g. reduced relative to an undiseased state) blood cell functionality. For instance, in embodiments, the present disease or disorder may not be characterized by a reduction in blood cells numbers but activity (e.g. due to a misfunctional protein).

### Hemoglobinopathies

Hemoglobinopathies are among the most common inherited diseases around the world. In embodiments, the megakaryocyte-derived extracellular vesicles of the present methods and compositions are used to treat a hemoglobinopathy in a patient. In embodiments, the hemoglobinopathy falls into the group of (a) thalassemia syndromes or (b) structural hemoglobin (Hb) variants (abnormal hemoglobins). In embodiments, the thalassemia syndrome is α-thalassemia or β-thalassemia. In embodiments, the structural hemoglobin variant is an Hb variant. In embodiments, the structural hemoglobin variant is HbS, HbE or HbC. In embodiments, the megakaryocyte-derived extracellular vesicles are used to treat one or more of the clinical manifestations of hemoglobinopathies selected from mild hypochromic anemia, moderate hematological disease, and severe, lifelong, transfusion-dependent anemia with multiorgan involvement.

In embodiments, the hemoglobinopathy is sickle cell disease (SCD). Sickle cell disease (SCD) encompasses a group of hematologic disorders caused by a single nucleotide-single gene mutation transposition from a normal adenine to thymine in one or both alleles in the chromosome 11 in the SNP rs334. The transposition of thymine instead of adenine causes the transcription of an abnormal hemoglobin (e.g. HbS) that causes intermittent or permanent episodes of ischemia and/or infarction. Sickle hemoglobin changes the anatomy and elastic properties of normal hemoglobin and make red blood cells contained in sickled hemoglobin more viscous with less capacity to transport and deliver oxygen and nutrients to distal organs and tissues. In embodiments, the present compositions and methods treat heterozygous sickled hemoglobin (a.k.a. sickle cell anemia), in which both alleles are affected with a translocation of thymine (T) instead of adenine (A) in SNP rs334. In embodiments, the present compositions and methods treat heterozygous sickled hemoglobin, in which one allele is affected (A/T).

In another aspect, the present invention relates to a method for treating a hemoglobinopathy, comprising administering an effective amount of a composition disclosed herein, wherein the composition comprises megakaryocyte-derived extracellular vesicles, which comprise cargo. In embodiments, the megakaryocyte-derived extracellular vesicles comprise a lipid bilayer membrane surrounding a lumen and derived from a human pluripotent stem cell, wherein the megakaryocyte-derived extracellular vesicle lumen comprises the cargo. In embodiments, in addition to or as an alternative to the cargo located in the lumen of the megakaryocyte-derived extracellular vesicle, the cargo is associated with the surface of the vesicle. In embodiments, the cargo is selected from one or more of a RNA, DNA, protein, carbohydrate, lipid, biomolecule, and small molecule. In embodiments, the cargo is one or more therapeutic agents.

In an aspect, the present invention relates to a method for treating a hemoglobinopathy, comprising administering an effective amount of a composition disclosed herein, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

In another aspect, the present invention relates to a method for treating a hemoglobinopathy, comprising administering an effective amount of a composition comprising a cell which is contacted with a composition disclosed herein in vitro, wherein the composition comprises megakaryocyte-derived extracellular vesicles which comprise a nucleic acid encoding a functional hemoglobinopathy-related gene, or a protein product thereof, or a nucleic acid encoding a gene-editing protein capable of creating a functional hemoglobinopathy-related gene, or a protein product thereof.

In embodiments, treatment with megakaryocyte-derived extracellular vesicles is combined with one or more of (a) stem cell transplantation; (b) periodic blood transfusions for life, combined with iron chelation; and (c) drugs, including analgesics, antibiotics, ACE inhibitors, and hydroxyurea. In embodiments, treatment with megakaryocyte-derived extracellular vesicles is combined with diagnostic testing. In embodiments, the diagnostic testing is selected from one or more of: (a) iron deficiency test; (b) red blood cell count; (c) DNA test; and (d) hemoglobin test.

In embodiments, the megakaryocyte-derived extracellular vesicles are used to treat a thalassemic hemoglobin synthesis disorder. In embodiments, the megakaryocyte-derived extracellular vesicles are used to treat a patient with abnormal hemoglobins. Sickle cell disease includes all manifestations of abnormal HbS levels, particularly HbS of greater than 50%.

In embodiments, the hemoglobinopathy is sickle cell disease. In embodiments, the hemoglobinopathy is β-thalassemia.

In embodiments, the method reduces or prevents one or more of red cell distortion, hemolytic anemia, microvascular obstruction, and ischemic tissue damage.

In embodiments, the functional hemoglobinopathy-related gene is a gene encoding a portion of hemoglobin. In embodiments, the functional hemoglobinopathy-related gene is a gene encoding one of the globin chains of hemoglobin.

In embodiments, the functional hemoglobinopathy-related gene restores hemoglobin solubility, stability, and/or oxygen affinity to undiseased levels. In embodiments, the functional hemoglobinopathy-related gene restores hemoglobin solubility, stability, and/or oxygen affinity to between about 40% and about 50% of undiseased levels. In embodiments, the functional hemoglobinopathy-related gene restores hemoglobin solubility, stability, and/or oxygen affinity to about 50% and about 60% of undiseased levels. In embodiments, the functional hemoglobinopathy-related gene restores hemoglobin solubility, stability, and/or oxygen affinity to about 60% and about 70% of undiseased levels. In embodiments, the functional hemoglobinopathy-related gene restores hemoglobin solubility, stability, and/or oxygen affinity to about 70% and about 80% of undiseased levels. In embodiments, the functional hemoglobinopathy-related gene restores hemoglobin solubility, stability, and/or oxygen affinity to about 80% and about 90% of undiseased levels. In embodiments, the functional hemoglobinopathy-related gene restores hemoglobin solubility, stability, and/or oxygen affinity to about 90% and about 100% of undiseased levels. In embodiments, the functional hemoglobinopathy-related gene improves hemoglobin solubility, stability, and/or oxygen affinity compared to undiseased levels. In embodiments, the functional hemoglobinopathy-related gene increases hemoglobin solubility, stability, and/or oxygen affinity. In embodiments, the functional hemoglobinopathy-related gene increases hemoglobin solubility, stability, and/or oxygen affinity compared to undiseased levels.

In embodiments, the functional hemoglobinopathy-related gene restores hemoglobin quantity to undiseased levels. In embodiments, the functional hemoglobinopathy-related gene restores hemoglobin quantity to between about 40% and about 50% of undiseased levels. In embodiments, the functional hemoglobinopathy-related gene restores hemoglobin quantity to about 50% and about 60% of undiseased levels. In embodiments, the functional hemoglobinopathy-related gene restores hemoglobin quantity to about 60% and about 70% of undiseased levels. In embodiments, the functional hemoglobinopathy-related gene restores hemoglobin quantity to about 70% and about 80% of undiseased levels. In embodiments, the functional hemoglobinopathy-related gene restores hemoglobin quantity to about 80% and about 90% of undiseased levels. In embodiments, the functional hemoglobinopathy-related gene restores hemoglobin quantity to about 90% and about 100% of undiseased levels. In embodiments, the functional hemoglobinopathy-related gene improves hemoglobin quantity compared to undiseased levels. In embodiments, the functional hemoglobinopathy-related gene increases hemoglobin quantity. In embodiments, the functional hemoglobinopathy-related gene increases hemoglobin quantity compared to undiseased levels

In embodiments, the functional hemoglobinopathy-related gene prevents or reduces RBC sickling.

In embodiments, the functional hemoglobinopathy-related gene prevents or reduces sickle hemoglobin polymerization.

In embodiments, the functional hemoglobinopathy-related gene is beta globin (HBB). In embodiments, the gene encodes a gene-editing protein that is capable of forming a functional beta globin (HBB) gene.

### Pharmaceutical Compositions

Therapeutic treatments comprise the use of one or more routes of administration and of one or more formulations that are designed to achieve a therapeutic effect at an effective dose, while minimizing toxicity to the patient to which treatment is administered.

In embodiments, the effective dose is an amount that substantially avoids cell toxicity in vivo. In various embodiments, the effective dose is an amount that substantially avoids an immune reaction in a human patient. For example, the immune reaction may be an immune response mediated by the innate immune system. Immune response can be monitored using markers known in the art (e.g. cytokines, interferons, TLRs). In embodiments, the effective dose obviates the need for treatment of the human patient with immune suppressants agents used to moderate the residual toxicity.

Upon formulation, solutions may be administered in a manner compatible with the dosage formulation and in such amount as is therapeutically effective, as described herein. The formulations may easily be administered in a variety of dosage forms such as injectable solutions and the like. For parenteral administration in an aqueous solution, for example, the solution generally is suitably buffered and the liquid diluent first rendered isotonic with, for example, sufficient saline or glucose. Such aqueous solutions may be used, for example, for intravenous, intramuscular, subcutaneous and intraperitoneal administration. Preferably, sterile aqueous media are employed as is known to those of skill in the art.

Pharmaceutical preparations may additionally comprise delivery reagents (a.k.a. “transfection reagents”, a.k.a. “vehicles”, a.k.a. “delivery vehicles”) and/or excipients. Pharmaceutically acceptable delivery reagents, excipients, and methods of preparation and use thereof, including methods for preparing and administering pharmaceutical preparations to patients are well known in the art, and are set forth in numerous publications, including, for example, in US Patent Appl. Pub. No. US 2008/0213377, the entirety of which is incorporated herein by reference. In aspects, the present invention relates to a pharmaceutical composition comprising a composition disclosed herein and a pharmaceutically acceptable excipient or carrier.

For example, the megakaryocyte-derived extracellular vesicles generated by the present methods can be in the form of pharmaceutically acceptable salts. Such salts include those listed in, for example, J. Pharma. Sci. 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety. Non-limiting examples of pharmaceutically acceptable salts include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, pamoate, phenylacetate, trifluoroacetate, acrylate, chlorobenzoate, dinitrobenzoate, hydroxybenzoate, methoxybenzoate, methylbenzoate, o-acetoxybenzoate, naphthalene-2-benzoate, isobutyrate, phenylbutyrate, α-hydroxybutyrate, butyne-1,4-dicarboxylate, hexyne-1,4-dicarboxylate, caprate, caprylate, cinnamate, glycollate, heptanoate, hippurate, malate, hydroxymaleate, malonate, mandelate, mesylate, nicotinate, phthalate, teraphthalate, propiolate, propionate, phenylpropionate, sebacate, suberate, p-bromobenzenesulfonate, chlorobenzenesulfonate, ethylsulfonate, 2-hydroxyethylsulfonate, methylsulfonate, naphthalene-1-sulfonate, naphthalene-2-sulfonate, naphthalene-1,5-sulfonate, xylenesulfonate, tartarate salts, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like.

The present pharmaceutical compositions can comprise excipients, including liquids such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In embodiments, the pharmaceutically acceptable excipients are sterile when administered to a patient. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the composition is formulated for one or more of topical, intrathecal, intra-lesional, intra-coronary, intravenous (IV), intra-articular, intramuscular, intra-nasal, and intra-endobronchial administration and administration via intrapancreatic endovascular injection, intra-nucleus pulposus, lumbar puncture, intra-myocardium, transendocardium, intra-fistula tract, intermedullary space, intra-nasal, and intradural space injection.

In embodiments, the composition is formulated for infusion. In embodiments, the composition is formulated for infusion, wherein the composition is delivered to the bloodstream of a patient through a needle in a vein of the patient through a peripheral line, a central line, a tunneled line, an implantable port, and/or a catheter. In embodiments, the patient may also receive supportive medications or treatments, such as hydration, by infusion. In embodiments, the composition is formulated for intravenous infusion. In embodiments, the infusion is continuous infusion, secondary intravenous therapy (IV), and/or IV push. In embodiments, the infusion of the composition may be administered through the use of equipment selected from one or more of an infusion pump, hypodermic needle, drip chamber, peripheral cannula, and pressure bag.

In embodiments, the composition is introduced into or onto the skin, for instance. intraepidermally, intradermally or subcutaneously, in the form of a cosmeceutical (see, e.g., Epstein, H., Clin. Dermatol. 27(5):453-460 (2009)). In embodiments, the composition is in the form of a cream, lotion, ointment, gel, spray, solution and the like. In embodiments, the composition further includes a penetration enhancer such as, but not limited to, surfactants, fatty acids, bile salts, chelating agents, non-chelating non-surfactants, and the like. In embodiments, the composition may also include a fragrance, a colorant, a sunscreen, an antibacterial and/or a moisturizer. In order that the invention disclosed herein may be more efficiently understood, examples are provided below. It should be understood that these examples are for illustrative purposes only and are not to be construed as limiting the invention in any manner.

### Compositions for the Methods of the Invention

Disclosed herein are methods of using a composition comprising megakaryocyte-derived extracellular vesicles.

In one aspect, the present invention relates to methods of making or using a composition comprising: a plurality of megakaryocyte-derived extracellular vesicles comprising a lipid bilayer membrane surrounding a lumen and derived from a human pluripotent stem cell, wherein: the megakaryocyte-derived extracellular vesicle lumen comprises cargo and the lipid bilayer membrane comprises one or more nucleic acid molecules and/or more or more proteins associated with or embedded within. In embodiments, the cargo is one or more therapeutic agents, including therapeutic agents described herein. In embodiments, the cargo comprises one or more megakaryocyte-derived nucleic acid molecules selected from mRNA, tRNA, rRNA, siRNA, microRNA, regulating RNA, and non-coding and coding RNA. In embodiments, in addition to or as an alternative to the cargo located in the lumen of the megakaryocyte-derived extracellular vesicles, the cargo is loaded into the megakaryocyte for packaging into the extracellular vesicles. In embodiments, in addition to or as an alternative to the cargo located in the lumen of the megakaryocyte-derived extracellular vesicles, the cargo is loaded directly into the megakaryocyte-derived extracellular vesicles. In embodiments, the megakaryocyte-derived extracellular vesicles are suitable for loading with cargo associated with the surface of the megakaryocyte-derived extracellular vesicles.

### Biomarker Profile or Fingerprint

In various embodiments, the megakaryocyte-derived extracellular vesicles useful for the present invention are characterized by a unique biomarker profile or fingerprint that distinguishes them from, for instance, naturally-occurring megakaryocyte-derived extracellular vesicles and/or vesicles or extracellular vesicles derived from platelets. In various embodiments, the present megakaryocyte-derived extracellular vesicles are characterized by a such a biomarker profile or fingerprint, which comprises the identity (e.g. the presence or absence) or amount (e.g. substantial presence or substantial absence of a biomarker in a megakaryocyte-derived extracellular vesicle population, or presence on or absence from a majority of megakaryocyte-derived extracellular vesicle in a population, or percentage megakaryocyte-derived extracellular vesicles having a biomarker).

In embodiments, the composition comprises megakaryocyte-derived extracellular vesicles comprising a lipid bilayer membrane surrounding a lumen and derived from a human pluripotent stem cell, wherein the lipid bilayer membrane comprises one or more proteins (a.k.a. biomarkers) associated with or embedded within.

Detailed characteristics of megakaryocyte-derived extracellular vesicles are provided, for example, in international Patent Appl. No. PCT/US2021/031778, the entirety of which is incorporated herein by reference.

In embodiments, the lipid bilayer membrane comprises one or more proteins selected from CD54, CD18, CD43, CD11b, CD62P, CD41, CD61, CD21, CD51, phosphatidylserine (PS), CLEC-2, LAMP-1 (CD107a), CD63, CD42b, CD9, CD31, CD47, CD147, CD32a, and GPVI.

In embodiments, the lipid bilayer membrane comprises one or more proteins selected from CD62P, CD41, and CD61.

In embodiments, greater than about 40%, greater than about 50%, greater than about 60%, greater than about 70%, greater than about 80%, greater than about 90%, greater than about 95%, or greater than about 99% of the megakaryocyte-derived extracellular vesicles comprising a lipid bilayer membrane comprising CD41 also comprise CD61 in the lipid bilayer membrane.

In embodiments, the megakaryocyte-derived extracellular vesicles are characterized by the expression and/or presence of one or more of CD54, CD18, CD43, CD11 b, CD62P, CD41, CD61, CD21, CD51, and CLEC-2. In embodiments, the megakaryocyte-derived extracellular vesicles are characterized by the expression and/or presence of one or more of PS, CD62P, LAMP-1 (CD107a), CD42b, CD9, CD43, CD31, and CD11b. In embodiments, the megakaryocyte-derived extracellular vesicles are characterized by the expression and/or presence of one or more of PS, CD61, CD62P, LAMP-1 (CD107a), CLEC-2, and CD63. In embodiments, the megakaryocyte-derived extracellular vesicles are characterized by the expression and/or presence of one or more of PS, CD62P, CLEC-2, CD9, CD31, CD147, CD32a, and GPVI. In embodiments, the present megakaryocyte-derived extracellular vesicles are characterized by the expression and/or presence of one or more of PS, CD62P, LAMP-1 (CD107a), CLEC-2, CD9, and CD31. In embodiments, the megakaryocyte-derived extracellular vesicles are characterized by the expression and/or presence of one or more of CD62P, CD41, and CD61. In embodiments, the megakaryocyte-derived extracellular vesicles are characterized by a substantial expression and/or presence of one or more of CD54, CD18, CD43, CD11b, CD62P, CD41, CD61, CD21, CD51, and CLEC-2. In embodiments, the megakaryocyte-derived extracellular vesicles are characterized by a substantial expression and/or presence of one or more of CD62P, CD41, and CD61. In embodiments, the present megakaryocyte-derived extracellular vesicles are characterized by not expressing and/or comprising a substantial amount of DRAQ5. In embodiments, the present megakaryocyte-derived extracellular vesicles are characterized by being substantially free of DRAQ5.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD62P.

In embodiments, the megakaryocyte-derived extracellular vesicles are free of, or substantially free of CD62P.

In embodiments, the megakaryocyte-derived extracellular vesicles are characterized by a higher expression, a lower expression and/or presence of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD62P than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, greater than about 50% of the megakaryocyte-derived extracellular vesicles comprise a lipid bilayer membrane comprising CD41. In embodiments, greater than about 40% of the megakaryocyte-derived extracellular vesicles comprise a lipid bilayer membrane comprising CD41. In embodiments, greater than about 60% of the megakaryocyte-derived extracellular vesicles comprise a lipid bilayer membrane comprising CD41. In embodiments, greater than about 70% of the megakaryocyte-derived extracellular vesicles comprise a lipid bilayer membrane comprising CD41. In embodiments, greater than about 80% of the megakaryocyte-derived extracellular vesicles comprise a lipid bilayer membrane comprising CD41. In embodiments, greater than about 90% of the megakaryocyte-derived extracellular vesicles comprise a lipid bilayer membrane comprising CD41. In embodiments, greater than about 95% of the megakaryocyte-derived extracellular vesicles comprise a lipid bilayer membrane comprising CD41. In embodiments, greater than about 99% of the megakaryocyte-derived extracellular vesicles comprise a lipid bilayer membrane comprising CD41.

In embodiments, about 50% or less of the megakaryocyte-derived extracellular vesicles comprise a lipid bilayer membrane comprising CD41. In embodiments, about 40% or less of the megakaryocyte-derived extracellular vesicles comprise a lipid bilayer membrane comprising CD41. In embodiments, about 60% or less of the megakaryocyte-derived extracellular vesicles comprise a lipid bilayer membrane comprising CD41. In embodiments, about 70% or less of the megakaryocyte-derived extracellular vesicles comprise a lipid bilayer membrane comprising CD41. In embodiments, about 80% or less of the megakaryocyte-derived extracellular vesicles comprise a lipid bilayer membrane comprising CD41. In embodiments, about 90% or less of the megakaryocyte-derived extracellular vesicles comprise a lipid bilayer membrane comprising CD41. In embodiments, about 95% or less of the megakaryocyte-derived extracellular vesicles comprise a lipid bilayer membrane comprising CD41. In embodiments, about 99% or less of the megakaryocyte-derived extracellular vesicles comprise a lipid bilayer membrane comprising CD41.

In embodiments, less than about 50% of the megakaryocyte-derived extracellular vesicles comprise a lipid bilayer membrane comprising CD41. In embodiments, less than about 40% of the megakaryocyte-derived extracellular vesicles comprise a lipid bilayer membrane comprising CD41. In embodiments, less than about 30% of the megakaryocyte-derived extracellular vesicles comprise a lipid bilayer membrane comprising CD41. In embodiments, less than about 25% of the megakaryocyte-derived extracellular vesicles comprise a lipid bilayer membrane comprising CD41. In embodiments, less than about 20% of the megakaryocyte-derived extracellular vesicles comprise a lipid bilayer membrane comprising CD41. In embodiments, less than about 15% of the megakaryocyte-derived extracellular vesicles comprise a lipid bilayer membrane comprising CD41. In embodiments, less than about 10% of the megakaryocyte-derived extracellular vesicles comprise a lipid bilayer membrane comprising CD41. In embodiments, less than about 5% of the megakaryocyte-derived extracellular vesicles comprise a lipid bilayer membrane comprising CD41. In embodiments, less than about 1% of the megakaryocyte-derived extracellular vesicles comprise a lipid bilayer membrane comprising CD41.

In embodiments, between about 1% to about 99% of the megakaryocyte-derived extracellular vesicles comprise a lipid bilayer membrane comprising CD41. In embodiments, between about 1% to about 50% of the megakaryocyte-derived extracellular vesicles comprise a lipid bilayer membrane comprising CD41. In embodiments, between about 1% to about 25% of the megakaryocyte-derived extracellular vesicles comprise a lipid bilayer membrane comprising CD41. In embodiments, between about 1% to about 10% of the megakaryocyte-derived extracellular vesicles comprise a lipid bilayer membrane comprising CD41. In embodiments, between about 1% to about 5% of the megakaryocyte-derived extracellular vesicles comprise a lipid bilayer membrane comprising CD41. In embodiments, between about 1% to about 2% of the megakaryocyte-derived extracellular vesicles comprise a lipid bilayer membrane comprising CD41. In embodiments, between about 50% to about 99% of the megakaryocyte-derived extracellular vesicles comprise a lipid bilayer membrane comprising CD41. In embodiments, between about 75% to about 99% of the megakaryocyte-derived extracellular vesicles comprise a lipid bilayer membrane comprising CD41. In embodiments, between about 90% to about 99% of the megakaryocyte-derived extracellular vesicles comprise a lipid bilayer membrane comprising CD41. In embodiments, between about 95% to about 99% of the megakaryocyte-derived extracellular vesicles comprise a lipid bilayer membrane comprising CD41.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD41.

In embodiments, the megakaryocyte-derived extracellular vesicles comprise CD41. In embodiments, the megakaryocyte-derived extracellular vesicles comprise greater than about 40% CD41.

In embodiments, the megakaryocyte-derived extracellular vesicles are characterized by a higher expression and/or presence or CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by a lower expression and/or presence or CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD41 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold to about an 8-fold or about a 2-fold to about a 4-fold greater amount of CD41/CD61 than platelet free plasma (PFP) MkEVs. In embodiments, the megakaryocyte-derived extracellular vesicles have about a 2-fold, about a 3-fold, or about a 4-fold greater amount of CD41/CD61 than platelet free plasma (PFP) MkEVs. In embodiments, the megakaryocyte-derived extracellular vesicles have about a 1.1-fold to about a 2-fold greater amount of CD41/CD61 than platelet derived extracellular vesicles (PLT EVs). In embodiments, the megakaryocyte-derived extracellular vesicles have about a 1.1-fold or about a 1.2-fold greater amount of CD41/CD61 than platelet derived extracellular vesicles (PLT EVs). In embodiments, the megakaryocyte-derived extracellular vesicles have an amount of CD41/CD61 that is substantially the same as platelet derived extracellular vesicles (PLT EVs).

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD61.

In embodiments, the megakaryocyte-derived extracellular vesicles are characterized by a higher expression, a lower expression and/or presence of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD61 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD54.

In embodiments, the megakaryocyte-derived extracellular vesicles are characterized by a higher expression, a lower expression and/or presence of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD54 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD18.

In embodiments, the megakaryocyte-derived extracellular vesicles are characterized by a higher expression, a lower expression and/or presence of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD18 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD43.

In embodiments, the megakaryocyte-derived extracellular vesicles are characterized by a higher expression, a lower expression and/or presence of CD43 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD43 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD11 b.

In embodiments, the megakaryocyte-derived extracellular vesicles are characterized by a higher expression, a lower expression and/or presence of CD11 b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD11 b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD21.

In embodiments, the megakaryocyte-derived extracellular vesicles are characterized by a higher expression, or a lower expression and/or presence of CD21 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD51.

In embodiments, the megakaryocyte-derived extracellular vesicles are characterized by a higher expression, or a lower expression and/or presence of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD51 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CLEC-2.

In embodiments, the megakaryocyte-derived extracellular vesicles are characterized by a higher expression, or a lower expression and/or presence of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CLEC-2 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising LAMP-1 (CD107A).

In embodiments, the megakaryocyte-derived extracellular vesicles are free of, or substantially free of LAMP-1 (CD107A).

In embodiments, the megakaryocyte-derived extracellular vesicles are characterized by presence of LAMP-1 and/or a higher expression or lower expression of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of LAMP-1 (CD107A) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD63.

In embodiments, the megakaryocyte-derived extracellular vesicles are characterized by a higher expression or lower expression and/or presence of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD42b.

In embodiments, the megakaryocyte-derived extracellular vesicles are characterized by a higher expression, or lower expression, and/or presence of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD42b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD9.

In embodiments, the megakaryocyte-derived extracellular vesicles are characterized by a higher expression, or lower expression and/or presence of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD9 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD31.

In embodiments, the megakaryocyte-derived extracellular vesicles are characterized by a higher expression, or lower expression, and/or presence of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD47.

In embodiments, the megakaryocyte-derived extracellular vesicles are characterized by a higher expression, a lower expression and/or presence of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD47 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD147.

In embodiments, the megakaryocyte-derived extracellular vesicles are characterized by a higher expression, or a low expression and/or presence of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD147 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising CD32a.

In embodiments, the megakaryocyte-derived extracellular vesicles are free of, or substantially free of CD32a.

In embodiments, the megakaryocyte-derived extracellular vesicles are characterized by a higher expression, or a lower expression, and/or presence of CD32a than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount or lower amount of CD32a than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, less than about 1%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or greater than about 99% of the megakaryocyte-derived extracellular vesicles comprise a lipid bilayer membrane comprising GVPI.

In embodiments, the megakaryocyte-derived extracellular vesicles are characterized by a higher expression, or lower expression, and/or presence of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount, or lower amount of GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are free of, or substantially free of LAMP-1 (CD107A). In embodiments, the megakaryocyte-derived extracellular vesicles have less LAMP-1 (CD107A) than naturally-occurring megakaryocyte-derived extracellular vesicles and/or vesicles or extracellular vesicles derived from platelets.

In embodiments, less than about 20%, or less than about 15%, or less than about 10%, or less than about 5% of the megakaryocyte-derived extracellular vesicles comprise a lipid bilayer membrane comprising LAMP-1 (CD107A).

In embodiments, the megakaryocyte-derived extracellular vesicles are characterized by having CD62P and being free of, or substantially free of LAMP-1 (CD107A).

In embodiments, the megakaryocyte-derived extracellular vesicles are characterized by a population of megakaryocyte-derived extracellular vesicles wherein less than about 20%, or less than about 15%, or less than about 10%, or less than about 5% of the megakaryocyte-derived extracellular vesicles comprise a lipid bilayer membrane comprising LAMP-1 (CD107A) and greater than about 40%, greater than about 50%, greater than about 60%, greater than about 70%, greater than about 80%, greater than about 90%, greater than about 95%, or greater than about 99% comprises a lipid bilayer membrane comprising CD62P.

In embodiments, less than about 70%, or less than about 60%, or less than about 50%, less than about 40%, less than about 30%, less than about 20%, less than about 15%, less than about 10%, less than about 5%, or less than about 1% of the megakaryocyte-derived extracellular vesicles comprise a lipid bilayer membrane comprising phosphatidylserine (PS).

In embodiments, the megakaryocyte-derived extracellular vesicles are characterized by a lower expression and/or presence of phosphatidylserine (PS) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of phosphatidylserine (PS) than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles.

In embodiments, the megakaryocyte-derived extracellular vesicles are characterized by being free of, or substantially free of phosphatidylserine (PS).

In embodiments, the megakaryocyte-derived extracellular vesicles are characterized by a population of megakaryocyte-derived extracellular vesicles wherein less than about 50%, less than about 40%, less than about 30%, less than about 20%, less than about 15%, less than about 10%, less than about 5%, or less than about 1% of the megakaryocyte-derived extracellular vesicles comprise a lipid bilayer membrane comprising phosphatidylserine (PS), and greater than about 40%, greater than about 50%, greater than about 60%, greater than about 70%, greater than about 80%, greater than about 90%, greater than about 95%, or greater than about 99% of the megakaryocyte-derived extracellular vesicles comprise a lipid bilayer membrane comprising CD47.

In embodiments, the megakaryocyte-derived extracellular vesicles are characterized by a higher expression and/or presence or CD41 than naturally-occurring megakaryocyte-derived extracellular vesicles and/or vesicles or extracellular vesicles derived from platelets. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of CD41 than naturally-occurring megakaryocyte-derived extracellular vesicles and/or vesicles or extracellular vesicles derived from platelets.

In embodiments, the megakaryocyte-derived extracellular vesicles are characterized by a lower expression and/or presence or CD41 than naturally-occurring megakaryocyte-derived extracellular vesicles and/or vesicles or extracellular vesicles derived from platelets. In embodiments, the megakaryocyte-derived extracellular vesicles have about a 2-fold, or about a 10-fold, or about a 50-fold, or about a 100-fold, or about a 300-fold, or about a 500-fold, or about a 1000-fold lower amount of CD41 than naturally-occurring megakaryocyte-derived extracellular vesicles and/or vesicles or extracellular vesicles derived from platelets.

In embodiments, the megakaryocyte-derived extracellular vesicles contain full-length filamin A.

In embodiments, the megakaryocyte-derived extracellular vesicles comprise a lipid bilayer membrane that comprises phosphatidylserine. In embodiments, the megakaryocyte-derived extracellular vesicles are characterized by a population of megakaryocyte-derived extracellular vesicles of which greater than about 40%, greater than about 50%, greater than about 60%, greater than about 70%, greater than about 80%, greater than about 90%, greater than about 95%, or greater than about 99% comprises a lipid bilayer membrane that comprises phosphatidylserine.

In embodiments, the megakaryocyte-derived extracellular vesicles comprise a lipid bilayer membrane positive for Annexin V. For instance, Annexin V, which interacts with phosphatidylserine (PS), can be used as a surrogate for phosphatidylserine expression and/or presence or absence. In embodiments, the megakaryocyte-derived extracellular vesicles are characterized by a population of megakaryocyte-derived extracellular vesicles of which greater than about 40%, greater than about 50%, greater than about 60%, greater than about 70%, greater than about 80%, greater than about 90%, or greater than about 95% are positive for PS.

In embodiments, substantially all of the megakaryocyte-derived extracellular vesicles in the population comprise 2, 3, 4, 5, 6, 7, or 8 of Phosphatidylserine (PS), CD62P, LAMP-1 (CD107a), CD42b, CD9, CD43, CD31, and CD11b. In embodiments, substantially all of the megakaryocyte-derived extracellular vesicles in the population comprise 2, 3, or 4 of PS, CD62P, CD9, and CD11b. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, LAMP-1 (CD107a), CD42b, CD9, CD43, CD31, and CD11b than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, or 6 of Phosphatidylserine (PS), CD61, CD62P, LAMP-1 (CD107a), CLEC-2, and CD63. In embodiments, substantially all of the megakaryocyte-derived extracellular vesicles in the population comprise 2 or 3 of PS, CD61, and CD63. In embodiments, substantially all of the megakaryocyte-derived extracellular vesicles in the population comprise Phosphatidylserine (PS) and CD61. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD61, CD62P, LAMP-1 (CD107a), CLEC-2, and CD63 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, 6, 7, or 8 of Phosphatidylserine (PS), CD62P, CLEC-2, CD9, CD31, CD147, CD32a, and GPVI. In embodiments, substantially all of the megakaryocyte-derived extracellular vesicles in the population comprise 2, 3, or 4 of Phosphatidylserine (PS), CD9, CD31, and CD147. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, CLEC-2, CD9, CD31, CD147, CD32a, and GPVI than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, substantially all of the megakaryocyte-derived extracellular vesicles in the population comprise 2, 3, 4, 5, of 6 of Phosphatidylserine (PS), CD62P, LAMP-1 (CD107a), CLEC-2, CD9, and CD31. In embodiments, substantially all of the megakaryocyte-derived extracellular vesicles in the population comprise 2 or 3 of Phosphatidylserine (PS), CD62P, and CD9. In embodiments, substantially all of the megakaryocyte-derived extracellular vesicles in the population comprise PS and CD9. In embodiments, the megakaryocyte-derived extracellular vesicles have about a 2-fold, or about a 10-fold, or about a 50-fold, or about a 100-fold, or about a 300-fold, or about a 500-fold, or about a 1000-fold greater amount of one or more of Phosphatidylserine (PS), CD62P, LAMP-1 (CD107a), CLEC-2, CD9, and CD31 than naturally-occurring megakaryocyte-derived extracellular vesicles, vesicles or extracellular vesicles derived from platelets such as platelet derived extracellular vesicles (PLT EVs), and/or platelet-free plasma (PPF) megakaryocyte-derived extracellular vesicles. In embodiments, the megakaryocyte-derived extracellular vesicles are characterized by not expressing a substantial amount of DRAQ5. In embodiments, the megakaryocyte-derived extracellular vesicles are characterized by being substantially free of DRAQ5.

In embodiments, the megakaryocyte-derived extracellular vesicles and/or plurality of megakaryocyte-derived extracellular vesicles and/or population of megakaryocyte-derived extracellular vesicles comprise a lipid bilayer membrane, wherein
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD54, and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD18 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD43 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD11 b and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD62P and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD41 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD21 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD51 and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD61 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD147 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD31 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD47 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD32a and/or
greater than about 40%, or greater than about 50%, or greater than about 60%, or greater than about 70%, or greater than about 80%, or greater than about 90%, greater than about 95%, or greater than about 99% of the megakaryocyte-derived extracellular vesicles comprise a lipid bilayer membrane comprising CD9 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CLEC-2 and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising LAMP-1 (CD107a) and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD24b and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising GVPI and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising CD63, and/or
less than about 50%, or less than about 40%, or less than about 30%, or less than about 20%, or less than about 10%, or less than about 5% or less than about 1% of the megakaryocyte-derived extracellular vesicles comprise a lipid bilayer membrane comprising phosphatidylserine (PS). In embodiments, greater than about 40%, greater than about 50%, or greater than about 60%, or greater than about 70%, or greater than about 80%, or greater than about 90%, greater than about 95%, or greater than about 99% of the megakaryocyte-derived extracellular vesicles and/or plurality of megakaryocyte-derived extracellular vesicles and/or population of megakaryocyte-derived extracellular vesicles comprise a lipid bilayer membrane comprising CD41.

### Size Profile or Fingerprint

In various embodiments, the present megakaryocyte-derived extracellular vesicles are characterized by a unique size (e.g. vesicle diameter) profile or fingerprint that distinguishes them from, for instance, naturally-occurring megakaryocyte-derived extracellular vesicles and/or vesicles or extracellular vesicles derived from platelets. In various embodiments, the present megakaryocyte-derived extracellular vesicles are characterized by a such a size profile or fingerprint, which favors larger particles, e.g. as compared to naturally-occurring megakaryocyte-derived extracellular vesicles and/or vesicles or extracellular vesicles derived from platelets, that are desirable for, e.g., their higher carrying capacity.

In various embodiments, the present megakaryocyte-derived extracellular vesicles are characterized by a bias for particles of about 30 nm to about 100 nm.

In various embodiments, the present megakaryocyte-derived extracellular vesicles are characterized by a bias for particles of about 30 nm to about 400 nm.

In various embodiments, the present megakaryocyte-derived extracellular vesicles are characterized by a bias for particles of about 100 nm to about 200 nm.

In various embodiments, the present megakaryocyte-derived extracellular vesicles are characterized by a bias for particles of about 100 nm to about 300 nm.

In various embodiments, the present megakaryocyte-derived extracellular vesicles are characterized by a bias for particles of about 100 nm to about 500 nm.

In various embodiments, the present megakaryocyte-derived extracellular vesicles are characterized by a bias for particles of about 100 nm to about 600 nm.

In various embodiments, the present megakaryocyte-derived extracellular vesicles are characterized by a bias for particles of about 200 nm in diameter, on average.

In various embodiments, the present megakaryocyte-derived extracellular vesicles are characterized by a bias for particles of about 250 nm in diameter, on average.

In embodiments, the megakaryocyte-derived extracellular vesicles are substantially of a diameter of less than about 100 nm. In embodiments, the megakaryocyte-derived extracellular vesicles are substantially of a diameter in the range between about 30 nm to about 300 nm. In embodiments, the megakaryocyte-derived extracellular vesicles are substantially of a diameter in the range between about 30 nm to about 400 nm. In embodiments, the megakaryocyte-derived extracellular vesicles are substantially of a diameter in the range between about 100 nm to about 300 nm. In embodiments, the megakaryocyte-derived extracellular vesicles are substantially of a diameter in the range between about 200 nm to about 300 nm. In embodiments, the megakaryocyte-derived extracellular vesicles are substantially of a diameter in the range between about 300 nm to about 400 nm. In embodiments, the megakaryocyte-derived extracellular vesicles are substantially of a diameter in the range between about 400 nm to about 500 nm. In embodiments, the megakaryocyte-derived extracellular vesicles are substantially of a diameter in the range between about 500 nm to about 600 nm. In embodiments, the megakaryocyte-derived extracellular vesicles are substantially of a diameter in the range between about 600 nm to about 700 nm. In embodiments, the megakaryocyte-derived extracellular vesicles are substantially of a diameter in the range between about 700 nm to about 800 nm. In embodiments, the megakaryocyte-derived extracellular vesicles are substantially of a diameter in the range between about 800 nm to about 900 nm. In embodiments, the megakaryocyte-derived extracellular vesicles are substantially of a diameter in the range between about 900 nm to about 1000 nm. In embodiments, the megakaryocyte-derived extracellular vesicles are substantially of a diameter in the range between about 500 nm to about 1000 nm. In embodiments, the megakaryocyte-derived extracellular vesicles are substantially of a diameter in the range between about 600 nm to about 1000 nm. In embodiments, the megakaryocyte-derived extracellular vesicles are substantially of a diameter in the range between about 100 nm to about 500 nm. In embodiments, the megakaryocyte-derived extracellular vesicles are substantially of a diameter in the range between about 100 nm to about 600 nm. In embodiments, the megakaryocyte-derived extracellular vesicles are substantially of a diameter in the range between about 150 nm to about 500 nm. In embodiments, the megakaryocyte-derived extracellular vesicles are substantially of a diameter in the range between about 100 nm to about 200 nm. In embodiments, the megakaryocyte-derived extracellular vesicles are substantially of a diameter in the range between about 100 nm to about 200 nm. In embodiments, the megakaryocyte-derived extracellular vesicles are substantially of a diameter in the range between about 200 nm to about 600 nm. In embodiments, the megakaryocyte-derived extracellular vesicles are substantially of a diameter in the range between about 30 nm to 100 nm, or between about 30 nm to 400 nm, or between about 100 nm to about 200 nm, or between about 100 nm to about 500 nm, or between about 200 nm to about 350 nm, or between about 400 nm to about 600 nm.

In embodiments, about 90% or more, or about 95% or more, or about 97% or more, or about 99% or more of the megakaryocyte-derived extracellular vesicles are of a diameter of between about 30 to 100 nm.

In embodiments, about 90% or more, or about 95% or more, or about 97% or more, or about 99% or more of the megakaryocyte-derived extracellular vesicles are of a diameter of between about 30 to 400 nm.

In embodiments, about 90% or more, or about 95% or more, or about 97% or more, or about 99% or more of the megakaryocyte-derived extracellular vesicles are of a diameter of between about 100 nm to about 200 nm.

In embodiments, about 90% or more, or about 95% or more, or about 97% or more, or about 99% or more of the megakaryocyte-derived extracellular vesicles are of a diameter of between about 100 nm to about 300 nm.

In embodiments, about 90% or more, or about 95% or more, or about 97% or more, or about 99% or more of the megakaryocyte-derived extracellular vesicles are of a diameter of between about 200 nm to about 350 nm.

In embodiments, about 90% or more, or about 95% or more, or about 97% or more, or about 99% or more of the megakaryocyte-derived extracellular vesicles are of a diameter of between about 100 nm to about 600 nm.

In embodiments, about 90% or more, or about 95% or more, or about 97% or more, or about 99% or more of the megakaryocyte-derived extracellular vesicles are of a diameter of between about 400 nm to about 600 nm.

In embodiments, about 90% or more, or about 95% or more, or about 97% or more, or about 99% or more of the megakaryocyte-derived extracellular vesicles are of a diameter of between about 200 nm to about 600 nm.

In embodiments, about 90% or more, or about 95% or more, or about 97% or more, or about 99% or more of the megakaryocyte-derived extracellular vesicles are of a diameter of between about 30 to about 100 nm and/or about 30 to about 400 nm and/or about 100 nm to about 200 nm and/or about 100 nm to about 300 nm and/or between about 200 nm to about 350 nm and/or between about 400 nm to about 600 nm.

In embodiments, the present compositions comprise various subpopulations of vesicles of different diameter. For example, in embodiments, present compositions comprise one or more of (e.g. one, or two, or three, or four of): a subpopulation of about 50 nm in diameter, a subpopulation of about 150 nm in diameter, a subpopulation of about 200 nm in diameter, a subpopulation of about 250 nm in diameter, a subpopulation of about 300 nm in diameter, a subpopulation of about 400 nm in diameter, a subpopulation of about 500 nm in diameter and a subpopulation of about 600 nm in diameter. In embodiments, present compositions comprise one or more of (e.g. one, or two, or three, or four of): a subpopulation of about 45 nm in diameter, a subpopulation of about 135 nm in diameter, a subpopulation of about 285 nm in diameter, and a subpopulation of about 525 nm in diameter.

In embodiments, about 90% or more, or about 95% or more, or about 97% or more, or about 99% or more of the megakaryocyte-derived extracellular vesicles are of about 50 nm in diameter and/or about 150 nm in diameter and/or about 300 nm in diameter and/or about 500 nm in diameter.

In embodiments, the population of megakaryocyte-derived extracellular vesicles exhibits the following characteristics:
a) about 80% or more, about 85% or more, about 90% or more, about 95% or more, about 97% or more, or about 99% or more of the megakaryocyte-derived extracellular vesicles in the population are substantially free of nuclei;
b) about 90% or more, or about 95% or more, or about 97% or more, or about 99% or more of the megakaryocyte-derived extracellular vesicles are of a diameter of between about 100 nm to about 600 nm.;
c) about 40% or more, about 50% or more, about 60% or more, about 70% or more, about 80% or more, or about 90% or more of the megakaryocyte-derived extracellular vesicles in the population comprise CD41; and
d) the population comprises about 1×10⁷ or more, about 1.5×10⁷ or more, about 5×10⁷ or more, about 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

In embodiments, the population of megakaryocyte-derived extracellular vesicles exhibits the following characteristics:
a) about 80% or more, about 85% or more, about 90% or more, about 95% or more, about 97% or more, or about 99% or more of the megakaryocyte-derived extracellular vesicles in the population are substantially free of nuclei;
b) about 90% or more, or about 95% or more, or about 97% or more, or about 99% or more of the megakaryocyte-derived extracellular vesicles are of a diameter of between about 100 nm to about 600 nm.;
c) about 40% or more, about 50% or more, about 60% or more, about 70% or more, about 80% or more, or about 90% or more of the megakaryocyte-derived extracellular vesicles in the population comprise CD61; and
d) the population comprises about 1×10⁷ or more, about 1.5×10⁷ or more, about 5×10⁷ or more, about 1×10⁸ or more, about 1.5×10⁸ or more, about 5×10⁸ or more, about 1×10⁹ or more, about 5×10⁹ or more, about 1×10¹⁰ or more, or about 1×10¹⁰ or more megakaryocyte-derived extracellular vesicles.

Any method for determining the amount of nuclei in the population of megakaryocyte-derived extracellular vesicles is contemplated by the present disclosure. Non-limiting examples of methods include staining the megakaryocyte-derived extracellular vesicles with a nuclear stain such as DRAQ5, wherein a lack of staining indicates that the megakaryocyte-derived extracellular vesicles are substantially free of nuclei.

### Sources and Characterization of Megakaryocyte-Derived Extracellular Vesicles

Megakaryocytes are large, polyploid cells derived from hematopoietic stem and progenitor cells, contained within the CD34+-cell compartment. In embodiments, the megakaryocyte is characterized by the expression and/or presence of one or more of CD41, CD62P, GPVI, CLEC-2, CD42b and CD61. In embodiments, the megakaryocyte is one or more of CD42b+, CD61+, and DNA+. One morphological characteristic of mature megakaryocytes is the development of a large, multi-lobed nucleus. Mature megakaryocytes can stop proliferating, but continue to increase their DNA content through endomitosis, with a parallel increase in cell size.

In embodiments, in addition to extracellular vesicles, megakaryocytes can shed pre- and proplatelets and platelet-like particles. These shed moieties can mature into platelets. In embodiments, the pre- and proplatelets and platelet-like particles are all different products, which can be differentiated by size, morphology, biomarker expression and/or presence, and function.

In embodiments, megakaryocytes are derived from human pluripotent stem cells. In embodiments, the human pluripotent stem cells are selected from induced pluripotent stem cells (iPSCs), hematopoietic stem cells (HSCs), and embryonic stem cells (ESCs).

In embodiments, megakaryocytes are derived from pluripotent hematopoietic stem cell (HSC) precursors. HSCs are produced primarily by the liver, kidney, spleen, and bone marrow and are capable of producing a variety of blood cells depending on the signals they receive.

Thrombopoietin (TPO) is a primary signal for inducing an HSC to differentiate into a megakaryocyte. Other molecular signals for inducing megakaryocyte differentiation include granulocyte-macrophage colony-stimulating factor (GM-CSF), Interleukin-3 (IL-3), IL-6, IL-11, SCF, fms-like tyrosine kinase 3 ligand (FLT3L), interleukin 9 (IL-9), and the like. Production details are also described elsewhere herein.

In embodiments, the composition comprises substantially purified megakaryocyte-derived extracellular vesicles derived from a human pluripotent stem cell.

In embodiments, the human pluripotent stem cell is a primary CD34+ hematopoietic stem cell. In embodiments, the primary CD34+ hematopoietic stem cell is sourced from peripheral blood or cord blood. In embodiments, the peripheral blood is granulocyte colony-stimulating factor-mobilized adult peripheral blood (mPB). In embodiments, the human pluripotent stem cell is an HSC produced by the liver, kidney, spleen, or bone marrow. In embodiments, the HSC is produced by the liver. In embodiments, the HSC is produced by the kidney. In embodiments, the HSC is produced by the spleen. In embodiments, the HSC is produced by the bone marrow. In embodiments, the HSC is induced to differentiate into a megakaryocyte by receiving a molecular signal selected from one or more of TPO, GM-CSF, IL-3, IL-6, IL-11, SCF, Flt3L, IL-9, and the like. In embodiments, the molecular signal is TPO. In embodiments, the molecular signal is GM-CSF. In embodiments, the molecular signal is IL-3. In embodiments, the molecular signal is IL-6. In embodiments, the molecular signal is IL-11. In embodiments, the molecular signal is IL-6. In embodiments, the molecular signal is SCF. In embodiments, the molecular signal is IL-6. In embodiments, the molecular signal is Flt3L. In embodiments, the molecular signal is IL-6. In embodiments, the molecular signal is IL-9.

In embodiments, the molecular signal is a chemokine.

In embodiments, the molecular signal promotes cell fate decision toward megakaryopoiesis.

In embodiments, the molecular signal is devoid of erythropoietin (EPO).

In embodiments, the human pluripotent stem cell is an embryonic stem cell (ESC). ESCs have the capacity to form cells from all three germ layers of the body, regardless of the method by which the ESCs are derived. ESCs are functionally stem cells that can have one or more of the following characteristics: (a) be capable of inducing teratomas when transplanted in immunodeficient mice; (b) be capable of differentiating to cell types of all three germ layers (i.e. ectodermal, mesodermal, and endodermal cell types); and (c) express one or more markers of embryonic stem cells (e.g., Oct 4, alkaline phosphatase. SSEA-3 surface antigen, SSEA-4 surface antigen, SSEA-5 surface antigen, Nanog, TRA-I-60, TRA-1-81, SOX2, REX1, and the like).

In embodiments, the human pluripotent stem cell is an induced pluripotent stem cell (iPCs). Mature differentiated cells can be reprogrammed and dedifferentiated into embryonic-like cells, with embryonic stem cell-like properties. iPSCs can be generated using fetal, postnatal, newborn, juvenile, or adult somatic cells. Fibroblast cells can be reversed into pluripotency via, for example, retroviral transduction of certain transcription factors, resulting in iPSs. In embodiments, iPSs are generated from various tissues, including fibroblasts, keratinocytes, melanocyte blood cells, bone marrow cells, adipose cells, and tissue-resident progenitor cells. In embodiments, iPSCs are generated via one or more reprogramming or Yamanaka factors, e.g. Oct3/4, Sox2, Klf4, and c-Myc. In certain embodiments, at least two, three, or four reprogramming factors are expressed in a somatic cell to reprogram the somatic cell.

Once a pluripotent cell has completed differentiation and become a mature megakaryocyte, it begins the process of producing platelets, which do not contain a nucleus and may be about 1-3 um in diameter. Megakaryocytes also produce extracellular vesicles.

In embodiments, the present megakaryocytes are induced to favor production of megakaryocyte-derived extracellular vesicles over platelets. That is, in embodiments, the present megakaryocytes produce substantially more megakaryocyte-derived extracellular vesicles than platelets. In embodiments, the present compositions are substantially free of platelets. In embodiments, the present compositions contain less than about 10%, or less than about 7%, or less than about 5%, or less than about 3%, or less than about 2%, or less than about 1% platelets.

In embodiments, the present compositions are substantially free of extracellular vesicles derived from platelets. In embodiments, the present compositions contain less than about 10%, or less than about 7%, or less than about 5%, or less than about 3%, or less than about 2%, or less than about 1% of extracellular vesicles derived from platelets.

In embodiments, the megakaryocyte-derived extracellular vesicles of the disclosure are substantially free of organelles. Non-limiting examples of contaminating organelles include, but are not limited to, mitochondria, and nuclei. In embodiments, the megakaryocyte-derived extracellular vesicles of the disclosure are substantially free of mitochondria. In embodiments, the preparation comprising the megakaryocyte-derived extracellular vesicles of the disclosure is substantially free of exosomes. In embodiments, megakaryocyte-derived extracellular vesicles of the disclosure comprise organelles.

In embodiments, the megakaryocyte-derived extracellular vesicles of the disclosure are substantially free of nuclei. In embodiments, about 80% to about 100%, about 85% to about 100%, about 90% to about 100%, or about 95% to about 100% of the megakaryocyte-derived extracellular vesicles in the population are substantially free of nuclei. In embodiments, greater than about 80%, greater than about 85%, greater than about 90%, greater than about 95%, greater than about 99%, or about 100% of the megakaryocyte-derived extracellular vesicles in the population are substantially free of nuclei.

### Cargo of Megakaryocyte-Derived Extracellular Vesicles

Megakaryocyte-derived extracellular vesicles may contain diverse cargo such as mRNAs, microRNAs, and cytokines. Megakaryocyte-derived extracellular vesicles are able to transfer their cargo to alter the function of target cells. They exert their influence on the target cells through surface receptor signaling, plasma membrane fusion, and internalization. By loading megakaryocytes or megakaryocyte-derived extracellular vesicles with biologic or therapeutic cargo, megakaryocyte-derived extracellular vesicles can be further used as delivery vehicles to achieve a targeted therapeutic effect. Until now, small RNAs (siRNA and miRNA), small linear DNA, and plasmid DNA have all been successfully loaded into megakaryocyte-derived extracellular vesicles for a variety of delivery applications. Megakaryocyte-derived extracellular vesicles targeting is defined by their complement of surface proteins and can be further engineered to express or remove specific biomarkers of interest to refine biodistribution and cell-cell recognition. For instance, the present megakaryocyte-derived extracellular vesicles, with their unique biomarker profiles, are particularly suited for delivery of payloads, e.g. therapies.

In embodiments, the megakaryocyte-derived extracellular vesicles are suitable for loading with cargo into the lumen. In embodiments, the cargo is selected from one or more of a RNA, DNA, protein, carbohydrate, lipid, biomolecule, and small molecule. In embodiments, the cargo is a biologically produced component. In embodiments, the cargo is a synthetically produced component. In embodiments, the cargo is pre-loaded into megakaryocyte-derived extracellular vesicles. In embodiments, a biological component is overexpressed in megakaryocytes so that generated megakaryocyte-derived extracellular vesicles comprise the biological component. In embodiments, the cargo is post-loaded into megakaryocyte-derived extracellular vesicles. In embodiments, purified megakaryocyte-derived extracellular vesicles are mixed with cargo to generate cargo-loaded megakaryocyte-derived extracellular vesicles. In embodiments, the cargo is hydrophobic. In embodiments, the cargo is hydrophilic. In embodiments, the cargo is integrated into the lipid bilayer of the megakaryocyte-derived extracellular vesicles. In embodiments, the cargo is located in the lumen of the megakaryocyte-derived extracellular vesicles.

In embodiments, in addition to or as an alternative to the cargo located in the lumen of the megakaryocyte-derived extracellular vesicles, the cargo is associated with the megakaryocyte-derived extracellular vesicles. In embodiments, the cargo is associated with the surface and/or the exterior of the megakaryocyte-derived extracellular vesicles. Non-limiting examples of cargo associated with the megakaryocyte-derived extracellular vesicles includes cargo that is covalently conjugated to the surface of the vesicle or cargo that is associated with the surface via electrostatic interactions. As would be understood by one of ordinary skill in the art, cargo associated with the megakaryocyte-derived extracellular vesicles can still be transported even when not loaded into the lumen of the vesicle.

In embodiments, the loading ratio of a nucleic acid (i.e. copies of nucleic acid per vesicle) into megakaryocyte-derived extracellular vesicles of the disclosure ranges from about 1 to about 1000, about 1 to about 500, about 1 to about 100, about 10 to about 1000, about 100 to about 1000, about 500 to about 1000, about 100 to about 500,000, about 1000 to about 300,000, about 100,000 to about 300,000, about 1000, to about 10,000, or about 1000 to about 5000. In embodiments, the nucleic acid is DNA. In embodiments, the nucleic acid is plasmid DNA.

In embodiments, the loading efficiency for loading cargo, such as a nucleic acid, into megakaryocyte-derived extracellular vesicles of the disclosure ranges from about 1% to about 99%, about 10% to about 90%, about 30% to about 70%, about 40% to about 60%, about 40% to about 50%, or about 50% to about 60%. In embodiments, the cargo is a nucleic acid. In embodiments, the nucleic acid is DNA. In embodiments, the nucleic acid is plasmid DNA. In embodiments, loading efficiency is calculated using the following equation:

$$Loading efficiency ( \% ) = cargo + MV \# / Total MV \#$$

In embodiments, the surface of megakaryocyte-derived extracellular vesicles is modified to impact biodistribution and targeting capabilities of megakaryocyte-derived extracellular vesicles. In embodiments, surface ligands are added to megakaryocyte-derived extracellular vesicles through genetic engineering. In embodiments, the megakaryocyte-derived extracellular vesicles are generated that express fusion proteins in their lipid bilayers. In embodiments, the endogenous proteins in megakaryocyte-derived extracellular vesicle lipid bilayers are fused with targeting ligands through cell engineering.

In embodiments, the cargo is one or more therapeutic agents. In embodiments, the therapeutic agent is a nucleic acid therapeutic agent. In embodiments, the nucleic acid therapeutic agent encodes a functional protein.

In embodiments, the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids. In embodiments, the nucleic acid therapeutic agent is selected from one or more of mRNA, miRNA, siRNA, and snoRNA.

In embodiments, the nucleic acid therapeutic agent encodes a wild type gene, which is defective in the patient. In embodiments, the nucleic acid therapeutic agent is mRNA, and optionally: is in vitro transcribed or synthetic and/or comprises one or more non-canonical nucleotides, optionally selected from pseudouridine and 5-methoxyuridine.

In embodiments, the one or more non-canonical nucleotides are selected from 2-thiouridine, 5-azauridine, pseudouridine, 4-thiouridine, 5-methyluridine, 5-methylpseudouridine, 5-aminouridine, 5-aminopseudouridine, 5-hydroxyuridine, 5-hydroxypseudouridine, 5-methoxyuridine, 5-methoxypseudouridine, 5-ethoxyuridine, 5-ethoxypseudouridine, 5-hydroxymethyluridine, 5-hydroxymethylpseudouridine, 5-carboxyuridine, 5-carboxypseudouridine, 5-formyluridine, 5-formylpseudouridine, 5-methyl-5-azauridine, 5-amino-5-azauridine, 5-hydroxy-5-azauridine, 5-methylpseudouridine, 5-aminopseudouridine, 5-hydroxypseudouridine, 4-thio-5-azauridine, 4-thiopseudouridine, 4-thio-5-methyluridine, 4-thio-5-aminouridine, 4-thio-5-hydroxyuridine, 4-thio-5-methyl-5-azauridine, 4-thio-5-amino-5-azauridine, 4-thio-5-hydroxy-5-azauridine, 4-thio-5-methylpseudouridine, 4-thio-5-aminopseudouridine, 4-thio-5-hydroxypseudouridine, 2-thiocytidine, 5-azacytidine, pseudoisocytidine, N4-methylcytidine, N4-aminocytidine, N4-hydroxycytidine, 5-methylcytidine, 5-aminocytidine, 5-hydroxycytidine, 5-methoxycytidine, 5-ethoxycytidine, 5-hydroxymethylcytidine, 5-carboxycytidine, 5-formylcytydine, 5-methyl-5-azacytidine, 5-amino-5-azacytidine, 5-hydroxy-5-azacytidine, 5-methylpseudoisocytidine, 5-aminopseudoisocytidine, 5-hydroxypseudoisocytidine, N4-methyl-5-azacytidine, N4-methylpseudoisocytidine, 2-thio-5-azacytidine, 2-thiopseudoisocytidine, 2-thio-N4-methylcytidine, 2-thio-N4-aminocytidine, 2-thio-N4-hydroxycytidine, 2-thio-5-methylcytidine, 2-thio-5-aminocytidine, 2-thio-5-hydroxycytidine, 2-thio-5-methyl-5-azacytidine, 2-thio-5-amino-5-azacytidine, 2-thio-5-hydroxy-5-azacytidine, 2-thio-5-methylpseudoisocytidine, 2-thio-5-aminopseudoisocytidine, 2-thio-5-hydroxypseudoisocytidine, 2-thio-N4-methyl-5-azacytidine, 2-thio-N4-methylpseudoisocytidine, N4-methyl-5-methylcytidine, N4-methyl-5-aminocytidine, N4-methyl-5-hydroxycytidine, N4-methyl-5-methyl-5-azacytidine, N4-methyl-5-amino-5-azacytidine, N4-methyl-5-hydroxy-5-azacytidine, N4-methyl-5-methylpseudoisocytidine, N4-methyl-5-aminopseudoisocytidine, N4-methyl-5-hydroxypseudoisocytidine, N4-amino-5-azacytidine, N4-aminopseudoisocytidine, N4-amino-5-methylcytidine, N4-amino-5-aminocytidine, N4-amino-5-hydroxycytidine, N4-amino-5-methyl-5-azacytidine, N4-amino-5-amino-5-azacytidine, N4-amino-5-hydroxy-5-azacytidine, N4-amino-5-methylpseudoisocytidine, N4-amino-5-aminopseudoisocytidine, N4-amino-5-hydroxypseudoisocytidine, N4-hydroxy-5-azacytidine, N4-hydroxypseudoisocytidine, N4-hydroxy-5-methylcytidine, N4-hydroxy-5-aminocytidine, N4-hydroxy-5-hydroxycytidine, N4-hydroxy-5-methyl-5-azacytidine, N4-hydroxy-5-amino-5-azacytidine, N4-hydroxy-5-hydroxy-5-azacytidine, N4-hydroxy-5-methylpseudoisocytidine, N4-hydroxy-5-aminopseudoisocytidine, N4-hydroxy-5-hydroxypseudoisocytidine, 2-thio-N4-methyl-5-methylcytidine, 2-thio-N4-methyl-5-aminocytidine, 2-thio-N4-methyl-5-hydroxycytidine, 2-thio-N4-methyl-5-methyl-5-azacytidine, 2-thio-N4-methyl-5-amino-5-azacytidine, 2-thio-N4-methyl-5-hydroxy-5-azacytidine, 2-thio-N4-methyl-5-methylpseudoisocytidine, 2-thio-N4-methyl-5-aminopseudoisocytidine, 2-thio-N4-methyl-5-hydroxypseudoisocytidine, 2-thio-N4-amino-5-azacytidine, 2-thio-N4-aminopseudoisocytidine, 2-thio-N4-amino-5-methylcytidine, 2-thio-N4-amino-5-aminocytidine, 2-thio-N4-amino-5-hydroxycytidine, 2-thio-N4-amino-5-methyl-5-azacytidine, 2-thio-N4-amino-5-amino-5-azacytidine, 2-thio-N4-amino-5-hydroxy-5-azacytidine, 2-thio-N4-amino-5-methylpseudoisocytidine, 2-thio-N4-amino-5-aminopseudoisocytidine, 2-thio-N4-amino-5-hydroxypseudoisocytidine, 2-thio-N4-hydroxy-5-azacytidine, 2-thio-N4-hydroxypseudoisocytidine, 2-thio-N4-hydroxy-5-methylcytidine, N4-hydroxy-5-aminocytidine, 2-thio-N4-hydroxy-5-hydroxycytidine, 2-thio-N4-hydroxy-5-methyl-5-azacytidine, 2-thio-N4-hydroxy-5-amino-5-azacytidine, 2-thio-N4-hydroxy-5-hydroxy-5-azacytidine, 2-thio-N4-hydroxy-5-methylpseudoisocytidine, 2-thio-N4-hydroxy-5-aminopseudoisocytidine, 2-thio-N4-hydroxy-5-hydroxypseudoisocytidine, N6-methyladenosine, N6-aminoadenosine, N6-hydroxyadenosine, 7-deazaadenosine, 8-azaadenosine, N6-methyl-7-deazaadenosine, N6-methyl-8-azaadenosine, 7-deaza-8-azaadenosine, N6-methyl-7-deaza-8-azaadenosine, N6-amino-7-deazaadenosine, N6-amino-8-azaadenosine, N6-amino-7-deaza-8-azaadenosine, N6-hydroxyadenosine, N6-hydroxy-7-deazaadenosine, N6-hydroxy-8-azaadenosine, N6-hydroxy-7-deaza-8-azaadenosine, 6-thioguanosine, 7-deazaguanosine, 8-azaguanosine, 6-thio-7-deazaguanosine, 6-thio-8-azaguanosine, 7-deaza-8-azaguanosine, and 6-thio-7-deaza-8-azaguanosine.

In embodiments, the present methods comprise gene-editing and/or gene correction and/or provide methods of making vesicles for gene-editing and/or gene correction. In embodiments, the present methods encompass synthetic RNA-based gene-editing and/or gene correction and/or provide methods of making vesicles for gene-editing and/or gene correction, e.g. with RNA comprising non-canonical nucleotides, e.g. RNA encoding one or more of a nuclease, a transcription activator-like effector nuclease (TALEN), a zinc-finger nuclease, a meganuclease, a nickase, a clustered regularly interspaced short palindromic repeat (CRISPR)-associated protein a DNA-repair protein, a DNA-modification protein, a base-modification protein, a DNA methyltransferase, a protein that causes DNA demethylation, an enzyme for which DNA is a substrate or a natural or engineered variant, family-member, orthologue, fragment or fusion construct thereof. In embodiments, the efficiency of the gene-editing and/or gene correction is high, for example, higher than DNA-based gene editing and/or gene correction. In embodiments, the present methods of gene-editing and/or gene correction and/or methods of making vesicles for gene-editing and/or gene correction are efficient enough for in vivo application. In embodiments, the present methods of gene-editing and/or gene correction and/or methods of making vesicles for gene-editing and/or gene correction are efficient enough to not require cellular selection (e.g. selection of cells that have been edited). In embodiments, the efficiency of gene-editing of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100%. In embodiments, the efficiency of gene-correction of the present methods is about 1%, or about 2%, or about 3%, or about 4%, or about 5%, or about 6%, or about 7%, or about 8%, or about 9%, or about 10%, or about 20%, or about 30%, or about 40%, or about 50%, or about 60%, or about 70%, or about 80%, or about 90%, or about 100% In embodiments, the present methods comprise high-efficiency gene-editing proteins comprising engineered nuclease cleavage or DNA-modification domains and/or provide methods of making vesicles for gene correction and/or gene-editing proteins.

In embodiments, the methods comprise high-fidelity gene-editing proteins comprising engineered nuclease cleavage or DNA-modification domains and/or provide methods of making vesicles for gene correction and/or gene-editing proteins. In embodiments, the high-efficiency gene-editing proteins comprising engineered DNA-binding domains. In embodiments, the high-fidelity gene-editing proteins comprising engineered DNA-binding domains. In embodiments, the methods comprise gene-editing proteins comprising engineered repeat sequences. In embodiments, the methods comprise gene-editing proteins comprising one or more CRISPR associated family members. In embodiments, the methods comprise altering the DNA sequence of a cell by transfecting the cell with or inducing the cell to express a gene-editing protein. In embodiments, the methods comprise altering the DNA sequence of a cell that is present in an in vitro culture. In embodiments, the methods comprise altering the DNA sequence of a cell that is present in vivo.

In embodiments, the methods comprise one or more steroids and/or one or more antioxidants in the transfection medium can increase in vivo transfection efficiency, in vivo reprogramming efficiency, and in vivo gene-editing efficiency. In embodiments, the methods comprise contacting a cell or patient with a glucocorticoid, such as hydrocortisone, prednisone, prednisolone, methylprednisolone, dexamethasone or betamethasone. In embodiments, the methods comprise inducing a cell to express a protein of interest by contacting a cell with a medium containing a steroid and contacting the cell with one or more nucleic acid molecules. In embodiments, the nucleic acid molecule comprises synthetic RNA. In embodiments, the steroid is hydrocortisone. In embodiments, the hydrocortisone is present in the medium at a concentration of between about 0.1 uM and about 10 uM, or about 1 uM. In embodiments, the methods comprise inducing a cell in vivo to express a protein of interest by contacting the cell with a medium containing an antioxidant and contacting the cell with one or more nucleic acid molecules. In embodiments, the antioxidant is ascorbic acid or ascorbic-acid-2-phosphate. In embodiments, the ascorbic acid or ascorbic-acid-2-phosphate is present in the medium at a concentration of between about 0.5 mg/L and about 500 mg/L, including about 50 mg/L. In embodiments, the methods comprise reprogramming and/or gene-editing a cell in vivo by contacting the cell with a medium containing a steroid and/or an antioxidant and contacting the cell with one or more nucleic acid molecules, wherein the one or more nucleic acid molecules encodes one or more reprogramming and/or gene-editing proteins. In embodiments, the cell is present in an organism, and the steroid and/or antioxidant are delivered to the organism.

In embodiments, the nucleic acid therapeutic agent encodes a gene-editing protein and/or associated elements for gene-editing functionality. In embodiments, the gene-editing protein is selected from a zinc finger (ZF), transcription activator-like effector (TALE), meganuclease, and clustered regularly interspaced short palindromic repeat (CRISPR)-associated protein. In embodiments, the CRISPR-associated protein is selected from Cas9, CasX, CasY, Cpf1, and gRNA complexes thereof. In embodiments, the CRISPR-associated protein is selected from Cas9, xCas9, Cas12a (Cpf1), Cas13a, Cas14, CasX, CasY, a Class 1 Cas protein, a Class 2 Cas protein, MAD7, and gRNA complexes thereof.

In embodiments, the therapeutic agent is a biologic therapeutic agent. In embodiments, the biologic therapeutic agent is a protein. In embodiments, the biologic therapeutic agent is an interferon, a monoclonal antibody, and/or an interleukin. In embodiments, the biologic therapeutic agent is used to effect immunotherapy selected from one or more of specific active immunotherapy, nonspecific active immunotherapy, passive immunotherapy, and cytotoxic therapy.

In embodiments, the biologic therapeutic agent is a recombinant protein.

In embodiments, the biologic therapeutic agent is a virus.

In embodiments, the biologic therapeutic agent is one of an antibody or an antibody fragment, fusion protein, gene-editing protein, cytokine, antigen, and peptide.

In embodiments, the therapeutic agent is a small molecule therapeutic agent. In embodiments, the small molecule therapeutic agent is one or more of a drug, inhibitor, or cofactor. In embodiments, the drug for use in cancer therapy. In embodiments, the inhibitor is one or more of a kinase inhibitor, proteasome inhibitor, and inhibitor targeting apoptosis.

In embodiments, the therapeutic agent is a vaccine and/or an immunogenic antigen.

### Targeting

Megakaryocyte-derived extracellular vesicles can home to a range of target cells. When megakaryocyte-derived extracellular vesicles bind to a target cell, they can release their cargo via various mechanisms of megakaryocyte-derived extracellular vesicle internalization by the target cell.

In embodiments, the megakaryocyte-derived extracellular vesicles are suitable for homing to bone marrow in vivo. In embodiments, the megakaryocyte-derived extracellular vesicles are suitable for homing to bone marrow in vitro. In embodiments, the megakaryocyte-derived extracellular vesicles home in vivo to bone marrow with about a 2-fold, or about a 3-fold, or about a 4-fold, or about a 5-fold, or about a 6-fold, or about a 7-fold, or about a 8-fold, or about a 9-fold, or about a 10-fold greater specificity than to another cell type, or to another organ, or to all other cell types combined.

In embodiments, the megakaryocyte-derived extracellular vesicles home in vivo to one or more myelopoeitic cells in bone marrow. In embodiments, the one or more myelopoeitic cells are selected from myeloblasts, promyelocytes, neutrophilic myelocytes, eosinophilic myelocytes, neutrophilic metamyelocytes, eosinophilic metamyelocytes, neutrophilic band cells, eosinophilic band cells, segmented neutrophils, segmented eosinophils, segmented basophils, and mast cells. In embodiments, the megakaryocyte-derived extracellular vesicles home in vivo to one or more erythropoietic cells in bone marrow. In embodiments, the one or more erythropoietic cells are selected from pronormoblasts, basophilic normoblasts, polychromatic normoblasts, and orthochromatic normoblasts. In embodiments, the megakaryocyte-derived extracellular vesicles home in vivo to one or more of plasma cells, reticular cells, lymphocytes, monocytes, and megakaryocytes.

In embodiments, the megakaryocyte-derived extracellular vesicles home in vivo to one or more hematopoietic cells in bone marrow. In embodiments, the megakaryocyte-derived extracellular vesicles home in vivo to one or more hematopoietic cells in bone marrow, e.g. thrombopoietic cells.

In embodiments, the megakaryocyte-derived extracellular vesicles home in vivo to one or more hematopoietic stem cells in bone marrow.

In embodiments, the megakaryocyte-derived extracellular vesicles are suitable for homing to an HSC in vivo. In embodiments, the megakaryocyte-derived extracellular vesicles are suitable for homing to an HSC in vitro. In embodiments, the megakaryocyte-derived extracellular vesicles home in vivo to an HSC with about a 2-fold greater specificity than to another cell type, or than to another organ, or than to all other cell types combined. In embodiments, the megakaryocyte-derived extracellular vesicles home in vivo to an HSC with about a 3-fold greater specificity than to another cell type, or than to another organ, or than to all other cell types combined. In embodiments, the megakaryocyte-derived extracellular vesicles home in vivo to an HSC with about a 4-fold greater specificity than to another cell type, or to another organ, or to all other cell types combined. In embodiments, the megakaryocyte-derived extracellular vesicles home in vivo to an HSC with about a 5-fold greater specificity than to another cell type, or to another organ, or to all other cell types combined. In embodiments, the megakaryocyte-derived extracellular vesicles home in vivo to an HSC with about a 6-fold greater specificity than to another cell type, or to another organ, or to all other cell types combined. In embodiments, the megakaryocyte-derived extracellular vesicles home in vivo to an HSC with about a 7-fold greater specificity than to another cell type, or to another organ, or to all other cell types combined. In embodiments, the megakaryocyte-derived extracellular vesicles home in vivo to an HSC with about a 8-fold greater specificity than to another cell type, or to another organ, or to all other cell types combined. In embodiments, the megakaryocyte-derived extracellular vesicles home in vivo to an HSC with about a 9-fold greater specificity than to another cell type, or to another organ, or to all other cell types combined. In embodiments, the megakaryocyte-derived extracellular vesicles home in vivo to an HSC with about a 10-fold greater specificity than to another cell type, or to another organ, or to all other cell types combined.

In embodiments, the megakaryocyte-derived extracellular vesicles are suitable for homing to a lymphatic cell in vivo. In embodiments, the megakaryocyte-derived extracellular vesicles are suitable for homing to a lymphatic cell in vitro. In embodiments, the megakaryocyte-derived extracellular vesicles home in vivo to a lymphatic cell with about a 2-fold greater specificity than to another cell type, or to another organ, or to all other cell types combined. In embodiments, the megakaryocyte-derived extracellular vesicles home in vivo to a lymphatic cell with about a 3-fold greater specificity than to another cell type, or to another organ, or to all other cell types combined. In embodiments, the megakaryocyte-derived extracellular vesicles home in vivo to a lymphatic cell with about a 4-fold greater specificity than to another cell type, or to another organ, or to all other cell types combined. In embodiments, the megakaryocyte-derived extracellular vesicles home in vivo to a lymphatic cell with about a 5-fold greater specificity than to another cell type, or to another organ, or to all other cell types combined. In embodiments, the megakaryocyte-derived extracellular vesicles home in vivo to a lymphatic cell with about a 6-fold greater specificity than to another cell type, or to another organ, or to all other cell types combined. In embodiments, the megakaryocyte-derived extracellular vesicles home in vivo to a lymphatic cell with about a 7-fold greater specificity than to another cell type, or to another organ, or to all other cell types combined. In embodiments, the megakaryocyte-derived extracellular vesicles home in vivo to a lymphatic cell with about a 8-fold greater specificity than to another cell type, or to another organ, or to all other cell types combined. In embodiments, the megakaryocyte-derived extracellular vesicles home in vivo to a lymphatic cell with about a 9-fold greater specificity than to another cell type, or to another organ, or to all other cell types combined. In embodiments, the megakaryocyte-derived extracellular vesicles home in vivo to a lymphatic cell with about a 10-fold greater specificity than to another cell type, or to another organ, or to all other cell types combined.

In embodiments, the megakaryocyte-derived extracellular vesicles are suitable for homing to a regulatory T cell in vivo. In embodiments, the megakaryocyte-derived extracellular vesicles are suitable for homing to a regulatory T cell in vitro. In embodiments, the megakaryocyte-derived extracellular vesicles home in vivo to a regulatory T cell with about a 2-fold greater specificity than to another cell type, or to another organ, or to all other cell types combined. In embodiments, the megakaryocyte-derived extracellular vesicles home in vivo to a regulatory T cell with about a 3-fold greater specificity than to another cell type, or to another organ, or to all other cell types combined. In embodiments, the megakaryocyte-derived extracellular vesicles home in vivo to a regulatory T cell with about a 4-fold greater specificity than to another cell type, or to another organ, or to all other cell types combined. In embodiments, the megakaryocyte-derived extracellular vesicles home in vivo to a regulatory T cell with about a 5-fold greater specificity than to another cell type, or to another organ, or to all other cell types combined. In embodiments, the megakaryocyte-derived extracellular vesicles home in vivo to a regulatory T cell with about a 6-fold greater specificity than to another cell type, or to another organ, or to all other cell types combined. In embodiments, the megakaryocyte-derived extracellular vesicles home in vivo to a regulatory T cell with about a 7-fold greater specificity than to another cell type, or to another organ, or to all other cell types combined. In embodiments, the megakaryocyte-derived extracellular vesicles home in vivo to a regulatory T cell with about a 8-fold greater specificity than to another cell type, or to another organ, or to all other cell types combined. In embodiments, the megakaryocyte-derived extracellular vesicles home in vivo to a regulatory T cell with about a 9-fold greater specificity than to another cell type, or to another organ, or to all other cell types combined. In embodiments, the megakaryocyte-derived extracellular vesicles home in vivo to a regulatory T cell with about a 10-fold greater specificity than to another cell type, or to

In embodiments, the contacting of the deliverable therapeutic agent with the biological cell comprises co-culturing the deliverable therapeutic agent with the biological cell to provide a transfer of the cargo from the deliverable therapeutic agent to the biological cell.

In embodiments, the megakaryocyte-derived extracellular vesicles bind to a cell surface receptor on a cell of the patient. In embodiments, the megakaryocyte-derived extracellular vesicles fuse with the extracellular membrane of a cell of the patient. In embodiments, the megakaryocyte-derived extracellular vesicles are endocytosed by a cell of the patient. In embodiments, the biological cell is one or more of a cancer cell, a tumor cell, a cell infected by a virus, an epithelial cell, an endothelial cell, a nerve cell, a muscle cell, a connective tissue cell, a healthy cell, a diseased cell, a differentiated cell, and a pluripotent cell.

### Definitions

As used herein, “a,” “an,” or “the” can mean one or more than one.

Further, the term “about” when used in connection with a referenced numeric indication means the referenced numeric indication plus or minus up to 10% of that referenced numeric indication. For example, the language “about 50” covers the range of 45 to 55.

An “effective amount,” when used in connection with medical uses is an amount that is effective for providing a measurable treatment, prevention, or reduction in the rate of pathogenesis of a disease of interest.

As used herein, something is “decreased” if a read-out of activity and/or effect is reduced by a significant amount, such as by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100%, in the presence of an agent or stimulus relative to the absence of such modulation. As will be understood by one of ordinary skill in the art, in some embodiments, activity is decreased and some downstream read-outs will decrease but others can increase.

Conversely, activity is “increased” if a read-out of activity and/or effect is increased by a significant amount, for example by at least about 10%, at least about 20%, at least about 30%, at least about 40%, at least about 50%, at least about 60%, at least about 70%, at least about 80%, at least about 90%, at least about 95%, at least about 97%, at least about 98%, or more, up to and including at least about 100% or more, at least about 2-fold, at least about 3-fold, at least about 4-fold, at least about 5-fold, at least about 6-fold, at least about 7-fold, at least about 8-fold, at least about 9-fold, at least about 10-fold, at least about 50-fold, at least about 100-fold, in the presence of an agent or stimulus, relative to the absence of such agent or stimulus.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the compositions and methods of this technology. Similarly, the terms “can” and “may” and their variants are intended to be non-limiting, such that recitation that an embodiment can or may comprise certain elements or features does not exclude other embodiments of the present technology that do not contain those elements or features.

Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

The amount of compositions described herein needed for achieving a therapeutic effect may be determined empirically in accordance with conventional procedures for the particular purpose. Generally, for administering therapeutic agents for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. Therapeutic benefit also includes halting or slowing the progression of the underlying disease or disorder, regardless of whether improvement is realized.

Effective amounts, toxicity, and therapeutic efficacy can be determined by standard pharmaceutical procedures in cell cultures or experimental animals, e.g., for determining the LD50 (the dose lethal to about 50% of the population) and the ED50 (the dose therapeutically effective in about 50% of the population). The dosage can vary depending upon the dosage form employed and the route of administration utilized. The dose ratio between toxic and therapeutic effects is the therapeutic index and can be expressed as the ratio LD50/ED50. In some embodiments, compositions and methods that exhibit large therapeutic indices are preferred. A therapeutically effective dose can be estimated initially from in vitro assays, including, for example, cell culture assays. Also, a dose can be formulated in animal models to achieve a circulating plasma concentration range that includes the IC50 as determined in cell culture, or in an appropriate animal model. Levels of the described compositions in plasma can be measured, for example, by high performance liquid chromatography. The effects of any particular dosage can be monitored by a suitable bioassay. The dosage can be determined by a physician and adjusted, as necessary, to suit observed effects of the treatment.

In certain embodiments, the effect will result in a quantifiable change of at least about 10%, at least about 20%, at least about 30%, at least about 50%, at least about 70%, or at least about 90%. In some embodiments, the effect will result in a quantifiable change of about 10%, about 20%, about 30%, about 50%, about 70%, or even about 90% or more. Therapeutic benefit also includes halting or slowing the progression of the underlying disease or disorder, regardless of whether improvement is realized.

As used herein, “methods of treatment” are equally applicable to use of a composition for treating the diseases or disorders described herein and/or compositions for use and/or uses in the manufacture of a medicaments for treating the diseases or disorders described herein.

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention, and are not to be construed as limiting in any way the remainder of the disclosure.

### Example 1: Yield and Surface Marker Expression of Megakaryocyte-Derived Extracellular Vesicles

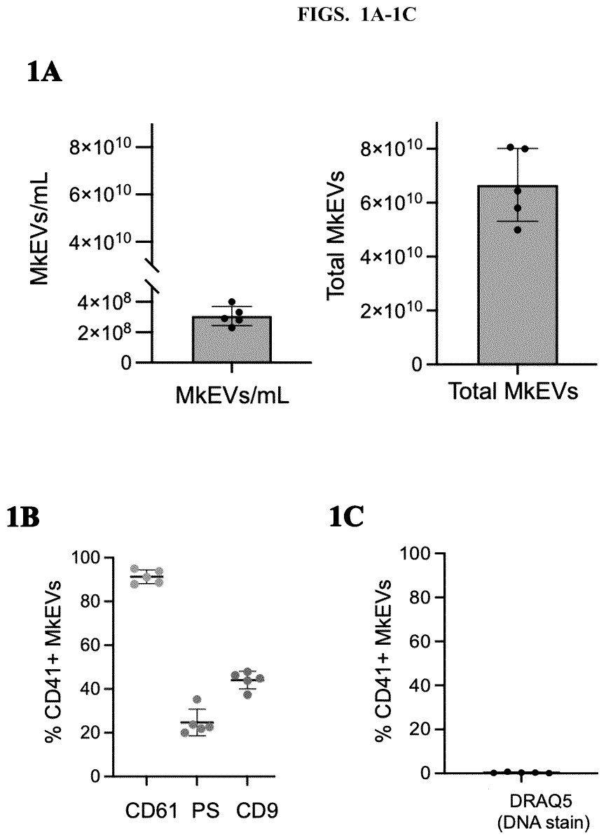

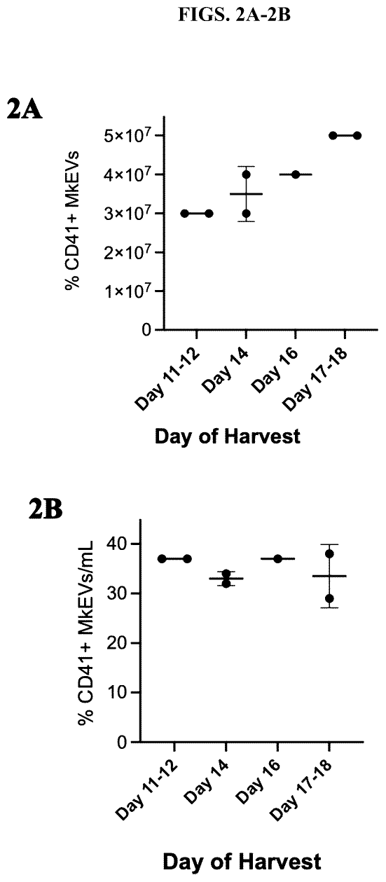

As shown in FIGS. 1A-1C, at harvest days 17/18, the yield and surface marker expression of Megakaryocyte-Derived Extracellular Vesicles (MkEVs) were consistent. The concentration of MkEVs/mL ranged between 2×10⁸ to 4×10⁸ MkEVs/mL and the total MkEV yield were approximately 5×10¹⁰ to 8×10¹⁰ MkEVs (FIG. 1A). In addition, the percent of CD41+ MkEVs expressing individual surface markers were consistent across 5 independent MkEV batches (FIG. 11B). Finally, the MkEV product was DRAQ5 negative by flow cytometry, indicating lack of cellular DNA contamination (FIG. 1C).

As shown in FIGS. 2A-2B, the yield of CD41+ MkEVs continued to rise with increasing days of megakaryocyte differentiation culture. The MkEV yield continued to rise between 11 and 18 days in differentiation culture (FIG. 2A) without a decrease in the purity of CD41+ events (FIG. 2B).

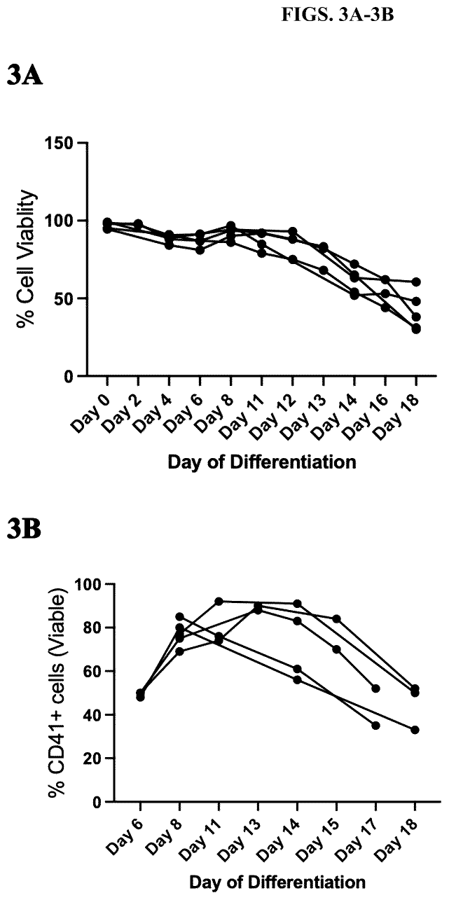

On the other hand, the viability of cells in culture began to decline after differentiation day 12 (FIG. 3A). More specifically, the viability of the CD41+ cell population in culture began to decline between differentiation day 8-13 (FIG. 3B), indicating that the MkEV yield was not linearly correlated with cell viability.

### Example 2: Purity of Megakaryocyte-Derived Extracellular Vesicles

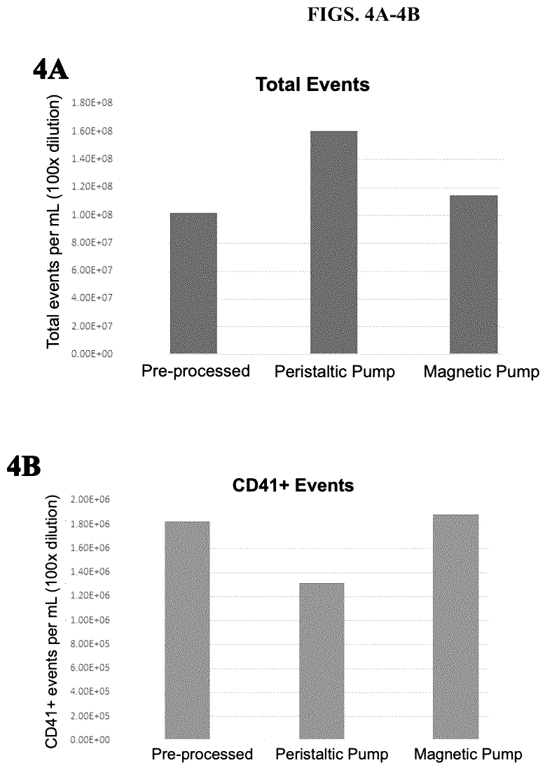

As shown in FIGS. 4A-4B, improved MkEV purity was improved, without sacrificing CD41+ quantity, when a low shear pump was used for tangential flow filtration.

### Example 3: Tuneable Loading of pDNA into MkEVs by Electroporation

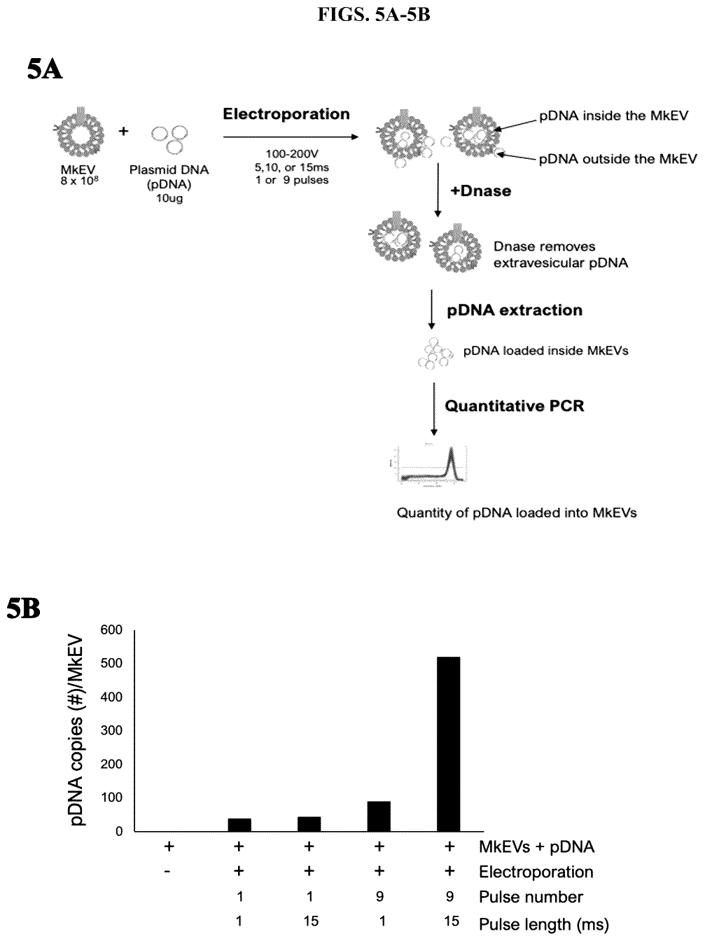

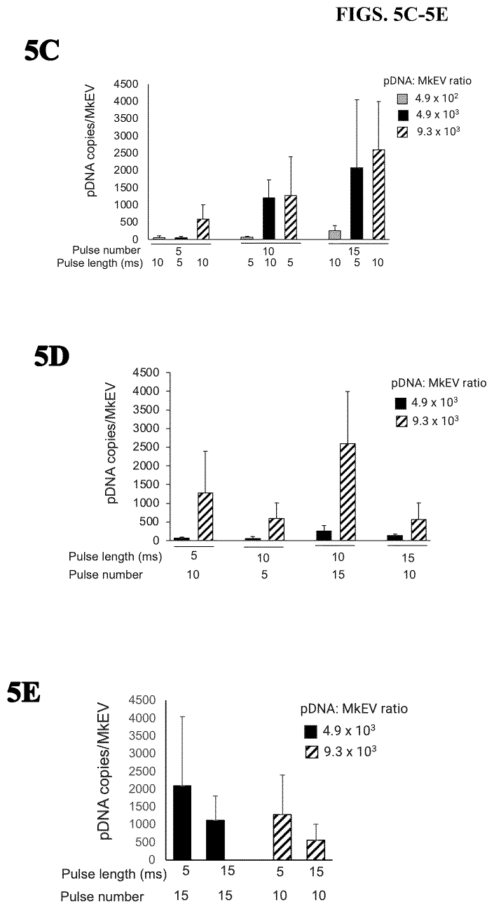

As shown in FIG. 5A, pDNA (5.5 kb) was electroporated into MkEVs. Following electroporation, samples were treated with DNase to remove any free pDNA or MkEV surface-associated pDNA. Following DNAse treatment, DNA was extracted and pDNA amount was quantified by qPCR. The number of pDNA copies were calculated based on a standard curve run in parallel and divided by the total number of MkEVs in the sample to give pDNA copy number/MkEV. MkEVs plus pDNA without electroporation served as a control. Increasing the pulse number (FIGS. 5B and 5C) and increasing the pDNA cargo:MkEV ratio (FIG. 5D) both led to increased pDNA loading. Pulse length of 5 ms provided the highest loading of pDNA into MkEVs while pulse length of 15 ms led to a lower number of pDNA copies loaded into MkEVs (FIG. 5E).

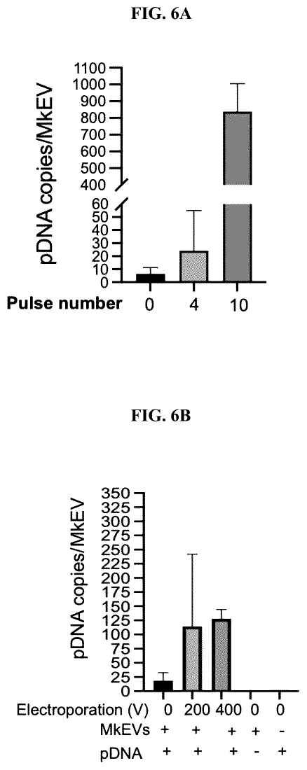

As shown in FIGS. 6A-6B, pDNA between 8-9.8 kb in length were successfully loaded into MkEVs by electroporation. Successful tuneable loading of pDNA (9.8 kb) into MkEVs was achieved with electroporation (FIG. 6A). An average of approximately 24 pDNA copies/MkEV and approximately 840 pDNA copies/MkEV were loaded into MkEVs by electroporation with 4 and 10 pulses, respectively. pDNA (8.3 kb) was successfully electroporated (EP) into MkEVs using either 200V or 400V (FIG. 6B). Controls included MkEVs+ pDNA without electroporation, MkEVs alone, pDNA alone. All samples were treated with DNase to remove any noninternalized pDNA cargo prior to DNA extraction. DNA was then extracted and qPCR was performed. Nanograms of pDNA were calculated based on a standard curve run in parallel and pDNA copy number per MkEV was calculated. Protected pDNA was only recovered from the MkEV+ pDNA+ electroporation sample indicating successful loading with these parameters.

### Example 4: Successful Cargo Loading in to MkEVs

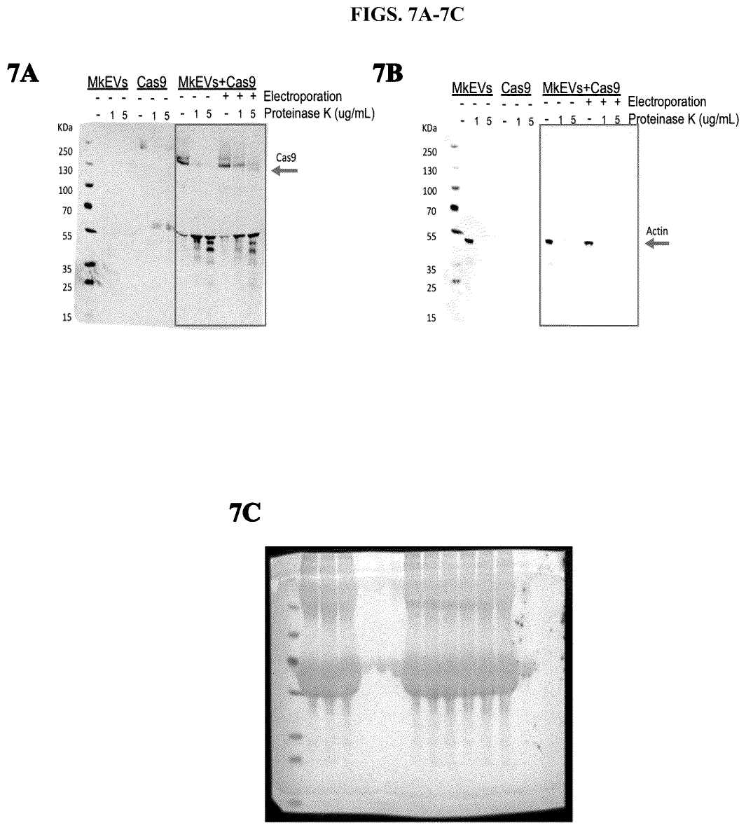

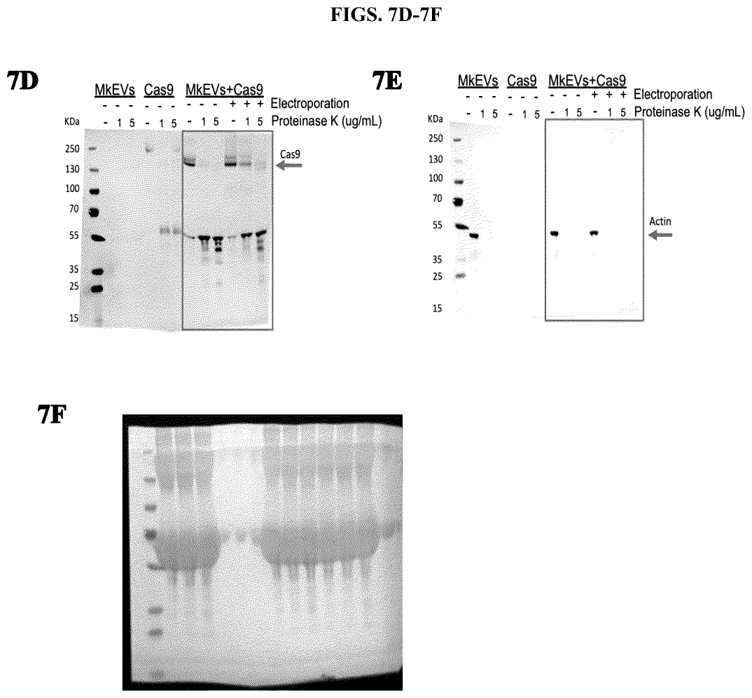

As shown is FIGS. 7A-7F, Cas9 was successfully loaded into MkEVs. MkEVs were loaded with Cas9 by electroporation at 750V (FIGS. 7A-7C) or 1000 V (FIGS. 7D-7F), and subjected to proteinase K treatment at 1 or 5 ug/mL to digest any unbound or externally associated Cas9. Control samples included MkEVs alone±Proteinase K, Cas9 alone±Proteinase K, and MkEVs+cas9 without electroporation±Proteinase K. The MkEVs were then analyzed for internalized, protease-protected Cas9 by western blotting (FIGS. 7A and 7D). Actin was quantified as a non-loaded control (FIGS. 7B and 7E). The absence of Cas9 in un-electroporated but its presence in electroporated samples following protease digestion indicates successful loading of Cas9 inside the MkEV by electroporation. Ponceau staining showed similar loading across all wells (FIGS. 7C and 7F).

### Example 5: Successful Cargo-Loaded MkEVs Cell Targeting

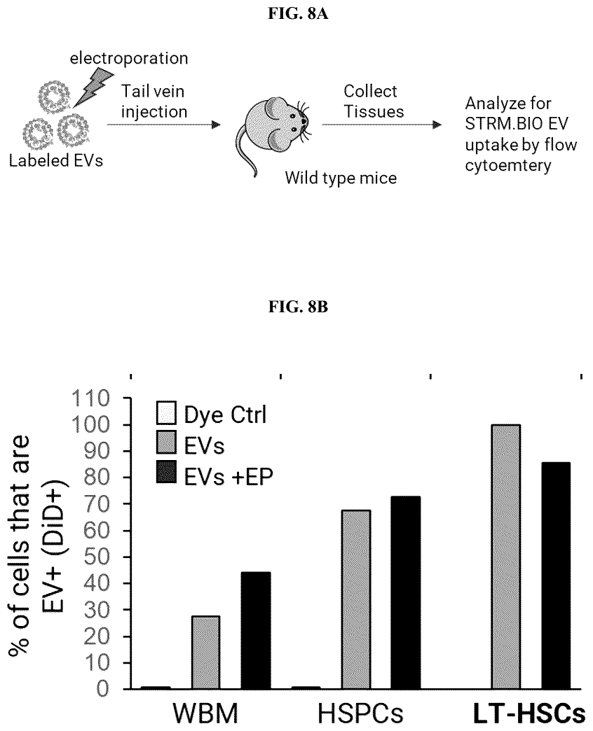

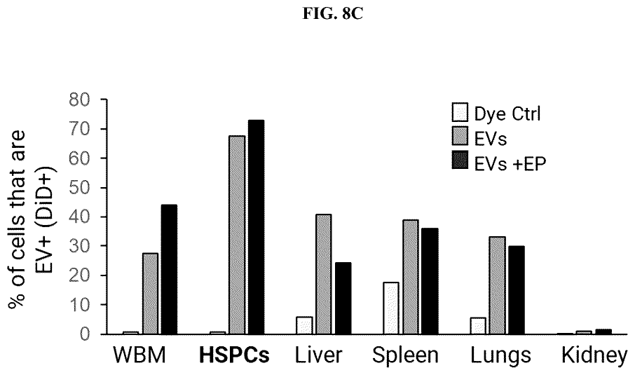

To demonstrate proof of concept that cargo loaded MkEVs (e.g., gene therapy assets) can be delivered to the target cells (e.g., bone marrow cells) following in vivo administration, first, MkEVs were labeled with DiD, subjected to electroporation (1000V, 2 pulses), and then injected intravenously into immunocompetent wild type mice via tail vein injection. DiD-labeled, unelectroporated EVs were injected in parallel to determine the effects of electroporation on biodistribution (FIGS. 8A-8C). Tissues were analyzed 16 hours post injection by flow cytometry to quantify the percent of cells positive for EVs across the different hematopoietic cellular populations within bone marrow (FIG. 8B) and within tissues including liver, spleen, lung, and kidney (FIG. 8C). Injection of DiD processed in parallel without MkEVs served as a negative control. Similar to ex vivo observations, MkEVs preferentially targeted the hematopoietic stem and progenitor cells (HSPCs; Lineage−/c-Kit+/Sca-1+) and long-term hematopoietic stem cells (LT-HSCs; Lineage−/c-Kit+/Sca-1+/CD150+/CD201+). As shown in FIG. 8B, while ˜44% of total whole bone marrow (WBM) was positive for electroporated MKEVs, over 72% and 85% of HSPCs and LTHSCs, respectively, were positive for electroporated MkEVs. Electroporation did not alter the preferential targeting within the targeted tissue (e.g., bone marrow) (FIG. 8B) and did not alter biodistribution in the other tissues analyzed (FIG. 8C).

### EQUIVALENTS

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments described specifically herein. Such equivalents are intended to be encompassed in the scope of the following claims.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

What is claimed is:

1. A method of generating a plurality of megakaryocyte-derived extracellular vesicles, comprising:
a) differentiating human pluripotent stem cells to megakaryocytes; and
b) isolating megakaryocyte-derived extracellular vesicles from the megakaryocytes, wherein:
the isolation is from megakaryocytes in a culture having greater than about 20% viability or more, and/or
the isolation occurs at less than 12 days after commencement of the differentiation or at least about 13 days to about 24 days after commencement of the differentiation.

2. A method of generating a plurality of megakaryocyte-derived extracellular vesicles, comprising:
a) obtaining human pluripotent stem cells, the human pluripotent stem cells being primary CD34+ hematopoietic stem cells;
b) differentiating the human pluripotent stem cells to megakaryocytes; and
c) isolating megakaryocyte-derived extracellular vesicles from the megakaryocytes, wherein:
the isolation is from megakaryocytes in a culture having greater than about 20% viability or more, and/or
the isolation occurs at less than 12 days after commencement of the differentiation or at least about 13 days to about 24 days after commencement of the differentiation.

3. The method according to claim 2, wherein the primary CD34+ hematopoietic stem cells are sourced from peripheral blood or cord blood.

4. The method according to claim 1, wherein the human pluripotent stem cells are selected from induced pluripotent stem cells (iPSCs), hematopoietic stem cells (HSCs), and embryonic stem cells (ESCs).

5. A method of generating a plurality of megakaryocyte-derived extracellular vesicles, comprising:
a) differentiating human pluripotent stem cells to megakaryocytes in culture;
b) enriching the culture for megakaryocytes; and
c) isolating megakaryocyte-derived extracellular vesicles from the megakaryocytes, wherein:
the isolation is from megakaryocytes in a culture having greater than about 20% viability or more, and/or
the isolation occurs at less than 12 days after commencement of the differentiation or at least about 13 days to about 24 days after commencement of the differentiation.

6. The method according to claim 5, wherein the enrichment uses a bead-based selection of megakaryocytes, optionally the beads are coated with an anti-CD61 agent or an anti-CD41 agent.

7. The method according to any one of the preceding claims, wherein the viability is greater than about 25%.

8. The method according to any one of the preceding claims, wherein the wherein the viability is greater than about 30%.

9. The method according to any one of the preceding claims, wherein the viability is greater than about 35%.

10. The method according to any one of the preceding claims, wherein the viability is greater than about 40%.

11. The method according to any one of the preceding claims, wherein the viability is greater than about 45%.

12. The method according to any one of the preceding claims, wherein the viability is greater than about 50%.

13. The method according to any one of the preceding claims, wherein the viability is greater than about 55%.

14. The method according to any one of the preceding claims, wherein the viability is greater than about 60%.

15. The method according to any one of the preceding claims, wherein the viability is greater than about 65%.

16. The method according to any one of the preceding claims, wherein the viability is greater than about 70%.

17. The method according to any one of the preceding claims, wherein the viability is greater than about 75%.

18. The method according to any one of the preceding claims, wherein the viability is greater than about 80%.

19. The method according to any one of the preceding claims, wherein the viability is greater than about 85%.

20. The method according to any one of the preceding claims, wherein the viability is greater than about 90%.

21. The method according to any one of the preceding claims, wherein the viability is greater than about 95%.

22. The method according to any one of claims 1-6, wherein the viability is about 50% or less, e.g. about 50%, or about 45%, or about 40%, or about 30%.

23. The method according to any one of the preceding claims, wherein the isolation occurs at about 7 days after commencement of the differentiation.

24. The method according to any one of the preceding claims, wherein the isolation occurs at about 8 days after commencement of the differentiation.

25. The method according to any one of the preceding claims, wherein the isolation occurs at about 9 days after commencement of the differentiation.

26. The method according to any one of the preceding claims, wherein the isolation occurs at about 10 days after commencement of the differentiation.

27. The method according to any one of the preceding claims, wherein the isolation occurs at about 14 days after commencement of the differentiation or at least about 14 days after commencement of the differentiation.

28. The method according to any one of the preceding claims, wherein the isolation occurs at about 15 days after commencement of the differentiation or at least about 15 days after commencement of the differentiation.

29. The method according to any one of the preceding claims, wherein the isolation occurs at about 16 days after commencement of the differentiation or at least about 16 days after commencement of the differentiation.

30. The method according to any one of the preceding claims, wherein the isolation occurs at about 17 days after commencement of the differentiation or at least about 17 days after commencement of the differentiation.

31. The method according to any one of the preceding claims, wherein the isolation occurs at about 18 days after commencement of the differentiation or at least about 18 days commencement of the differentiation.

32. The method according to any one of the preceding claims, wherein the isolation occurs at about 19 days after commencement of the differentiation or at least about 19 days after commencement of the differentiation.

33. The method according to any one of the preceding claims, wherein the isolation occurs at about 20 days after commencement of the differentiation or at least about 20 days after commencement of the differentiation.

34. The method according to any one of the preceding claims, wherein the isolation occurs at about 21 days after commencement of the differentiation or at least about 21 days after commencement of the differentiation.

35. The method according to any one of the preceding claims, wherein the isolation occurs at about 22 days after commencement of the differentiation or at least about 22 days after commencement of the differentiation.

36. The method according to any one of the preceding claims, wherein the isolation occurs at about 23 days after commencement of the differentiation or at least about 23 days after commencement of the differentiation.

37. The method according to any one of the preceding claims, wherein the isolation occurs at about 24 days after commencement of the differentiation or at least about 24 days after commencement of the differentiation.

38. The method according to any one of the preceding claims, wherein the method yields more megakaryocyte-derived extracellular vesicles than a comparable method with megakaryocytes in a culture having greater than 40% viability.

39. The method according to any one of the preceding claims, wherein the method yields more megakaryocyte-derived extracellular vesicles than a comparable method with megakaryocytes in a culture having greater than 50% viability.

40. The method according to any one of the preceding claims, wherein the method yields more megakaryocyte-derived extracellular vesicles than a comparable method with megakaryocytes in a culture having greater than 60% viability.

41. The method according to any one of the preceding claims, wherein the method yields more megakaryocyte-derived extracellular vesicles than a comparable method with megakaryocytes in a culture having greater than 70% viability.

42. The method according to any one of the preceding claims, wherein the method yields more megakaryocyte-derived extracellular vesicles than a comparable method with megakaryocytes in a culture having greater than 80% viability.

43. The method according to any one of the preceding claims, wherein the method yields more megakaryocyte-derived extracellular vesicles than a comparable method with megakaryocytes in a culture having greater than 90% viability.

44. The method according to any one of the preceding claims, wherein the method yields more megakaryocyte-derived extracellular vesicles than a comparable method in which isolation occurs at least less than 20 days after commencement of the differentiation.

45. The method according to any one of the preceding claims, wherein the method yields more megakaryocyte-derived extracellular vesicles than a comparable method in which isolation occurs at least less than 19 days after commencement of the differentiation.

46. The method according to any one of the preceding claims, wherein the method yields more megakaryocyte-derived extracellular vesicles than a comparable method in which isolation occurs at least less than 18 days after commencement of the differentiation.

47. The method according to any one of the preceding claims, wherein the method yields more megakaryocyte-derived extracellular vesicles than a comparable method in which isolation occurs at least less than 17 days after commencement of the differentiation.

48. The method according to any one of the preceding claims, wherein the method yields more megakaryocyte-derived extracellular vesicles than a comparable method in which isolation occurs at least less than 16 days after commencement of the differentiation.

49. The method according to any one of the preceding claims, wherein the method yields more megakaryocyte-derived extracellular vesicles than a comparable method in which isolation occurs at least less than 15 days after commencement of the differentiation.

50. The method according to any one of the preceding claims, wherein the method yields more megakaryocyte-derived extracellular vesicles than a comparable method in which isolation occurs at day 14 after commencement of the differentiation.

51. The method according to any one of the preceding claims, wherein the method yields more megakaryocyte-derived extracellular vesicles than a comparable method in which isolation occurs at day 13 after commencement of the differentiation.

52. The method according to any one of the preceding claims, wherein the method yields more megakaryocyte-derived extracellular vesicles than a comparable method in which isolation occurs at day 12 after commencement of the differentiation.

53. The method according to any one of the preceding claims, wherein the method yields more megakaryocyte-derived extracellular vesicles than a comparable method in which isolation occurs at day 11 after commencement of the differentiation.

54. The method according to any one of the preceding claims, wherein the megakaryocyte-derived extracellular vesicles comprise a lipid bilayer membrane surrounding a lumen, wherein:
a) the lumen comprises one or more megakaryocyte-derived nucleic acid molecules selected from mRNA, tRNA, rRNA, siRNA, microRNA, regulating RNA, and non-coding and coding RNA and
b) the lipid bilayer membrane comprises one or more proteins associated with or embedded within.

55. The method according to claim 54, wherein the lipid bilayer membrane comprises one or more proteins selected from CD54, CD18, CD43, CD11b, CD62P, CD41, CD61, CD21, CD51, CLEC-2, LAMP-1 (CD107a), CD63, CD42b, CD9, CD31, CD47, CD147, CD32a, and GPVI and/or the lipid bilayer membrane comprises phosphatidylserine.

56. The method according to claim 55, wherein:
a) greater than about 30%, or about 40%, or about 50%, or about 60%, or about 70%, or about 80%, or about 90%, or about 95% of the megakaryocyte-derived extracellular vesicles comprise a lipid bilayer membrane comprising CD41 and/or
b) greater than about 50%, or about 60%, or about 70%, or about 80%, or about 90%, or about 95% of the megakaryocyte-derived extracellular vesicles comprise a lipid bilayer membrane comprising CD61.

57. The method according to any one of the preceding claims, wherein the megakaryocyte-derived extracellular vesicles are substantially of a diameter in the range between about 100 nm to about 600 nm.

58. The method according to any one of the preceding claims, wherein the megakaryocyte-derived extracellular vesicles are substantially of a diameter in the range between about 30 nm to about 100 nm.

59. The method according to any one of the preceding claims, wherein the megakaryocyte-derived extracellular vesicles are substantially of a diameter in the range between about 100 nm to about 300 nm.

60. The method according to any one of the preceding claims, wherein about 90% or more, or about 95% or more, or about 97% or more, or about 99% or more of the megakaryocyte-derived extracellular vesicles are of a diameter of between about 100 nm and about 600 nm.

61. The method according to any one of the preceding claims, wherein about 90% or more, or about 95% or more, or about 97% or more, or about 99% or more of the megakaryocyte-derived extracellular vesicles are of a diameter of between about 100 nm and about 300 nm.

62. The method according to any one of the preceding claims, wherein the megakaryocyte-derived extracellular vesicles are substantially free of autologous DNA.

63. The method according to any one of the preceding claims, wherein the megakaryocyte-derived extracellular vesicles are substantially free of:
a) megakaryocytes, and/or
b) platelets.

64. The method according to any one of the preceding claims, wherein the megakaryocyte-derived extracellular vesicles are suitable for homing to a hematopoietic stem cell in vivo and/or in vitro.

65. The method according to any one of the preceding claims, wherein the megakaryocyte-derived extracellular vesicles are suitable for homing to bone marrow in vivo and/or in vitro.

66. The method according to any one of the preceding claims, wherein the megakaryocyte-derived extracellular vesicles are suitable for homing to a lymphatic cell in vivo and/or in vitro.

67. The method according to any one of the preceding claims, wherein the megakaryocyte-derived extracellular vesicles are suitable for homing to a regulatory T cell in vivo and/or in vitro.

68. The method according to any one of the preceding claims, wherein the megakaryocyte-derived extracellular vesicles are suitable for loading with cargo into the lumen and/or loading with cargo associated with the surface of the megakaryocyte-derived extracellular vesicles.

69. The method according to claim 68, wherein the cargo is one or more therapeutic agents.

70. The method according to claim 69, wherein the therapeutic agent is a nucleic acid therapeutic agent.

71. The method according to claim 70, wherein the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids.

72. The method according to any one of claims 70-71, wherein the nucleic acid therapeutic agent is mRNA, and optionally: is in vitro transcribed or synthetic and/or comprises one or more non-canonical nucleotides, optionally selected from pseudouridine and 5-methoxyuridine.

73. The method according to any one of claims 70-72, wherein the nucleic acid therapeutic agent encodes a functional protein.

74. The method according to any one of claims 70-73, wherein the nucleic acid therapeutic agent encodes a gene-editing protein and/or associated elements for gene-editing functionality.

75. The method according to claim 74, wherein the gene-editing protein is selected from a zinc finger (ZF), transcription activator-like effector (TALE), meganuclease, and clustered regularly interspaced short palindromic repeat (CRISPR)-associated protein.

76. The method according to claim 75, wherein the CRISPR-associated protein is selected from Cas9, CasX, CasY, Cpf1, and gRNA complexes thereof.

77. The method according to claim 69, wherein the therapeutic agent is a biologic therapeutic agent.

78. The method according to claim 77, wherein the biologic therapeutic agent is a protein.

79. The method according to any one of claims 77-78, wherein the biologic therapeutic agent is a recombinant protein.

80. The method according to any one of claims 77-79, wherein the biologic therapeutic agent is one of an antibody or an antibody fragment, fusion protein, gene-editing protein, cytokine, antigen, and peptide.

81. The method according to claim 69, wherein the therapeutic agent is a small molecule therapeutic agent.

82. The method according to claim 69, wherein the therapeutic agent is a vaccine and/or an immunogenic antigen.

83. A method for purifying a plurality of megakaryocyte-derived extracellular vesicles, comprising:
a) obtaining a material comprising a population of megakaryocytes in culture or the cell culture media thereof,
b) filtering the material of step (a) thereby yielding predominantly CD41+ megakaryocyte-derived extracellular vesicles in the filtrate.

84. The method according to claim 83, wherein the filtering is achieved by using tangential flow filtration.

85. The method according to claim 84, wherein the tangential flow filtration is peristaltic.

86. The method according to claim 84, wherein the tangential flow filtration is substantially low-shear.

87. The method according to claim 84, wherein the tangential flow filtration is substantially low-shear and substantially pulsation-free.

88. The method according to any one of claims 84, or 86-87, wherein the tangential flow filtration is non-peristaltic.

89. The method according to any one of claims 83-88, wherein the method is substantially devoid of compression on the vesicles.

90. The method according to any one of claims 83-89, wherein the method is substantially devoid of compression on the biogenesis of vesicles.

91. The method according to any one of claims 83-90, wherein the filtering is achieved by using a low-pass acoustic filter.

92. The method according to any one of claims 83-90, wherein the filtering is achieved by using a cross-flow membrane filtration.

93. The method according to any one of claims 83-90, wherein the filtering is achieved by using a counterflow centrifugation elutriation.

94. The method according to any one of claims 83-90, wherein the filtering is achieved by using a centrifugal pump that is based on magnetic levitation principles.

95. The method according to any one of claims 83-94, wherein the method yields a preparation of vesicles that is greater than about 40% CD41+.

96. The method according to any one of claims 83-95, wherein the method yields a preparation of vesicles that is devoid of CD41− vesicles.

97. The method according to any one of claims 83-96, wherein the method yields a plurality of megakaryocyte-derived extracellular vesicles that is substantially devoid of non-megakaryocyte-derived extracellular vesicles.

98. The method according to any one of claims 83-97, wherein the method yields a plurality of megakaryocyte-derived extracellular vesicles that is substantially intact.

99. The method according to any one of claims 83-98, wherein the method yields a plurality of megakaryocyte-derived extracellular vesicles that is suitable for cargo loading.

100. The method according to any one of claims 83-99, the megakaryocyte-derived extracellular vesicles comprise a lipid bilayer membrane surrounding a lumen, wherein:
a) the lumen comprises one or more megakaryocyte-derived nucleic acid molecules selected from mRNA, tRNA, rRNA, siRNA, microRNA, regulating RNA, and non-coding and coding RNA and
b) the lipid bilayer membrane comprises one or more proteins associated with or embedded within.

101. The method according to claim 100, wherein the lipid bilayer membrane comprises one or more proteins selected from CD54, CD18, CD43, CD11b, CD62P, CD41, CD61, CD21, CD51, CLEC-2, LAMP-1 (CD107a), CD63, CD42b, CD9, CD31, CD47, CD147, CD32a, and GPVI and/or the lipid bilayer membrane comprises phosphatidylserine.

102. The method according to claim 101, wherein greater than about 30%, or about 40%, or about 50%, or about 60%, or about 70%, or about 80%, or about 90%, or about 95% of the megakaryocyte-derived extracellular vesicles comprise a lipid bilayer membrane comprising CD41.

103. The method according to any one of claims 83-102, wherein the megakaryocyte-derived extracellular vesicles are substantially of a diameter in the range between about 100 nm to about 600 nm.

104. The method according to any one of claims 83-103, wherein the megakaryocyte-derived extracellular vesicles are substantially of a diameter in the range between about 30 nm to about 100 nm.

105. The method according to any one of claims 83-104, wherein the megakaryocyte-derived extracellular vesicles are substantially of a diameter in the range between about 100 nm to about 300 nm.

106. The method according to any one of claims 83-105, wherein about 90% or more, or about 95% or more, or about 97% or more, or about 99% or more of the megakaryocyte-derived extracellular vesicles are of a diameter of between about 100 nm and about 600 nm.

107. The method according to any one of claims 83-106, wherein about 90% or more, or about 95% or more, or about 97% or more, or about 99% or more of the megakaryocyte-derived extracellular vesicles are of a diameter of between about 100 nm and about 300 nm.

108. The method according to any one of claims 83-107, wherein the megakaryocyte-derived extracellular vesicles are substantially free of autologous DNA.

109. The method according to any one of claims 83-108, wherein the megakaryocyte-derived extracellular vesicles are substantially free of:
a) megakaryocytes, and/or
b) platelets.

110. The method according to any one of claims 83-109, wherein the megakaryocyte-derived extracellular vesicles are suitable for homing to a hematopoietic stem cell in vivo and/or in vitro.

111. The method according to any one of claims 83-109, wherein the megakaryocyte-derived extracellular vesicles are suitable for homing to bone marrow in vivo and/or in vitro.

112. The method according to any one of claims 83-111, wherein the megakaryocyte-derived extracellular vesicles are suitable for homing to a lymphatic cell in vivo and/or in vitro.

113. The method according to any one of claims 83-112, wherein the megakaryocyte-derived extracellular vesicles are suitable for homing to a regulatory T cell in vivo and/or in vitro.

114. The method according to any one of claims 83-113, wherein the megakaryocyte-derived extracellular vesicles are suitable for loading with cargo into the lumen and/or loading with cargo associated with the surface of the megakaryocyte-derived extracellular vesicles.

115. The method according to claim 114, wherein the cargo is one or more therapeutic agents.

116. The method according to according to claim 115, wherein the therapeutic agent is a nucleic acid therapeutic agent.

117. The method according to claim 116, wherein the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids.

118. The method according to any one of claims 116-117, wherein the nucleic acid therapeutic agent is mRNA, and optionally: is in vitro transcribed or synthetic and/or comprises one or more non-canonical nucleotides, optionally selected from pseudouridine and 5-methoxyuridine.

119. The method according to any one of claims 116-118, wherein the nucleic acid therapeutic agent encodes a functional protein.

120. The method according to any one of claims 116-119, wherein the nucleic acid therapeutic agent encodes a gene-editing protein and/or associated elements for gene-editing functionality.

121. The method according to claim 119, wherein the gene-editing protein is selected from a zinc finger (ZF), transcription activator-like effector (TALE), meganuclease, and clustered regularly interspaced short palindromic repeat (CRISPR)-associated protein.

122. The method according to claim 121, wherein the CRISPR-associated protein is selected from Cas9, CasX, CasY, Cpf1, and gRNA complexes thereof.

123. The method according to claim 115, wherein the therapeutic agent is a biologic therapeutic agent.

124. The method according to claim 123, wherein the biologic therapeutic agent is a protein.

125. The method according to any one of claims 123-124, wherein the biologic therapeutic agent is a recombinant protein.

126. The method according to any one of claims 123-125, wherein the biologic therapeutic agent is one of an antibody or an antibody fragment, fusion protein, gene-editing protein, cytokine, antigen, and peptide.

127. The method according to claim 115, wherein the therapeutic agent is a small molecule therapeutic agent.

128. The method according to claim 115, wherein the therapeutic agent is a vaccine and/or an immunogenic antigen.

129. A method of loading cargo in a plurality of megakaryocyte-derived extracellular vesicles, comprising:
(a) obtaining a plurality of megakaryocyte-derived extracellular vesicles;
(b) contacting the plurality of megakaryocyte-derived extracellular vesicles with a cargo of interest; and
(c) applying an electrical pulse for a period of time, thereby permitting the cargo to pass into the lumen of the megakaryocyte-derived extracellular vesicles, wherein:
the electrical pulse is applied for about 5 to about 25 milliseconds and
the electrical pulse is applied about 5 to about 25 times.

130. The method of claim 129, wherein:
the electrical pulse is applied for about 10 to about 20 milliseconds and
the electrical pulse is applied about 10 to about 20 times.

131. A method of loading cargo in a plurality of megakaryocyte-derived extracellular vesicles, comprising:
(a) obtaining a plurality of megakaryocyte-derived extracellular vesicles;
(b) contacting the plurality of megakaryocyte-derived extracellular vesicles with a cargo of interest; and
(c) applying an electrical pulse for a period of time, thereby permitting the cargo to pass into the lumen of the megakaryocyte-derived extracellular vesicles, wherein:
the electrical pulse is applied for at least about 15 milliseconds and
the electrical pulse is applied for at least about 9 times.

132. The method of claim 131, wherein the pulse is applied for about 15 milliseconds.

133. The method of claim 131 or 132, wherein the pulse is applied about 10 times.

134. The method according to any one of claims 129-133, the megakaryocyte-derived extracellular vesicles comprise a lipid bilayer membrane surrounding a lumen, wherein:
a) the lumen comprises one or more megakaryocyte-derived nucleic acid molecules selected from mRNA, tRNA, rRNA, siRNA, microRNA, regulating RNA, and non-coding and coding RNA and
b) the lipid bilayer membrane comprises one or more proteins associated with or embedded within.

135. The method according to claim 134, wherein the lipid bilayer membrane comprises one or more proteins selected from CD54, CD18, CD43, CD11b, CD62P, CD41, CD61, CD21, CD51, CLEC-2, LAMP-1 (CD107a), CD63, CD42b, CD9, CD31, CD47, CD147, CD32a, and GPVI and/or the lipid bilayer membrane comprises phosphatidylserine.

136. The method according to claim 135, wherein greater than about 50%, or about 60%, or about 70%, or about 80%, or about 90%, or about 95% of the megakaryocyte-derived extracellular vesicles comprise a lipid bilayer membrane comprising CD41.

137. The method according to any one of claims 129-136, wherein the megakaryocyte-derived extracellular vesicles are substantially of a diameter in the range between about 100 nm to about 600 nm.

138. The method according to any one of claims 129-137, wherein the megakaryocyte-derived extracellular vesicles are substantially of a diameter in the range between about 30 nm to about 100 nm.

139. The method according to any one of claims 129-138, wherein the megakaryocyte-derived extracellular vesicles are substantially of a diameter in the range between about 100 nm to about 300 nm.

140. The method according to any one of claims 129-139, wherein about 90% or more, or about 95% or more, or about 97% or more, or about 99% or more of the megakaryocyte-derived extracellular vesicles are of a diameter of between about 100 nm and about 600 nm.

141. The method according to any one of claims 129-140, wherein about 90% or more, or about 95% or more, or about 97% or more, or about 99% or more of the megakaryocyte-derived extracellular vesicles are of a diameter of between about 100 nm and about 300 nm.

142. The method according to any one of claims 129-141, wherein the megakaryocyte-derived extracellular vesicles are substantially free of autologous DNA.

143. The method according to any one of claims 129-142, wherein the megakaryocyte-derived extracellular vesicles are substantially free of:
a) megakaryocytes, and/or
b) platelets.

144. The method according to any one of claims 129-143, wherein the megakaryocyte-derived extracellular vesicles are suitable for homing to a hematopoietic stem cell in vivo and/or in vitro.

145. The method according to any one of claims 129-144, wherein the megakaryocyte-derived extracellular vesicles are suitable for homing to bone marrow in vivo and/or in vitro.

146. The method according to any one of claims 129-145, wherein the megakaryocyte-derived extracellular vesicles are suitable for homing to a lymphatic cell in vivo and/or in vitro.

147. The method according to any one of claims 129-146, wherein the megakaryocyte-derived extracellular vesicles are suitable for homing to a regulatory T cell in vivo and/or in vitro.

148. The method according to claim 129-147, wherein the cargo is one or more therapeutic agents.

149. The method according to according to claim 148, wherein the therapeutic agent is a nucleic acid therapeutic agent.

150. The method according to claim 149, wherein the nucleic acid therapeutic agent is selected from one or more non-autologous and/or recombinant nucleic acid constructs selected from mRNA, tRNA, rRNA, siRNA, microRNA, regulating RNA, non-coding and coding RNA, linear DNA, DNA fragments, or DNA plasmids.

151. The method according to any one of claims 149-150, wherein the nucleic acid therapeutic agent is mRNA, and optionally: is in vitro transcribed or synthetic and/or comprises one or more non-canonical nucleotides, optionally selected from pseudouridine and 5-methoxyuridine.

152. The method according to any one of claims 149-151, wherein the nucleic acid therapeutic agent encodes a functional protein.

153. The method according to any one of claims 149-152, wherein the nucleic acid therapeutic agent encodes a gene-editing protein and/or associated elements for gene-editing functionality.

154. The method according to claim 153, wherein the gene-editing protein is selected from a zinc finger (ZF), transcription activator-like effector (TALE), meganuclease, and clustered regularly interspaced short palindromic repeat (CRISPR)-associated protein.

155. The method according to claim 154, wherein the CRISPR-associated protein is selected from Cas9, CasX, CasY, Cpf1, and gRNA complexes thereof.

156. The method according to claim 148, wherein the therapeutic agent is a biologic therapeutic agent.

157. The method according to claim 156, wherein the biologic therapeutic agent is a protein.

158. The method according to any one of claims 156-157, wherein the biologic therapeutic agent is a recombinant protein.

159. The method according to any one of claims 156-158, wherein the biologic therapeutic agent is one of an antibody or an antibody fragment, fusion protein, gene-editing protein, cytokine, antigen, and peptide.

160. The method according to claim 148, wherein the therapeutic agent is a small molecule therapeutic agent.

161. The method according to claim 148, wherein the therapeutic agent is a vaccine and/or an immunogenic antigen.

162. A method of generating a plurality of megakaryocyte-derived extracellular vesicles, comprising:
a) differentiating human pluripotent stem cells to megakaryocytes; and
b) isolating megakaryocyte-derived extracellular vesicles from the megakaryocytes, wherein:
the isolation is from megakaryocytes in a culture having greater than about 20% viability or more, and
the isolation occurs at 17 or 18 days after commencement of the differentiation.

163. A method of generating a plurality of megakaryocyte-derived extracellular vesicles, comprising:
a) obtaining human pluripotent stem cells, the human pluripotent stem cells being primary CD34+ hematopoietic stem cells;
b) differentiating the human pluripotent stem cells to megakaryocytes; and
c) isolating megakaryocyte-derived extracellular vesicles from the megakaryocytes, wherein:
the isolation is from megakaryocytes in a culture having greater than about 20% viability or more, and
the isolation occurs at 17 or 18 days after commencement of the differentiation.

164. A method of generating a plurality of megakaryocyte-derived extracellular vesicles, comprising:
a) differentiating human pluripotent stem cells to megakaryocytes in culture;
b) enriching the culture for megakaryocytes; and
c) isolating megakaryocyte-derived extracellular vesicles from the megakaryocytes, wherein:
the isolation is from megakaryocytes in a culture having greater than about 20% viability or more, and
the isolation occurs isolation occurs at 17 or 18 days after commencement of the differentiation.

165. A method of loading cargo in a plurality of megakaryocyte-derived extracellular vesicles, comprising:
a) obtaining a plurality of megakaryocyte-derived extracellular vesicles;
b) contacting the plurality of megakaryocyte-derived extracellular vesicles with a cargo of interest; and
c) applying an electrical pulse for a period of time, thereby permitting the cargo to pass into the lumen of the megakaryocyte-derived extracellular vesicles, wherein:
the electrical pulse is applied for about 5 milliseconds and
the electrical pulse is applied about 4 to about 10 times.
